SEQUENCE LISTING (94 sequences)

SEQ ID NO: 1 - skipped

SEQ ID NO: 2 - protein, 14 aa, synthetic construct
  REGION 1..14: note = Synthetically generated peptide
  MOD_RES 1: note = allylglycine
  MOD_RES 6: note = allylglycine
  MOD_RES 14: note = AMIDATION
GCELCGNPACTGCY

SEQ ID NO: 3 - protein, 20 aa, synthetic construct
  REGION 1..20: note = Synthetically generated peptide
  MOD_RES 1: note = N-terminus capped with C12 alkyl carboxylic acid
  MOD_RES 1: note = glutamic acid wherein the side chain carboxylic acid forms the peptide linkage
  MOD_RES 2: note = lysine wherein the side chain amine forms the peptide linkage
  MOD_RES 20: note = AMIDATION
EKNSSYCCELCCNPACTGCY

SEQ ID NO: 4 - protein, 20 aa, synthetic construct
  REGION 1..20: note = Synthetically generated peptide
  MOD_RES 1: note = N-terminus capped with C16 alkyl carboxylic acid
  MOD_RES 1: note = glutamic acid wherein the side chain carboxylic acid forms the peptide linkage
  MOD_RES 2: note = lysine wherein the side chain amine forms the peptide linkage
  MOD_RES 20: note = AMIDATION
EKNSSYCCELCCNPACTGCY

SEQ ID NO: 5 - protein, 20 aa, synthetic construct
  REGION 1..20: note = Synthetically generated peptide
  MOD_RES 1: note = N-terminus capped with C12 alkyl carboxylic acid
  MOD_RES 1: note = glutamic acid wherein the side chain carboxylic acid forms the peptide linkage
  MOD_RES 2: note = lysine wherein the side chain amine forms the peptide linkage
EKNSSYCCELCCNPACTGCY

SEQ ID NO: 6 - protein, 20 aa, synthetic construct
  REGION 1..20: note = Synthetically generated peptide
  MOD_RES 1: note = N-terminus capped with C14 alkyl carboxylic acid
  MOD_RES 1: note = glutamic acid wherein the side chain carboxylic acid forms the peptide linkage
  MOD_RES 2: note = lysine wherein the side chain amine forms the peptide linkage
EKNSSYCCELCCNPACTGCY

SEQ ID NO: 7 - protein, 20 aa, synthetic construct
  REGION 1..20: note = Synthetically generated peptide
  MOD_RES 1: note = N-terminus capped with C16 alkyl carboxylic acid
  MOD_RES 1: note = glutamic acid wherein the side chain carboxylic acid forms the peptide linkage
  MOD_RES 2: note = lysine wherein the side chain amine forms the peptide linkage
EKNSSYCCELCCNPACTGCY

SEQ ID NO: 8 - protein, 14 aa, synthetic construct
  REGION 1..14: note = Synthetically generated peptide
  MOD_RES 2: note = allylglycine
  MOD_RES 10: note = allylglycine
  MOD_RES 14: note = AMIDATION
CGELCCNPAGTGCY

SEQ ID NO: 9 - protein, 16 aa, synthetic construct
  REGION 1..16: note = Synthetically generated peptide
  MOD_RES 4: note = allylglycine
  MOD_RES 12: note = allylglycine
  MOD_RES 16: note = AMIDATION
NDDGELCVNVAGTGCL

SEQ ID NO: 10 - protein, 16 aa, synthetic construct
  REGION 1..16: note = Synthetically generated peptide
  MOD_RES 7: note = allylglycine
  MOD_RES 15: note = allylglycine
  MOD_RES 16: note = AMIDATION
NDDCELGVNVACTGGL

SEQ ID NO: 11 - protein, 20 aa, synthetic construct
  REGION 1..20: note = Synthetically generated peptide
  MOD_RES 1: note = N-terminus capped with C18 alkyl carboxylic acid
  MOD_RES 1: note = glutamic acid wherein the side chain carboxylic acid forms the peptide linkage
  MOD_RES 2: note = lysine wherein the side chain amine forms the peptide linkage
EKNSSYCCELCCNPACTGCY

SEQ ID NO: 12 - protein, 13 aa, synthetic construct
  REGION 1..13: note = Synthetically generated peptide
  MOD_RES 1: note = allylglycine
  MOD_RES 6: note = allylglycine
  MOD_RES 13: note = AMIDATION
GCELCGNPACTGC

SEQ ID NO: 13 - protein, 13 aa, synthetic construct
  REGION 1..13: note = Synthetically generated peptide
  MOD_RES 1: note = allylglycine
  MOD_RES 6: note = allylglycine
  MOD_RES 13: note = AMIDATION
GCELCGNPACAGC

SEQ ID NO: 14 - protein, 14 aa, synthetic construct
  REGION 1..14: note = Synthetically generated peptide
  MOD_RES 1: note = allylglycine
  MOD_RES 6: note = allylglycine
  MOD_RES 14: note = AMIDATION
GCEFCGNPACTGCY

SEQ ID NO: 15 - protein, 13 aa, synthetic construct
  REGION 1..13: note = Synthetically generated peptide
  MOD_RES 1: note = allylglycine
  MOD_RES 4: note = 4-fluorophenylalanine
  MOD_RES 6: note = allylglycine
  MOD_RES 13: note = AMIDATION
GCEFCGNPACTGC

SEQ ID NO: 16 - protein, 14 aa, synthetic construct
  REGION 1..14: note = Synthetically generated peptide
  MOD_RES 1: note = allylglycine
  MOD_RES 4: note = 4-fluorophenylalanine
  MOD_RES 6: note = allylglycine
  MOD_RES 14: note = AMIDATION
GCEFCGNPACTGCY

SEQ ID NO: 17 - protein, 14 aa, synthetic construct
  REGION 1..14: note = Synthetically generated peptide
  MOD_RES 1: note = allylglycine
  MOD_RES 4: note = 4-fluorophenylalanine
  MOD_RES 6: note = allylglycine
  MOD_RES 14: note = 4-fluorophenylalanine
  MOD_RES 14: note = AMIDATION
GCEFCGNPACTGCF

SEQ ID NO: 18 - protein, 14 aa, synthetic construct
  REGION 1..14: note = Synthetically generated peptide
  MOD_RES 5: note = allylglycine
  MOD_RES 13: note = allylglycine
  MOD_RES 14: note = AMIDATION
CCELGCNPACTGGY

SEQ ID NO: 19 - protein, 13 aa, synthetic construct
  REGION 1..13: note = Synthetically generated peptide
  MOD_RES 1: note = allylglycine
  MOD_RES 6: note = allylglycine
  MOD_RES 13: note = AMIDATION
GCEFCGNPACTGC

SEQ ID NO: 20 - protein, 14 aa, synthetic construct
  REGION 1..14: note = Synthetically generated peptide
  MOD_RES 1: note = allylglycine
  MOD_RES 6: note = allylglycine
  MOD_RES 14: note = 4-fluorophenylalanine
  MOD_RES 14: note = AMIDATION
GCEFCGNPACTGCF

SEQ ID NO: 21 - protein, 14 aa, synthetic construct
  REGION 1..14: note = Synthetically generated peptide
  MOD_RES 4: note = 4-fluorophenylalanine
  MOD_RES 14: note = AMIDATION
CCEFCCNPACTGCY

SEQ ID NO: 22 - protein, 14 aa, synthetic construct
  REGION 1..14: note = Synthetically generated peptide
  MOD_RES 4: note = 4-fluorophenylalanine
  MOD_RES 14: note = 4-fluorophenylalanine
  MOD_RES 14: note = AMIDATION
CCEFCCNPACTGCF

SEQ ID NO: 23 - protein, 14 aa, synthetic construct
  REGION 1..14: note = Synthetically generated peptide
  MOD_RES 1: note = allylglycine
  MOD_RES 6: note = allylglycine
  MOD_RES 14: note = 4-fluorophenylalanine
  MOD_RES 14: note = AMIDATION
GCELCGNPACTGCF

SEQ ID NO: 24 - protein, 15 aa, synthetic construct
  REGION 1..15: note = Synthetically generated peptide
  MOD_RES 1: note = 4-fluorophenylalanine
  MOD_RES 2: note = allylglycine
  MOD_RES 7: note = allylglycine
  MOD_RES 15: note = 4-fluorophenylalanine
  MOD_RES 15: note = AMIDATION
FGCELCGNPACTGCF

SEQ ID NO: 25 - protein, 14 aa, synthetic construct
  REGION 1..14: note = Synthetically generated peptide
  MOD_RES 1: note = allylglycine
  MOD_RES 6: note = allylglycine
  MOD_RES 14: note = AMIDATION
GCELCGNPACAGCY

SEQ ID NO: 26 - protein, 14 aa, synthetic construct
  REGION 1..14: note = Synthetically generated peptide
  MOD_RES 1: note = allylglycine
  MOD_RES 6: note = allylglycine
  MOD_RES 14: note = AMIDATION
GCELCGNPACYGCY

SEQ ID NO: 27 - protein, 14 aa, synthetic construct
  REGION 1..14: note = Synthetically generated peptide
  MOD_RES 1: note = allylglycine
  MOD_RES 6: note = allylglycine
  MOD_RES 14: note = AMIDATION
GCELCGNPACFGCY

SEQ ID NO: 28 - protein, 14 aa, synthetic construct
  REGION 1..14: note = Synthetically generated peptide
  MOD_RES 1: note = allylglycine
  MOD_RES 6: note = allylglycine
  MOD_RES 11: note = 4-fluorophenylalanine
  MOD_RES 14: note = AMIDATION
GCELCGNPACFGCY

SEQ ID NO: 29 - protein, 14 aa, synthetic construct
  REGION 1..14: note = Synthetically generated peptide
  MOD_RES 1: note = allylglycine
  MOD_RES 6: note = allylglycine
  MOD_RES 14: note = AMIDATION
GCELCGNPACTGCL

SEQ ID NO: 30 - protein, 14 aa, synthetic construct
  REGION 1..14: note = Synthetically generated peptide
  MOD_RES 1: note = allylglycine
  MOD_RES 6: note = allylglycine
  MOD_RES 14: note = cyclohexylalanine
  MOD_RES 14: note = AMIDATION
GCELCGNPACTGCA

SEQ ID NO: 31 - protein, 14 aa, synthetic construct
  REGION 1..14: note = Synthetically generated peptide
  MOD_RES 1: note = allylglycine
  MOD_RES 4: note = cyclohexylalanine
  MOD_RES 6: note = allylglycine
  MOD_RES 14: note = AMIDATION
GCEACGNPACTGCY

SEQ ID NO: 32 - protein, 14 aa, synthetic construct
  REGION 1..14: note = Synthetically generated peptide
  MOD_RES 1: note = allylglycine
  MOD_RES 6: note = allylglycine
  MOD_RES 14: note = D-Tyr
  MOD_RES 14: note = AMIDATION
GCELCGNPACTGCY

SEQ ID NO: 33 - protein, 14 aa, synthetic construct
  MOD_RES 1: note = allylglycine
  MOD_RES 2: note = penicillamine
  MOD_RES 6: note = allylglycine
  MOD_RES 10: note = penicillamine
  MOD_RES 14: note = AMIDATION
  REGION 1..14: note = Synthetically generated peptide
GXELCGNPAXTGCY

SEQ ID NO: 34 - protein, 15 aa, synthetic construct
  REGION 1..15: note = Synthetically generated peptide
  MOD_RES 1: note = allylglycine
  MOD_RES 6: note = allylglycine
  MOD_RES 15: note = AMIDATION
GCELCGNPACTGCLN

SEQ ID NO: 35 - protein, 15 aa, synthetic construct
  REGION 1..15: note = Synthetically generated peptide
  MOD_RES 1: note = allylglycine
  MOD_RES 6: note = allylglycine
  MOD_RES 15: note = AMIDATION
GCELCGNPACTGCYN

SEQ ID NO: 36 - protein, 15 aa, synthetic construct
  REGION 1..15: note = Synthetically generated peptide
  MOD_RES 2: note = allylglycine
  MOD_RES 7: note = allylglycine
  MOD_RES 15: note = AMIDATION
DGCELCGNPACTGCY

SEQ ID NO: 37 - protein, 16 aa, synthetic construct
  REGION 1..16: note = Synthetically generated peptide
  MOD_RES 3: note = allylglycine
  MOD_RES 8: note = allylglycine
  MOD_RES 16: note = AMIDATION
IDGCELCGNPACTGCY

SEQ ID NO: 38 - protein, 14 aa, synthetic construct
  REGION 1..14: note = Synthetically generated peptide
  MOD_RES 1: note = allylglycine
  MOD_RES 6: note = allylglycine
  MOD_RES 8: note = hydroxyproline
  MOD_RES 14: note = AMIDATION
GCELCGNPACTGCY

SEQ ID NO: 39 - protein, 13 aa, synthetic construct
  MOD_RES 1: note = allylglycine
  MOD_RES 6: note = allylglycine
  MOD_RES 8: note = hydroxyproline
  MOD_RES 13: note = AMIDATION
GCELCGNPACTGC

SEQ ID NO: 40 - protein, 14 aa, synthetic construct
  REGION 1..14: note = Synthetically generated peptide
  MOD_RES 1: note = allylglycine
  MOD_RES 5: note = penicillamine
  MOD_RES 6: note = allylglycine
  MOD_RES 13: note = penicillamine
  MOD_RES 14: note = AMIDATION
GCELXGNPACTGXY

SEQ ID NO: 41 - protein, 14 aa, synthetic construct
  MOD_RES 1: note = allylglycine
  MOD_RES 5: note = penicillamine
  MOD_RES 6: note = allylglycine
  MOD_RES 13: note = penicillamine
  MOD_RES 14: note = AMIDATION
GCELXGNPACTGXY

SEQ ID NO: 42 - protein, 14 aa, synthetic construct
  REGION 1..14: note = Synthetically generated peptide
  MOD_RES 1: note = allylglycine
  MOD_RES 6: note = allylglycine
  MOD_RES 14: note = N-methyl tyrosine
  MOD_RES 14: note = AMIDATION
GCELCGNPACYGCY

SEQ ID NO: 43 - protein, 13 aa, synthetic construct
  REGION 1..13: note = Synthetically generated peptide
  MOD_RES 1: note = allylglycine
  MOD_RES 6: note = allylglycine
GCELCGNPACYGC

SEQ ID NO: 44 - protein, 14 aa, synthetic construct
  REGION 1..14: note = Synthetically generated peptide
  MOD_RES 1: note = allylglycine
  MOD_RES 6: note = allylglycine
  MOD_RES 11: note = cyclohexylalanine
  MOD_RES 14: note = AMIDATION
GCELCGNPACAGCY

SEQ ID NO: 45 - protein, 14 aa, synthetic construct
  REGION 1..14: note = Synthetically generated peptide
  MOD_RES 1: note = allylglycine
  MOD_RES 6: note = allylglycine
  MOD_RES 14: note = AMIDATION
GCELCGNLACTGCY

SEQ ID NO: 46 - protein, 14 aa, synthetic construct
  MOD_RES 1: note = allylglycine
  MOD_RES 6: note = allylglycine
  MOD_RES 8: note = sarcosine
  MOD_RES 14: note = AMIDATION
  REGION 1..14: note = Synthetically generated peptide
GCELCGNXACTGCY

SEQ ID NO: 47 - protein, 14 aa, synthetic construct
  REGION 1..14: note = Synthetically generated peptide
  MOD_RES 1: note = allylglycine
  MOD_RES 6: note = allylglycine
  MOD_RES 14: note = AMIDATION
GCELCGNVACTGCY

SEQ ID NO: 48 - protein, 14 aa, synthetic construct
  REGION 1..14: note = Synthetically generated peptide
  MOD_RES 1: note = allylglycine
  MOD_RES 6: note = allylglycine
  MOD_RES 14: note = N-methyl tyrosine
  MOD_RES 14: note = AMIDATION
GCELCGNPACYGCY

SEQ ID NO: 49 - protein, 13 aa, synthetic construct
  REGION 1..13: note = Synthetically generated peptide
  MOD_RES 1: note = allylglycine
  MOD_RES 6: note = allylglycine
GCELCGLPACYGC

SEQ ID NO: 50 - protein, 13 aa, synthetic construct
  REGION 1..13: note = Synthetically generated peptide
  MOD_RES 1: note = allylglycine
  MOD_RES 6: note = allylglycine
GCDLCGNPACYGC

SEQ ID NO: 51 - protein, 13 aa, synthetic construct
  REGION 1..13: note = Synthetically generated peptide
  MOD_RES 1: note = allylglycine
  MOD_RES 6: note = allylglycine
GCSLCGNPACYGC

SEQ ID NO: 52 - protein, 13 aa, synthetic construct
  REGION 1..13: note = Synthetically generated peptide
  MOD_RES 1: note = allylglycine
  MOD_RES 6: note = allylglycine
GCTLCGNPACYGC

SEQ ID NO: 53 - protein, 14 aa, synthetic construct
  REGION 1..14: note = Synthetically generated peptide
  MOD_RES 1: note = allylglycine with a reduced dicarba bond
  MOD_RES 6: note = allylglycine with a reduced dicarba bond
  MOD_RES 14: note = AMIDATION
GCELCGNPACTGCY

SEQ ID NO: 54 - protein, 13 aa, synthetic construct
  REGION 1..13: note = Synthetically generated peptide
  MOD_RES 1: note = allylglycine
  MOD_RES 6: note = allylglycine
GCQLCGNPACYGC

SEQ ID NO: 55 - protein, 14 aa, synthetic construct
  REGION 1..14: note = Synthetically generated peptide
  MOD_RES 1: note = allylglycine
  MOD_RES 6: note = allylglycine
  MOD_RES 14: note = AMIDATION
GCELCGLPACTGCY

SEQ ID NO: 56 - protein, 13 aa, synthetic construct
  REGION 1..13: note = Synthetically generated peptide
  MOD_RES 1: note = N-terminus capped with pentenoic acid
  MOD_RES 5: note = allylglycine
  MOD_RES 13: note = AMIDATION
CELCGNPACYGCY

SEQ ID NO: 57 - protein, 13 aa, synthetic construct
  REGION 1..13: note = Synthetically generated peptide
  MOD_RES 6: note = allylglycine
  MOD_RES 13: note = allylglycine
CCELCGNPACYGG

SEQ ID NO: 58 - protein, 12 aa, synthetic construct
  REGION 1..12: note = Synthetically generated peptide
  MOD_RES 1: note = N-terminus capped with pentenoic acid
  MOD_RES 5: note = allylglycine
CELCGNPACYGC

SEQ ID NO: 59 - protein, 13 aa, synthetic construct
  REGION 1..13: note = Synthetically generated peptide
  MOD_RES 1: note = allylglycine with a reduced dicarba bond
  MOD_RES 6: note = allylglycine with a reduced dicarba bond
GCELCGNPACYGC

SEQ ID NO: 60 - protein, 14 aa, synthetic construct
  REGION 1..14: note = Synthetically generated peptide
  MOD_RES 1: note = allylglycine
  MOD_RES 6: note = allylglycine
  MOD_RES 14: note = AMIDATION
GCELCGNVACYGCY

SEQ ID NO: 61 - protein, 13 aa, synthetic construct
  REGION 1..13: note = Synthetically generated peptide
  MOD_RES 1: note = allylglycine
  MOD_RES 6: note = allylglycine
GCELCGNVACYGC

SEQ ID NO: 62 - protein, 14 aa, synthetic construct
  MOD_RES 2: note = cystathionine
  MOD_RES 14: note = AMIDATION
  REGION 1..14: note = Synthetically generated peptide
CXELCCNPACYGCY

SEQ ID NO: 63 - protein, 14 aa, synthetic construct
  REGION 1..14: note = Synthetically generated peptide
  MOD_RES 1: note = cystathionine
  MOD_RES 14: note = AMIDATION
XCELCCNPACYGCY

SEQ ID NO: 64 - protein, 14 aa, synthetic construct
  MOD_RES 1: note = cystathionine
  REGION 1..14: note = Synthetically generated peptide
XCELCCNPACYGCY

SEQ ID NO: 65 - protein, 14 aa, synthetic construct
  MOD_RES 2: note = cystathionine
  MOD_RES 14: note = AMIDATION
  REGION 1..14: note = Synthetically generated peptide
CXELCCNVACYGCY

SEQ ID NO: 66 - protein, 14 aa, synthetic construct
  REGION 1..14: note = Synthetically generated peptide
  MOD_RES 2: note = cystathionine
  MOD_RES 14: note = AMIDATION
CXELCCNVACTGCY

SEQ ID NO: 67 - protein, 14 aa, synthetic construct
  REGION 1..14: note = Synthetically generated peptide
  MOD_RES 1: note = acetylated N-terminus
  MOD_RES 2: note = cystathionine
  MOD_RES 14: note = AMIDATION
CXELCCNVACTGCY

SEQ ID NO: 68 - protein, 14 aa, synthetic construct
  REGION 1..14: note = Synthetically generated peptide
  MOD_RES 1: note = acetylated N-terminus
  MOD_RES 2: note = cystathionine
  MOD_RES 14: note = C-terminus is amidated
CXELCCNPACYGCY

SEQ ID NO: 69 - protein, 13 aa, synthetic construct
  REGION 1..13: note = Synthetically generated peptide
  MOD_RES 1: note = acetylated N-terminus
  MOD_RES 2: note = cystathionine
CXELCCNVACYGC

SEQ ID NO: 70 - protein, 13 aa, synthetic construct
  REGION 1..13: note = Synthetically generated peptide
  MOD_RES 2: note = cystathionine
CXELCCNVACYGC

SEQ ID NO: 71 - protein, 13 aa, synthetic construct
  REGION 1..13: note = Synthetically generated peptide
  MOD_RES 1: note = acetylated N-terminus
  MOD_RES 2: note = cystathionine
CXELCCNPACYGC

SEQ ID NO: 72 - protein, 12 aa, synthetic construct
  REGION 1..12: note = Synthetically generated peptide
  MOD_RES 9: note = cystathionine
CCELCNVAXYGC

SEQ ID NO: 73 - protein, 14 aa, synthetic construct
  REGION 1..14: note = Synthetically generated peptide
  MOD_RES 10: note = cystathionine
  MOD_RES 14: note = AMIDATION
CCELCCNVAXYGCY

SEQ ID NO: 74 - protein, 14 aa, synthetic construct
  REGION 1..14: note = Synthetically generated peptide
  MOD_RES 10: note = cystathionine
  MOD_RES 14: note = AMIDATION
CCELCCNVAXTGCY

SEQ ID NO: 75 - protein, 13 aa, synthetic construct
  REGION 1..13: note = Synthetically generated peptide
  MOD_RES 6: note = cystathionine
CCELCXNVACYGC

SEQ ID NO: 76 - protein, 14 aa, synthetic construct
  REGION 1..14: note = Synthetically generated peptide
  MOD_RES 6: note = cystathionine
  MOD_RES 14: note = AMIDATION
CCELCXNVACYGCY

SEQ ID NO: 77 - protein, 14 aa, synthetic construct
  REGION 1..14: note = Synthetically generated peptide
  MOD_RES 6: note = cystathionine
  MOD_RES 14: note = AMIDATION
CCELCXNVACTGCY

SEQ ID NO: 78 - protein, 13 aa, synthetic construct
  REGION 1..13: note = Synthetically generated peptide
  MOD_RES 1: note = N-terminus capped with 4-Mepip (1-methyl-piperidine-4-carboxylic acid)
  MOD_RES 2: note = cystathionine
CXELCCNVACYGC

SEQ ID NO: 79 - protein, 13 aa, synthetic construct
  REGION 1..13: note = Synthetically generated peptide
  MOD_RES 2: note = cystathionine
CXELCCNVACTGC

SEQ ID NO: 80 - protein, 13 aa, synthetic construct
  REGION 1..13: note = Synthetically generated peptide
  MOD_RES 2: note = cystathionine
  MOD_RES 13: note = AMIDATION
CXELCCNVACTGC

SEQ ID NO: 81 - protein, 13 aa, synthetic construct
  REGION 1..13: note = Synthetically generated peptide
  MOD_RES 2: note = cystathionine
CXELCCNVACFGC

SEQ ID NO: 82 - protein, 14 aa, synthetic construct
  REGION 1..14: note = Synthetically generated peptide
  MOD_RES 1: note = allylglycine
  MOD_RES 2: note = allylglycine
  MOD_RES 5: note = allylglycine
  MOD_RES 6: note = allylglycine
  MOD_RES 10: note = allylglycine
  MOD_RES 13: note = allylglycine
GGELGGNPAGTGGY

SEQ ID NO: 83 - protein, 14 aa, synthetic construct
  REGION 1..14: note = Synthetically generated peptide
  MOD_RES 2: note = cystathionine
  MOD_RES 14: note = AMIDATION
CXELCCNAACTGCY

SEQ ID NO: 84 - protein, 14 aa, synthetic construct
  REGION 1..14: note = Synthetically generated peptide
  MOD_RES 6: note = di-aminopropionic acid
  MOD_RES 14: note = AMIDATION
DCELCXNVACTGCY

SEQ ID NO: 85 - protein, 14 aa, synthetic construct
  REGION 1..14: note = Synthetically generated peptide
  MOD_RES 2: note = cystathionine
  MOD_RES 14: note = AMIDATION
CXELCCNVACSGCY

SEQ ID NO: 86 - protein, 13 aa, synthetic construct
  REGION 1..13: note = Synthetically generated peptide
  MOD_RES 1: note = cystathionine
  MOD_RES 5: note = allylglycine
  MOD_RES 13: note = AMIDATION
XELCGNVACTGCY

SEQ ID NO: 87 - protein, 14 aa, synthetic construct
  REGION 1..14: note = Synthetically generated peptide
  MOD_RES 1: note = allylglycine
  MOD_RES 6: note = allylglycine
  MOD_RES 14: note = AMIDATION
GCELCGNVACTGCY

SEQ ID NO: 88 - protein, 14 aa, synthetic construct
  REGION 1..14: note = Synthetically generated peptide
  MOD_RES 1: note = allylglycine
  MOD_RES 6: note = allylglycine
  MOD_RES 14: note = AMIDATION
GCELCGNPACTGCY

SEQ ID NO: 89 - protein, 13 aa, synthetic construct
  REGION 1..13: note = Synthetically generated peptide
  MOD_RES 1: note = cystathionine
XCELCCNVACTGC

SEQ ID NO: 90 - protein, 14 aa, synthetic construct
  REGION 1..14: note = Synthetically generated peptide
  MOD_RES 1: note = cystathionine
  MOD_RES 14: note = AMIDATION
XCELCCNVACTGCY

SEQ ID NO: 91 - protein, 14 aa, synthetic construct
  REGION 1..14: note = Synthetically generated peptide
  MOD_RES 1: note = cystathionine
  MOD_RES 14: note = AMIDATION
XCELCCNVACYGCY

SEQ ID NO: 92 - protein, 13 aa, synthetic construct
  REGION 1..13: note = Synthetically generated peptide
  VARIANT 10: note = Xaa can be any naturally occurring amino acid
  MOD_RES 13: note = cystathionine
CCELCCNVAXYGC

SEQ ID NO: 93 - protein, 21 aa, synthetic construct
  REGION 1..21: note = Synthetically generated peptide
  VARIANT 1: note = Xaa1 is glutamic acid wherein the side chain carboxylic acid forms the peptide linkage or is absent
  VARIANT 2: note = Xaa2 is lysine wherein the side chain amine forms the peptide linkage or is absent
  VARIANT 3: note = Xaa3 is Asn or is absent
  VARIANT 4: note = Xaa4 is Ser or is absent
  VARIANT 5: note = Xaa5 is Ser, Asn, Ile, or is absent
  VARIANT 6: note = Xaa6 is Tyr, Asp, 4-fluorophenylalanine ((4-F)Phe), or is absent
  VARIANT 7: note = Xaa7 is Cys, cystathionine (Cth), allylglycine (Ag), allylglycine with a reduced dicarba bond (Hag), or Asp
  VARIANT 8: note = Xaa8 is Cys, cystathionine (Cth), penicillamine (Pen), or allylglycine (Ag)
  VARIANT 9: note = Xaa9 is Glu, Asp, Ser, Thr, or Gln
  VARIANT 10: note = Xaa10 is Leu, cyclohexylalanine (Cha), Phe, or 4-fluorophenylalanine ((4-F)Phe)
  VARIANT 12: note = Xaa12 is Cys, allylglycine (Ag), Hag, or Val
  VARIANT 14: note = Xaa14 is Pro, Val, sarcosine (Sar), Leu, or Hydroxyproline (OH-Pro)
  VARIANT 17: note = Xaa17 is Tyr, Thr, cyclohexylalanine (Cha), 4-fluorophenylalanine ((4-F)Phe), Phe, or Ala
  VARIANT 20: note = Xaa20 is Tyr, Leu, 4-fluorophenylalanine ((4-F)Phe), cyclohexylalanine (Cha), D-Tyr, N-Methyl Tyr (Nme-Tyr) or is absent
  VARIANT 21: note = Xaa21 is absent or Asnt
XXXXXXXXXXCXNXACXGCXX

SEQ ID NO: 94 - protein, 14 aa, synthetic construct
  REGION 1..14: note = Synthetically generated peptide
CCELCCNPACTGCY

(19) United States
(12) Patent Application Publication
(10) Pub. No.: US 2024/0199697 A1
(43) Pub. Date: Jun. 20, 2024

(54) Compositions for Colon Cleansing and the Treatment of Gastrointestinal Disorders

(71) Applicant: Ironwood Pharmaceuticals, Inc., Boston, MA (US)

(72) Inventors: Mark G. Currie, Boston, MA (US); Robert Solinga, Brookline, MA (US); Christopher Leitheiser, Arlington, MA (US)

(21) Appl. No.: 18/424,114

(22) Filed: Jan. 26, 2024

Related U.S. Application Data
(63) Continuation of application No. 18/059,260, filed on Nov. 28, 2022, now abandoned.
Application No. 18/059,260 is a continuation of application No. 17/716,627, filed on Apr. 8, 2022, now abandoned.
Application No. 17/716,627 is a continuation of application No. 17/387,458, filed on Jul. 28, 2021, now abandoned.
Application No. 17/387,458 is a continuation of application No. 16/953,038, filed on Nov. 19, 2020, now abandoned.
Application No. 16/953,038 is a continuation of application No. 16/807,274, filed on Mar. 3, 2020, now abandoned.
Application No. 16/807,274 is a continuation of application No. 15/570,838, filed on Oct. 31, 2017, now Pat. No. 10,618,938.
(60) Provisional application No. 62/156,077, filed on May 1, 2015.

(51) Int. Cl. C07K 7/08; A61K 9/00; A61K 38/00; A61K 38/10; A61K 38/12; A61K 45/06; A61P 1/10; A61P 35/00

(52) U.S. Cl. CPC C07K 7/08; A61K 9/0031; A61K 9/0053; A61K 38/10; A61K 38/12; A61K 45/06; A61P 1/10; A61P 35/00; A61K 38/00

(57) ABSTRACT

The present invention provides peptides and compositions that are useful for the treatment of gastrointestinal disorders or for colon cleansing. The present invention also provides compositions and methods of treating gastrointestinal disorders and pharmaceutical compositions for accomplishing the same. In some embodiments, these pharmaceutical compositions include oral dosage forms.

### CLAIM OF PRIORITY

This application is a continuation of, and claims priority under 35 U.S.C. § 120, to U.S. patent application Ser. No. 18/059,260 filed Nov. 28, 2022, which is a continuation of U.S. application Ser. No. 17/716,627 filed Apr. 8, 2022, which is a continuation of U.S. application Ser. No. 17/387,458 filed Jul. 28, 2021, which is a continuation of U.S. patent application Ser. No. 16/953,038 filed Nov. 19, 2020, which is a continuation of U.S. patent application Ser. No. 16/807,274 filed Mar. 3, 2020, which is a continuation of patent application Ser. No. 15/570,838 filed Oct. 31, 2017, which is the United States National Phase filing of PCT/US2016/030061, filed Apr. 29, 2016. This application also claims priority under 35 USC § 119(e) to U.S. Provisional Patent Application Ser. No. 62/156,077 filed on May 1, 2015. The entire contents of the aforementioned applications are incorporated herein by reference.

### FIELD OF THE INVENTION

This invention relates to peptides, compositions and methods for colon cleansing and treatments of disorders of the gastrointestinal tract and other visceral organs.

### SEQUENCE LISTING

This application incorporates by reference in its entirety the Sequence Listing entitled “223355-519461.xml” (221,049 bytes) which was created Jan. 9, 2024 and filed electronically herewith.

### BACKGROUND

Approximately 15 million colonoscopies are performed annually in the United States, all of which require adequate colon preparation. Inadequate bowel preparation has been reported in about 20% of colonoscopies. Inadequate bowel preparation may result in lower adenoma detection, longer procedural time, and shorter intervals between examinations. As a result, there is a need for safe, effective, and well tolerated colon preparations that allow a broad population of patients to reliably and effectively under go colonoscopies in order to reduce the risk of colon cancer.

### SUMMARY

The present invention features peptides, compositions, and related methods for colon cleansing treatments as well as other conditions and disorders are described herein. In one embodiment, the peptides may be used to prepare subjects for colonoscopy treatment. In some embodiments, the peptides or pharmaceutically acceptable salts may be used to prepare subjects for surgery, such as bowel surgery. In other embodiments, the peptides may be used to treat colon cancer, Hereditary Nonpolyposis Colorectal Cancer (HNPCC), i.e. Lynch syndrome, gastroparesis (GP), polyps, pain, general abdominal pain, post-operative ileus, opioid-induced constipation, functional dyspepsia, diverticular disease including including but not limited to SUDD (symptomatic uncomplicated diverticular disease) and SCAD (segmental colitis associated with diverticulosis), diverticulosis, diarrhea-predominant irritable bowel syndrome, pain associated with irritable bowel syndrom (IBS), ulcerative colitis, ulcerative proctitis, Crohn's Disease, inflammatory bowel disease (IBD), chronic or acute radiation protopathy, rectal pain, chronic proctalgia, proctalgia fugax, anal pain, chronic anal fissure, post-operative anal pain, overactive bladder syndrome, stress incontinence, interstitial cystitis, bladder pain syndrome, colorectal cancer, pain associated with cancer, general pelvic pain, endometriosis, orchialgia, chronic prostatitis, prostatodynia, vulvodynia, urethral syndrome, penile pain, perianal pain and other gastrointestinal and visceral disorders.

One aspect of the present invention provides a peptide or a pharmaceutically acceptable salt thereof, wherein the peptide comprises the amino acid sequence:
Xaa₁ Xaa₂ Xaa₃ Xaa₄ Xaa₅ Xaa₆ Xaa₇ Xaa₈ Xaa₉ Xaa₁₀ Xaa₁₁ Xaa₁₂ Xaa₁₃ Xaa₁₄ Ala₁₅ Xaa₁₆ Xaa₁₇ Gly₁₈ Xaa₁₉ Xaa₂₀ Xaa₂₁ (SEQ ID NO: 1), or a pharmaceutically acceptable salt thereof; wherein
Xaa₁ is BE or is absent;
Xaa₂ is BK, Asn or is absent;
Xaa₃ is Asn, Ser or is absent;
Xaa₄ is Ser or is absent;
Xaa₅ is Ser, Asn, Ile, BE or is absent;
Xaa₆ is Tyr, Asp, 4-fluorophenylalanine ((4-F)Phe), BK or is absent;
Xaa₇ is Cys, cystathionine (Cth), allylglycine (Ag), Hag, or Asp;
Xaa₈ is Cys, cystathionine (Cth), penicillamine (Pen), or allylglycine (Ag);
Xaa₉ is Glu, Asp, Ser, Thr, or Gln;
Xaa₁₀ is Leu, cyclohexylalanine (Cha), Phe, or 4-fluorophenylalanine ((4-F)Phe);
Xaa₁₁ is Cys, Ag, or penicillamine (Pen);
Xaa₁₂ is Cys, allylglycine (Ag), Hag, Cth, Dpr, or Val;
Xaa₁₃ is Asn or Leu;
Xaa₁₄ is Pro, Val, sarcosine (Sar), Leu, or Hydroxyproline (OH-Pro);
Xaa₁₆ is Cys, Ag, Pen or Cth;
Xaa₁₇ is Tyr, Thr, cyclohexylalanine (Cha), 4-fluorophenylalanine ((4-F)Phe), Phe, Ser, or Ala;
Xaa₁₉ is Cys, Ag or Pen;
Xaa₂₀ is Tyr, Leu, 4-fluorophenylalanine ((4-F)Phe), cyclohexylalanine (Cha), D-Tyr, N-Methyl Tyr (Nme-Tyr) or is absent;
Xaa₂₁ is absent or Asn;
wherein at least one Xaa is BE, BK, (4-F)Phe, Cth, Ag, Hag, Pen, Cha, Sar, Dpr, or OH-Pro; and
   wherein the peptide contains a covalent bond between Xaa₇ and Xaa₁₂, Xaa₈ and Xaa₁₆, and Xaa₁₁ and Xaa₁₉.

A second aspect of the present invention provides pharmaceutical compositions comprising a peptide of the present invention.

A third aspect of the present invention provides methods for treating a gastrointestinal disorder, which include administering a pharmaceutical composition according to the present invention. The pharmaceutical compositions according to the present invention may be used as a preparation for a colonoscopy, or treatment of gastrointestinal disorders and pain. In some embodiments, the composition is a solid, oral composition.

The details of one or more embodiments of the invention are set forth in the accompanying description.

### BRIEF DESCRIPTION OF THE FIGURES

FIG. 1 illustrates the activity and stability results for representative peptides.

FIG. 2 illustrates the results of a cGMP accumulation in T84 cell assay for analysis of GC-C activity for SEQ ID NOs: 3-7.

FIG. 3 illustrates the results of a cGMP accumulation in T84 cell assay for analysis of GC-C activity for SEQ ID NOs 8-10.

FIG. 4 presents the results of a cGMP accumulation in T84 cell assay for analysis of GC-C activity for SEQ ID NOs 12-13 and 23-27.

FIG. 5 illustrates the results of a cGMP accumulation in T84 cell assay for analysis of GC-C activity for SEQ ID NOs 28-34.

FIG. 6 illustrates the results of a cGMP accumulation in T84 cell assay for analysis of GC-C activity for SEQ ID NOs 35-37, 39 and 44.

FIG. 7 illustrates the results of a cGMP accumulation in T84 cell assay for analysis of GC-C activity for SEQ ID NOs 2 and 40-42.

FIG. 8 illustrates the results of a cGMP accumulation in T84 cell assay for analysis of GC-C activity for SEQ ID NO 43.

FIG. 9 illustrates the results of a cGMP accumulation in T84 cell assay for analysis of GC-C activity for SEQ ID NOs 44-47.

FIG. 10 illustrates the results of a cGMP accumulation in T84 cell assay for analysis of GC-C activity for SEQ ID NOs 45, 48 and 51-52.

FIG. 11 illustrates the results of a cGMP accumulation in T84 cell assay for analysis of GC-C activity for SEQ ID NOs 12, 13, 27, 47 and 53.

FIG. 12 illustrates the results of a cGMP accumulation in T84 cell assay for analysis of GC-C activity for SEQ ID NO 47 with an original test, a repetition of the original test, and a second weighing.

FIG. 13 illustrates the results of a cGMP accumulation in T84 cell assay for analysis of GC-C activity for SEQ ID NOs 54-59.

FIG. 14 illustrates the results of a cGMP accumulation in T84 cell assay for analysis of GC-C activity for SEQ ID NOs 60-64.

FIG. 15 illustrates the results of an in vivo rat duodenal loop assay for SEQ ID NOs 62 and 47.

FIG. 16 illustrates the results of a rat duodenal loop volume test for SEQ ID NOs 62 and 47.

FIG. 17 illustrates the results of a mouse gut transit (mGIT) assay for SEQ ID NOs 2 and 47 in comparison with the ST Core.

FIG. 18 illustrates the results of an in vivo ligated rat loop assay for SEQ ID NOs 67 and 69.

FIG. 19 illustrates the results of an in vivo ligated rat loop volume test for SEQ ID NOs 67 and 69.

FIG. 20 presents the results of an in vitro rat intestinal fluid (RIF) assay for SEQ ID NOs 2, 26, 43, 47 and 53.

FIG. 21 presents the results of an in vitro RIF assay for SEQ ID NOs 60-66.

FIG. 22 presents the results of an in vivo RIF assay for SEQ ID NOs 47, 62, 67 and 69.

FIG. 23 presents the results of differing assays performed on representative peptides.

FIG. 24 presents the results of a cGMP accumulation in T84 cell assay for SEQ ID NOs 14-22.

FIG. 25 illustrates the results of a mouse gut transit assay for SEQ ID NOs 2, 7 and 11.

FIG. 26 illustrates the results of a mouse gut transit assay for SEQ ID NO 47.

FIG. 27 illustrates the results of a mouse gut transit assay for SEQ ID NOs 62, 67 and 69.

These figures are provided by way of example and are not intended to limit the scope of the present invention.

### DETAILED DESCRIPTION

Guanylate cyclase C (GC-C) is a transmembrane receptor that is located on the apical surface of epithelial cells in the stomach and intestine. The receptor has an extracellular ligand-binding domain, a single transmembrane region and a C-terminal guanylyl cyclase domain. When a ligand binds to the extracellular domain of GC-C, the intracellular catalytic domain catalyzes the production of cGMP from GTP. In vivo, this increase in intracellular cGMP initiates a cascade of events that leads to increased secretion of chloride and bicarbonate into the intestinal lumen, increased luminal pH, decreased luminal sodium absorption, increased fluid secretion, and acceleration of intestinal transit. cGMP, which is secreted bidirectionally from the epithelium into the mucosa and lumen, has also been shown to dampen afferent C fiber firing, suggesting a potential mechanism for the observed analgesic effects of GC-C agonists on visceral pain.

Linaclotide, a peptide GC-C agonist that is orally administered and currently approved in the United States for the treatment of irritable bowel syndrome with constipation (IBS-c) and chronic idiopatheic constipation (CIC), has numerous effects on lower GI physiology including: (1) reduced visceral pain, (2) reduced bloating, and (3) increased GI transit, which can lead to increased stool frequency and improved stool consistency. Orally administered linaclotide acts locally by activating GC-C receptors at the luminal surface. Thus, the results from clinical trials of linaclotide, as well as preclinical studies that have been done with linaclotide and related peptides, suggest that GC-C peptide agonists may be used therapeutically. The peptides described and claimed herein may bind and activate the GC-C receptor or may be characterized as GC-C peptide agonists.

### Definitions

As used herein, C12 is C12 alkyl carboxylic acid, C14 is C14 alkyl carboxylic acid, C16 is C16 alkyl carboxylic acid, C18 is C18 alkyl carboxylic acid, (4-F)Phe is 4-fluorophenylalanine, Cth is cystathionine, Ag is allylglycine, Hag is allylglycine with a reduced dicarba bond, Pent is pentenoic acid, Pen is penicillamine, Cha is cyclohexylalanine, Sar is sarcosine, OH-Pro is hydroxyproline, Nme-Tyr is N-methyl tyrosine, 4-Mepip is 1-methyl-piperidine-4-carboxylic acid, Dpr is di-aminopropionic acid, BE is glutamic acid wherein the side chain carboxylic acid forms the peptide linkage, and BK is lysine wherein the side chain amine forms the peptide linkage. An example of a BE BK bonding pattern would be:

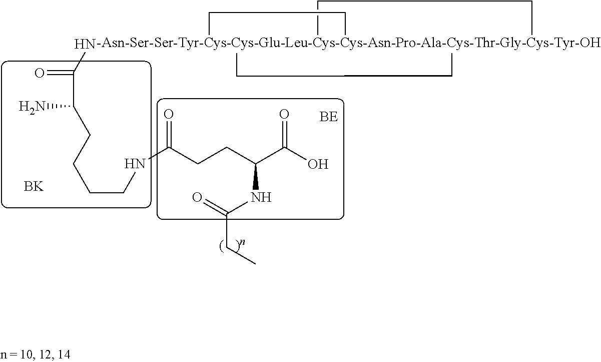

Additional examples of peptide bonding include without limitation:

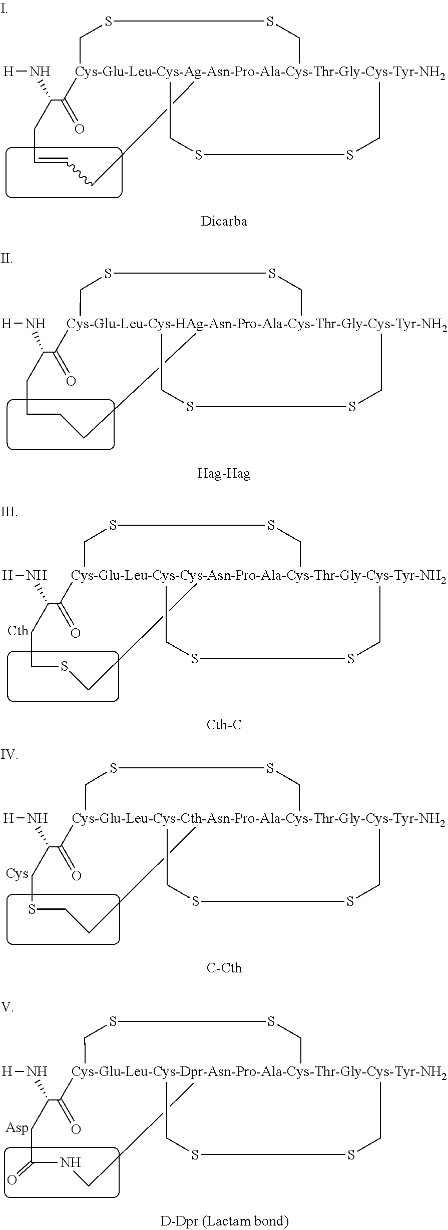

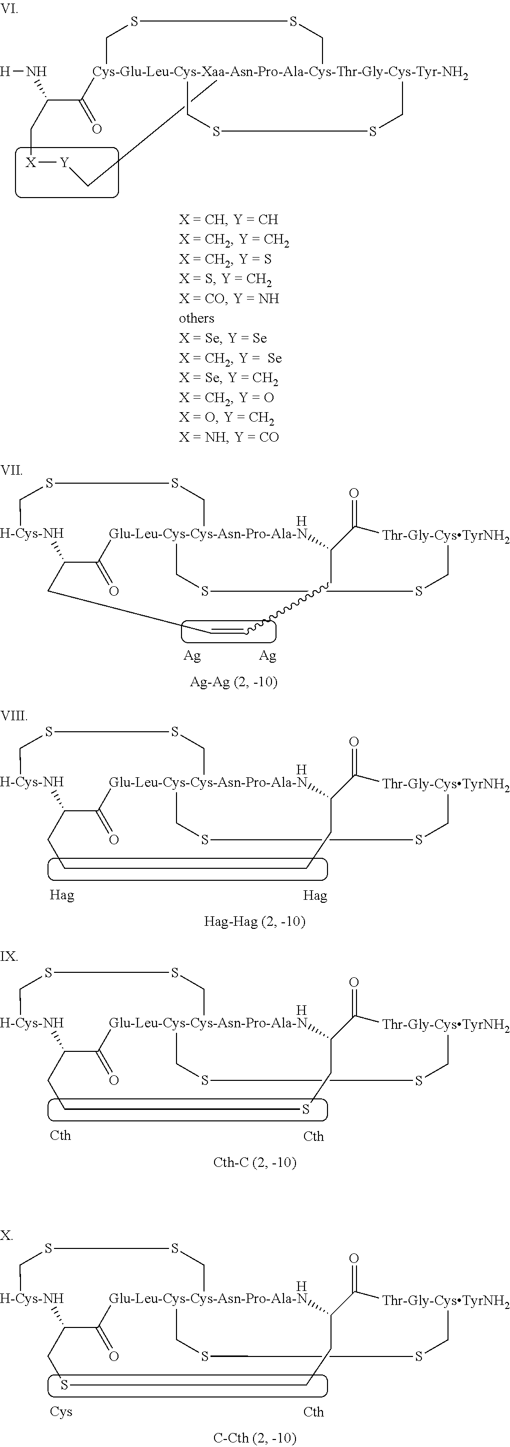

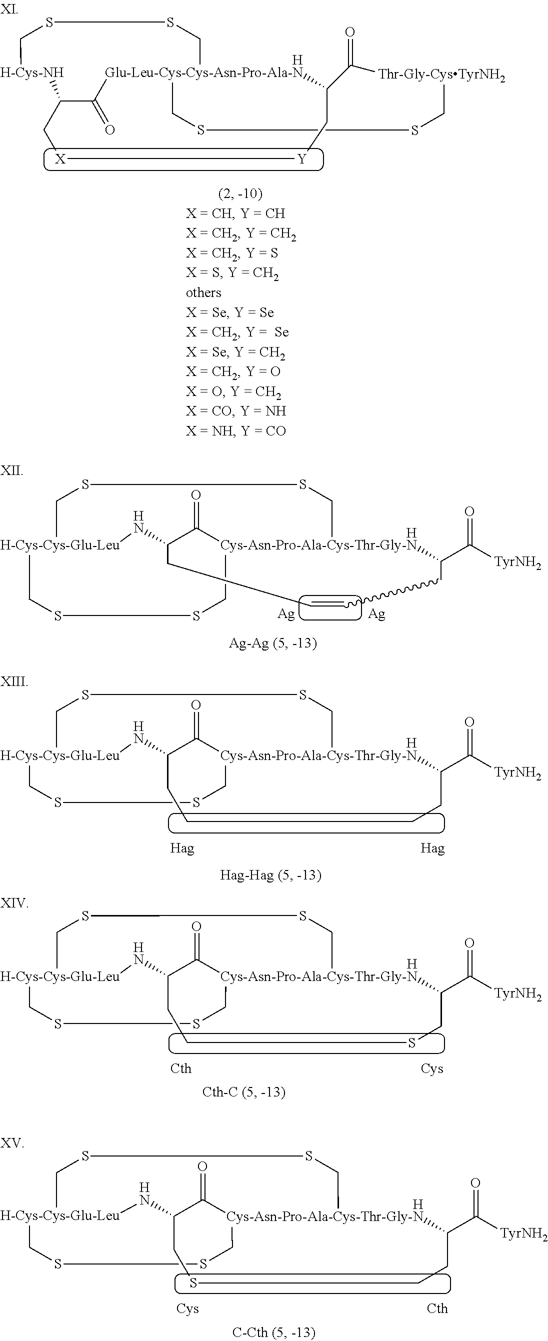

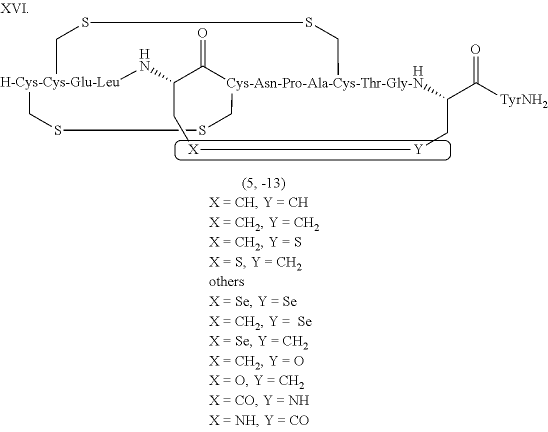

### Peptides

In one aspect, the present invention provides a peptide or a pharmaceutically acceptable salt thereof useful for the methods described herein, wherein the peptide comprises the amino acid sequence:
Xaa₁ Xaa₂ Xaa₃ Xaa₄ Xaa₅ Xaa₆ Xaa₇ Xaa₈ Xaa₉ Xaa₁₀ Xaa₁₁ Xaa₁₂ Xaa₁₃ Xaa₁₄ Ala₁₅ Xaa₁₆ Xaa₁₇ Gly₁₈ Xaa₁₉ Xaa₂₀ Xaa₂₁ (SEQ ID NO: 1), or a pharmaceutically acceptable salt thereof; wherein
Xaa₁ is BE or is absent;
Xaa₂ is BK, Asn or is absent;
Xaa₃ is Asn, Ser or is absent;
Xaa₄ is Ser or is absent;
Xaa₅ is Ser, Asn, Ile, BE or is absent;
Xaa₆ is Tyr, Asp, 4-fluorophenylalanine ((4-F)Phe), BK or is absent;
Xaa₇ is Cys, cystathionine (Cth), allylglycine (Ag), Hag, or Asp;
Xaa₈ is Cys, cystathionine (Cth), penicillamine (Pen), or allylglycine (Ag);
Xaa₉ is Glu, Asp, Ser, Thr, or Gln;
Xaa₁₀ is Leu, cyclohexylalanine (Cha), Phe, or 4-fluorophenylalanine ((4-F)Phe);
Xaa₁₁ is Cys, Ag, or penicillamine (Pen);
Xaa₁₂ is Cys, allylglycine (Ag), Hag, Cth, Dpr, or Val;
Xaa₁₃ is Asn or Leu;
Xaa₁₄ is Pro, Val, sarcosine (Sar), Leu, or Hydroxyproline (OH-Pro);
Xaa₁₆ is Cys, Ag, Pen or Cth;
Xaa₁₇ is Tyr, Thr, cyclohexylalanine (Cha), 4-fluorophenylalanine ((4-F)Phe), Phe, Ser, or Ala;
Xaa₁₉ is Cys, Ag or Pen;
Xaa₂₀ is Tyr, Leu, 4-fluorophenylalanine ((4-F)Phe), cyclohexylalanine (Cha), D-Tyr, N-Methyl Tyr (Nme-Tyr) or is absent;
Xaa₂₁ is absent or Asn;
wherein at least one Xaa is BE, BK, (4-F)Phe, Cth, Ag, Hag, Pen, Cha, Sar, Dpr, or OH-Pro; and
   wherein the peptide contains a covalent bond between Xaa₇ and Xaa₁₂, Xaa₈ and Xaa₁₆, and Xaa₁₁ and Xaa₁₉.

In some embodiments, the N-terminus of the peptide is acetylated or modified at the N-terminus to provide added stability to the peptide. In other embodiments, the N-terminus is capped with pentenoic acid, biotin, 4-Mepip (1-methyl-4-carboxylic acid), C12 alkyl carboxylic acid, C14 alkyl carboxylic acid, C16 alkyl carboxylic acid, or C18 alkyl carboxylic acid. In some embodiments, the C═C double bond of the pentenoic acid may be cyclized with another C═C double bond to form a dicarba bond.

In some embodiments, the C-terminus of the peptide is amidated or modified at the C-terminus to provide added stability to the peptide.

In yet another embodiment, the N-terminus of the peptide is acetylated or capped and the C-terminus is amidated.

In some embodiments, dicarba bonds (CH₂—CH═CH—CH₂) or other covalent bonds described herein between peptide residues may be useful in stabilizing the peptide. The dicarba bond and other covalent bonds described herein may in some instances provide greater stability of the peptide than a disulfide bond. In some embodiments, the dicarba bonds may be reduced (CH₂—CH₂—CH₂—CH₂).

In some embodiments, the enhanced stability of the peptides allows for storage at room temperature for extended periods of time.

In some embodiments, Xaa₇ and Xaa₁₂ are both Ag and a dicarba bond is present between Ag₇ and Ag₁₂; Xaa₇ and Xaa₁₂ are both Cys and a disulfide bond is present between Cys₇ and Cys₁₂; Xaa₈ and Xaa₁₆ are both Cys and a disulfide bond is present between Cys₈ and Cys₁₆; Xaa₁₁ and Xaa₁₉ are both Cys and a disulfide bond is present between Cys₁₁ and Cys₁₉; or any combination thereof.

In other embodiments, Xaa₇ is Cth, Xaa₁₂ is Cys and a bond is present between Cth₇ and Cys₁₂; Xaa₈ is Cth, Xaa₁₆ is Cys and a bond is present between Cth₈ and Cys₁₆; or any combination thereof.

In some embodiments, Xaa₇ is allylglycine or Cys.

In some embodiments, Xaa₈ is Cys or cystathionine.

In some embodiments, Xaa₉ is Glu.

In some embodiments, Xaa₁₀ is Leu.

In some embodiments, Xaa₁₂ is Cys or allylglycine.

In some embodiments, Xaa₁₄ is Val or Pro.

In some embodiments, Xaa₁₇ is Tyr or Thr.

In some embodiments, Xaa₂₀ is Tyr or is absent.

In some embodiments, Xaa₂₁ is absent.

In some embodiments, Xaa₁ is absent; Xaa₂ is absent; Xaa₃ is absent; Xaa₄ is absent; Xaa₅ is absent; Xaa₆ is absent; Xaa₇ is Ag, Cys, or Cth; Xaa₈ is Cys or Cth; Xaa₉ is Glu; Xaa₁₀ is Leu; Xaa₁₂ is Ag or Cys; Xaa₁₄ is Val or Pro; Xaa₁₇ is Tyr or Thr; and Xaa₂₀ is Tyr or is absent.

In some embodiments, Xaa₁₄ is not Pro.

In some embodiments, Xaa₁₇ is not Phe.

In some embodiments, a peptide or pharmaceutically acceptable salt thereof is provided, wherein the peptide comprises the amino acid sequence:

(SEQ ID NO: 2)
H-Ag Cys Glu Leu Cys Ag Asn Pro Ala Cys Thr Gly
Cys Tyr-NH₂;
(SEQ ID NO: 3)
C12-BE BK Asn Ser Ser Tyr Cys Cys Glu Leu Cys
Cys Asn Pro Ala Cys Thr Gly Cys Tyr-NH₂;
(SEQ ID NO: 4)
C16-BE BK Asn Ser Ser Tyr Cys Cys Glu Leu Cys
Cys Asn Pro Ala Cys Thr Gly Cys Tyr-NH₂;
(SEQ ID NO: 5)
C12-BE BK Asn Ser Ser Tyr Cys Cys Glu Leu Cys
Cys Asn Pro Ala Cys Thr Gly Cys Tyr-COOH;
(SEQ ID NO: 6)
C14-BE BK Asn Ser Ser Tyr Cys Cys Glu Leu Cys Cys
Asn Pro Ala Cys Thr Gly Cys Tyr-COOH;
(SEQ ID NO: 7)
C16-BE BK Asn Ser Ser Tyr Cys Cys Glu Leu Cys
Cys Asn Pro Ala Cys Thr Gly Cys Tyr-COOH;
(SEQ ID NO: 8)
H-Cys Ag Glu Leu Cys Cys Asn Pro Ala Ag Thr Gly
Cys Tyr-NH₂;
(SEQ ID NO: 9)
H-Asn Asp Asp Ag Glu Leu Cys Val Asn Val Ala Ag
Thr Gly Cys Leu-NH₂;
(SEQ ID NO: 10)
H-Asn Asp Asp Cys Glu Leu Ag Val Asn Val Ala Cys
Thr Gly Ag Leu-NH₂;
(SEQ ID NO: 11)
C18-BE BK Asn Ser Ser Tyr Cys Cys Glu Leu Cys
Cys Asn Pro Ala Cys Thr Gly Cys Tyr-COOH;
(SEQ ID NO: 12)
H-Ag Cys Glu Leu Cys Ag Asn Pro Ala Cys Thr Gly
Cys-NH₂;
(SEQ ID NO: 13)
H-Ag Cys Glu Leu Cys Ag Asn Pro Ala Cys Ala
Gly Cys-NH₂;
(SEQ ID NO: 14)
H-Ag Cys Glu Phe Cys Ag Asn Pro Ala Cys Thr
Gly Cys Tyr-NH₂;
(SEQ ID NO: 15)
H-Ag Cys Glu (4-F)Phe Cys Ag Asn Pro Ala Cys
Thr Gly Cys-NH₂;
(SEQ ID NO: 16)
H-Ag Cys Glu (4-F)Phe Cys Ag Asn Pro Ala Cys
Thr Gly Cys Tyr-NH₂;
(SEQ ID NO: 17)
H-Ag Cys Glu (4-F)Phe Cys Ag Asn Pro Ala Cys
Thr Gly Cys (4-F)Phe-NH₂;
(SEQ ID NO: 18)
H-Cys Cys Glu Leu Ag Cys Asn Pro Ala Cys Thr
Gly Ag Tyr-NH₂;
(SEQ ID NO: 19)
H-Ag Cys Glu Phe Cys Ag Asn Pro Ala Cys Thr
Gly Cys-NH₂;
(SEQ ID NO: 20)
H-Ag Cys Glu Phe Cys Ag Asn Pro Ala Cys Thr
Gly Cys (4-F)Phe-NH₂;
(SEQ ID NO: 21)
H-Cys Cys Glu (4-F)Phe Cys Cys Asn Pro Ala Cys
Thr Gly Cys Tyr-NH₂;
(SEQ ID NO: 22)
H-Cys Cys Glu (4-F)Phe Cys Cys Asn Pro Ala Cys
Thr Gly Cys (4-F)Phe-NH₂;
(SEQ ID NO: 23)
H-Ag Cys Glu Leu Cys Ag Asn Pro Ala Cys Thr Gly
Cys (4-F)Phe-NH₂;
(SEQ ID NO: 24)
H-(4-F)Phe Ag Cys Glu Leu Cys Ag Asn Pro Ala
Cys Thr Gly Cys (4-F)Phe-NH₂;
(SEQ ID NO: 25)
H-Ag Cys Glu Leu Cys Ag Asn Pro Ala Cys Ala Gly
Cys Tyr-NH₂;
(SEQ ID NO: 26)
H-Ag Cys Glu Leu Cys Ag Asn Pro Ala Cys Tyr
Gly Cys Tyr-NH₂;
(SEQ ID NO: 27)
H-Ag Cys Glu Leu Cys Ag Asn Pro Ala Cys Phe
Gly Cys Tyr-NH₂;
(SEQ ID NO: 28)
H-Ag Cys Glu Leu Cys Ag Asn Pro Ala Cys (4-F)Phe
Gly Cys Tyr-NH₂;
(SEQ ID NO: 29)
H-Ag Cys Glu Leu Cys Ag Asn Pro Ala Cys Thr
Gly Cys Leu-NH₂;
(SEQ ID NO: 30)
H-Ag Cys Glu Leu Cys Ag Asn Pro Ala Cys Thr
Gly Cys Cha-NH₂;
(SEQ ID NO: 31)
H-Ag Cys Glu Cha Cys Ag Asn Pro Ala Cys Thr
Gly Cys Tyr-NH₂;
(SEQ ID NO: 32)
H-Ag Cys Glu Leu Cys Ag Asn Pro Ala Cys Thr
Gly Cys D-Tyr-NH₂;
(SEQ ID NO: 33)
H-Ag Pen Glu Leu Cys Ag Asn Pro Ala Pen Thr
Gly Cys Tyr-NH₂;
(SEQ ID NO: 34)
H-Ag Cys Glu Leu Cys Ag Asn Pro Ala Cys Thr
Gly Cys Leu Asn-NH₂;
(SEQ ID NO: 35)
H-Ag Cys Glu Leu Cys Ag Asn Pro Ala Cys Thr Gly
Cys Tyr Asn-NH₂;
(SEQ ID NO: 36)
H-Asp Ag Cys Glu Leu Cys Ag Asn Pro Ala Cys
Thr Gly Cys Tyr-NH₂;
(SEQ ID NO: 37)
H-Ile Asp Ag Cys Glu Leu Cys Ag Asn Pro Ala
Cys Thr Gly Cys Tyr-NH₂;
(SEQ ID NO: 38)
H-Ag Cys Glu Leu Cys Ag Asn OH-Pro Ala Cys
Thr Gly Cys Tyr-NH₂;
(SEQ ID NO: 39)
H-Ag Cys Glu Leu Cys Ag Asn OH-Pro Ala Cys
Thr Gly Cys-NH₂;
(SEQ ID NO: 40)
H-Ag Cys Glu Leu Pen Ag Asn Pro Ala Cys Thr
Gly Pen Tyr-NH₂;
(SEQ ID NO: 41)
H-Ag Cys Glu Leu Pen Ag Asn Pro Ala Cys Thr
Gly Pen Tyr-NH₂;
(SEQ ID NO: 42)
H-Ag Cys Glu Leu Cys Ag Asn Pro Ala Cys Tyr Gly
Cys Nme-Tyr-NH₂;
(SEQ ID NO: 43)
H-Ag Cys Glu Leu Cys Ag Asn Pro Ala Cys Tyr Gly
Cys-COOH;
(SEQ ID NO: 44)
H-Ag Cys Glu Leu Cys Ag Asn Pro Ala Cys Cha
Gly Cys Tyr-NH₂;
(SEQ ID NO: 45)
H-Ag Cys Glu Leu Cys Ag Asn Leu Ala Cys Thr
Gly Cys Tyr-NH₂;
(SEQ ID NO: 46)
H-Ag Cys Glu Leu Cys Ag Asn Sar Ala Cys Thr
Gly Cys Tyr-NH₂;
(SEQ ID NO: 47)
H-Ag Cys Glu Leu Cys Ag Asn Val Ala Cys Thr
Gly Cys Tyr-NH₂;
(SEQ ID NO: 48)
H-Ag Cys Glu Leu Cys Ag Asn Pro Ala Cys Tyr
Gly Cys Nme-Tyr-NH₂;
(SEQ ID NO: 49)
H-Ag Cys Glu Leu Cys Ag Leu Pro Ala Cys Tyr
Gly Cys-COOH;
(SEQ ID NO: 50)
H-Ag Cys Asp Leu Cys Ag Asn Pro Ala Cys Tyr
Gly Cys-COOH;
(SEQ ID NO: 51)
H-Ag Cys Ser Leu Cys Ag Asn Pro Ala Cys Tyr
Gly Cys-COOH;
(SEQ ID NO: 52)
H-Ag Cys Thr Leu Cys Ag Asn Pro Ala Cys Tyr
Gly Cys-COOH;
(SEQ ID NO: 53)
H-Hag Cys Glu Leu Cys Hag Asn Pro Ala Cys
Thr Gly Cys Tyr-NH₂;
(SEQ ID NO: 54)
H-Ag Cys Gln Leu Cys Ag Asn Pro Ala Cys Tyr
Gly Cys-COOH;
(SEQ ID NO: 55)
H-Ag Cys Glu Leu Cys Ag Leu Pro Ala Cys Thr
Gly Cys Tyr-NH₂;
(SEQ ID NO: 56)
Pent-Cys Glu Leu Cys Ag Asn Pro Ala Cys Tyr
Gly Cys Tyr-NH₂;
(SEQ ID NO: 57)
H-Cys Cys Glu Leu Cys Ag Asn Pro Ala Cys
Tyr Gly Ag-COOH;
(SEQ ID NO: 58)
Pent-Cys Glu Leu Cys Ag Asn Pro Ala Cys Tyr
Gly Cys-COOH;
(SEQ ID NO: 59)
H-Hag Cys Glu Leu Cys Hag Asn Pro Ala Cys
Tyr Gly Cys-COOH;
(SEQ ID NO: 60)
H-Ag Cys Glu Leu Cys Ag Asn Val Ala Cys Tyr
Gly Cys Tyr-NH₂;
(SEQ ID NO: 61)
H-Ag Cys Glu Leu Cys Ag Asn Val Ala Cys Tyr
Gly Cys-COOH;
(SEQ ID NO: 62)
H-Cys Cth Glu Leu Cys Cys Asn Pro Ala Cys
Tyr Gly Cys Tyr-NH₂;
(SEQ ID NO: 63)
H-Cth Cys Glu Leu Cys Cys Asn Pro Ala Cys
Tyr Gly Cys Tyr-NH₂;
(SEQ ID NO: 64)
H-Cth Cys Glu Leu Cys Cys Asn Pro Ala Cys
Tyr Gly Cys-COOH;
(SEQ ID NO: 65)
H-Cys Cth Glu Leu Cys Cys Asn Val Ala Cys Tyr
Gly Cys Tyr-NH₂;
(SEQ ID NO: 66)
H-Cys Cth Glu Leu Cys Cys Asn Val Ala Cys
Thr Gly Cys Tyr-NH₂;
(SEQ ID NO: 67)
Ac-Cys Cth Glu Leu Cys Cys Asn Val Ala Cys
Thr Gly Cys Tyr-NH₂;
(SEQ ID NO: 68)
Ac-Cys Cth Glu Leu Cys Cys Asn Pro Ala Cys
Tyr Gly Cys Tyr-NH₂;
(SEQ ID NO: 69)
Ac-Cys Cth Glu Leu Cys Cys Asn Val Ala Cys
Tyr Gly Cys-COOH;
(SEQ ID NO: 70)
H-Cys Cth Glu Leu Cys Cys Asn Val Ala Cys
Tyr Gly Cys-COOH;
(SEQ ID NO: 71)
Ac-Cys Cth Glu Leu Cys Cys Asn Pro Ala Cys
Tyr Gly Cys-COOH;
(SEQ ID NO: 72)
H-Cys Cys Glu Leu Cys Asn Val Ala Cth Tyr
Gly Cys-COOH;
(SEQ ID NO: 73)
H-Cys Cys Glu Leu Cys Cys Asn Val Ala Cth Tyr
Gly Cys Tyr-NH₂;
(SEQ ID NO: 74)
H-Cys Cys Glu Leu Cys Cys Asn Val Ala Cth Thr
Gly Cys Tyr-NH₂;
(SEQ ID NO: 75)
H-Cys Cys Glu Leu Cys Cth Asn Val Ala Cys
Tyr Gly Cys-COOH;
(SEQ ID NO: 76)
H-Cys Cys Glu Leu Cys Cth Asn Val Ala Cys
Tyr Gly Cys Tyr-NH₂;
(SEQ ID NO: 77)
H-Cys Cys Glu Leu Cys Cth Asn Val Ala Cys
Thr Gly Cys Tyr-NH₂;
(SEQ ID NO: 78)
4-Mepip-Cys Cth Glu Leu Cys Cys Asn Val Ala
Cys Tyr Gly Cys-COOH;
(SEQ ID NO: 79)
H-Cys Cth Glu Leu Cys Cys Asn Val Ala Cys
Thr Gly Cys-COOH;
(SEQ ID NO: 80)
H-Cys Cth Glu Leu Cys Cys Asn Val Ala Cys
Thr Gly Cys-NH₂;
(SEQ ID NO: 81)
H-Cys Cth Glu Leu Cys Cys Asn Val Ala Cys
Phe Gly Cys-COOH;
(SEQ ID NO: 82)
H-Ag Ag Glu Leu Ag Ag Asn Pro Ala Ag Thr
Gly Ag Tyr-COOH;
(SEQ ID NO: 83)
H-Cys Cth Glu Leu Cys Cys Asn Ala Ala Cys
Thr Gly Cys Tyr-NH₂;
(SEQ ID NO: 84)
H-Asp Cys Glu Leu Cys Dpr Asn Val Ala Cys
Thr Gly Cys Tyr-NH₂;
(SEQ ID NO: 85)
H-Cys Cth Glu Leu Cys Cys Asn Val Ala Cys
Ser Gly Cys Tyr-NH₂;
(SEQ ID NO: 86)
H-Cth Glu Leu Cys Ag Asn Val Ala Cys Thr Gly
Cys Tyr-NH₂;
(SEQ ID NO: 87)
H-Ag Cys Glu Leu Cys Ag Asn Val Ala Cys Thr
Gly Cys Tyr-NH₂;
(SEQ ID NO: 88)
H-Ag Cys Glu Leu Cys Ag Asn Pro Ala Cys
Thr Gly Cys Tyr-NH₂;
(SEQ ID NO: 89)
H-Cth Cys Glu Leu Cys Cys Asn Val Ala Cys
Thr Gly Cys-COOH;
(SEQ ID NO: 90)
H-Cth Cys Glu Leu Cys Cys Asn Val Ala Cys
Thr Gly Cys Tyr-NH₂;
(SEQ ID NO: 91)
H-Cth Cys Glu Leu Cys Cys Asn Val Ala Cys
Tyr Gly Cys Tyr-NH₂;
or
(SEQ ID NO: 92)
H-Cys Cys Glu Leu Cys Cys Asn Val Ala Cth
Tyr Gly Cys-COOH,

terminus capped with 4-Mepip (1-methyl-piperidine-4-carboxylic acid), C12- indicates an N-terminus capped with C12 alkyl carboxylic acid, C14- indicates an N-terminus capped with C14 alkyl carboxylic acid, C16- indicates an N-terminus capped with C16 alkyl carboxylic acid, C18- indicates an N-terminus capped with C18 alkyl carboxylic acid, H- indicates an unmodified N-terminus, —NH₂ indicates an amidated C-terminus and —COOH indicates an unmodified C-terminus.

In further embodiments, dicarba bond between two Ag residues can be either the cis or trans isomer at the dicarba bond. As used herein, a cis isomer of a dicarba bond has both hydrogen atoms on the same side of the C═C double bond, and a trans isomer of a dicarba bond has hydrogen atoms on opposite sides of the C═C double bond.

In some embodiments, a peptide or pharmaceutically acceptable salt thereof is provided, wherein the peptide comprises peptide comprises no more than 50, 40, 30 or 20 amino acids. In further embodiments, the peptide comprises no more than 19, 18, 17, 16, 15 or 14 amino acids.

In another aspect, the present invention provides a peptide or a pharmaceutically acceptable salt thereof, wherein the peptide consists of the amino acid sequence
Xaa₁ Xaa₂ Xaa₃ Xaa₄ Xaa₅ Xaa₆ Xaa₇ Xaa₈ Xaa₉ Xaa₁₀ Xaa₁₁ Xaa₁₂ Xaa₁₃ Xaa₁₄ Ala₁₅ Xaa₁₆ Xaa₁₇ Gly₁₈ Xaa₁₉ Xaa₂₀ Xaa₂₁ (SEQ ID NO: 1), or a pharmaceutically acceptable salt thereof; wherein
Xaa₁ is BE or is absent;
Xaa₂ is BK, Asn or is absent;
Xaa₃ is Asn, Ser or is absent;
Xaa₄ is Ser or is absent;
Xaa₅ is Ser, Asn, Ile, BE or is absent;
Xaa₆ is Tyr, Asp, 4-fluorophenylalanine ((4-F)Phe), BK or is absent;
Xaa₇ is Cys, cystathionine (Cth), allylglycine (Ag), Hag, or Asp;
Xaa₈ is Cys, cystathionine (Cth), penicillamine (Pen), or allylglycine (Ag);
Xaa₉ is Glu, Asp, Ser, Thr, or Gln;
Xaa₁₀ is Leu, cyclohexylalanine (Cha), Phe, or 4-fluorophenylalanine ((4-F)Phe);
Xaa₁₁ is Cys, Ag, or penicillamine (Pen);
Xaa₁₂ is Cys, allylglycine (Ag), Hag, Cth, Dpr, or Val;
Xaa₁₃ is Asn or Leu;
Xaa₁₄ is Pro, Val, sarcosine (Sar), Leu, or Hydroxyproline (OH-Pro);
Xaa₁₆ is Cys, Ag, Pen or Cth;
Xaa₁₇ is Tyr, Thr, cyclohexylalanine (Cha), 4-fluorophenylalanine ((4-F)Phe), Phe, Ser, or Ala;
Xaa₁₉ is Cys, Ag or Pen;
Xaa₂₀ is Tyr, Leu, 4-fluorophenylalanine ((4-F)Phe), cyclohexylalanine (Cha), D-Tyr, N-Methyl Tyr (Nme-Tyr) or is absent;
Xaa₂₁ is absent or Asn;
wherein at least one Xaa is BE, BK, (4-F)Phe, Cth, Ag, Hag, Pen, Cha, Sar, Dpr, or OH-Pro
and wherein the peptide contains a covalent bond between Xaa₇ and Xaa₁₂, Xaa₈ and Xaa₁₆ and Xaa₁₁ and Xaa₁₉.

In some embodiments, the N-terminus of the peptide is acetylated to provide added stability to the peptide. In other embodiments, the N-terminus is capped with pentenoic acid, biotin, 4-Mepip (1-methyl-piperidine-4-carboxylic acid), C12 alkyl carboxylic acid, C14 alkyl carboxylic acid, C16 alkyl carboxylic acid, or C18 alkyl carboxylic acid. In some embodiments, the C═C double bond of the pentenoic acid may be cyclized with another C═C double bond to form a dicarba bond.

In some embodiments, the C-terminus of the peptide is amidated to provide added stability to the peptide.

In some embodiments, Xaa₇ and Xaa₁₂ are both Ag and a dicarba bond is present between Ag₇ and Ag₁₂; Xaa₇ and Xaa₁₂ are both Cys and a disulfide bond is present between Cys₇ and Cys₁₂; Xaa₈ and Xaa₁₆ are both Cys and a disulfide bond is present between Cys₈ and Cys₁₆; Xaa₁₁ and Xaa₁₉ are both Cys, and a disulfide bond is present between Cys₁₁ and Cys₁₉; or any combination thereof.

In some embodiments, Xaa₇ is allylglycine or Cys.

In some embodiments, Xaa₈ is Cys or cystathionine.

In some embodiments, Xaa₉ is Glu.

In some embodiments, Xaa₁₀ is Leu.

In some embodiments, Xaa₁₂ is Cys or allylglycine.

In some embodiments, Xaa₁₄ is Val or Pro.

In some embodiments, Xaa₁₇ is Tyr or Thr.

In some embodiments, Xaa₂₀ is Tyr or is absent.

In some embodiments, Xaa₂₁ is absent.

In some embodiments, Xaa₁ is absent; Xaa₂ is absent; Xaa₃ is absent; Xaa₄ is absent; Xaa₅ is absent; Xaa₆ is absent; Xaa₇ is Ag, Cys, or Cth; Xaa₈ is Cys or Cth; Xaa₉ is Glu; Xaa₁₀ is Leu; Xaa₁₂ is Ag or Cys; Xaa₁₄ is Val or Pro; Xaa₁₇ is Tyr or Thr; and Xaa₂₀ is Tyr or is absent.

In some embodiments, a peptide or pharmaceutically acceptable salt thereof is provided, wherein the peptide consists of the amino acid sequence:

(SEQ ID NO: 2)
H-Ag Cys Glu Leu Cys Ag Asn Pro Ala Cys Thr
Gly Cys Tyr-NH₂;
(SEQ ID NO: 3)
C12-BE BK Asn Ser Ser Tyr Cys Cys Glu Leu Cys
Cys Asn Pro Ala Cys Thr Gly Cys Tyr-NH₂;
(SEQ ID NO: 4)
C16-BE BK Asn Ser Ser Tyr Cys Cys Glu Leu Cys
Cys Asn Pro Ala Cys Thr Gly Cys Tyr-NH₂;
(SEQ ID NO: 5)
C12-BE BK Asn Ser Ser Tyr Cys Cys Glu Leu Cys
Cys Asn Pro Ala Cys Thr Gly Cys Tyr-COOH;
(SEQ ID NO: 6)
C14-BE BK Asn Ser Ser Tyr Cys Cys Glu Leu Cys
Cys Asn Pro Ala Cys Thr Gly Cys Tyr-COOH;
(SEQ ID NO: 7)
C16-BE BK Asn Ser Ser Tyr Cys Cys Glu Leu Cys
Cys Asn Pro Ala Cys Thr Gly Cys Tyr-COOH;
(SEQ ID NO: 8)
H-Cys Ag Glu Leu Cys Cys Asn Pro Ala Ag Thr Gly
Cys Tyr-NH₂;
(SEQ ID NO: 9)
H-Asn Asp Asp Ag Glu Leu Cys Val Asn Val Ala Ag
Thr Gly Cys Leu-NH₂;
(SEQ ID NO: 10)
H-Asn Asp Asp Cys Glu Leu Ag Val Asn Val Ala
Cys Thr Gly Ag Leu-NH₂;
(SEQ ID NO: 11)
C18-BE BK Asn Ser Ser Tyr Cys Cys Glu Leu Cys
Cys Asn Pro Ala Cys Thr Gly Cys Tyr-COOH;
(SEQ ID NO: 12)
H-Ag Cys Glu Leu Cys Ag Asn Pro Ala Cys Thr Gly
Cys-NH₂;
(SEQ ID NO: 13)
H-Ag Cys Glu Leu Cys Ag Asn Pro Ala Cys Ala Gly
Cys-NH₂;
(SEQ ID NO: 14)
H-Ag Cys Glu Phe Cys Ag Asn Pro Ala Cys Thr Gly
Cys Tyr-NH₂;
(SEQ ID NO: 15)
H-Ag Cys Glu (4-F)Phe Cys Ag Asn Pro Ala Cys Thr
Gly Cys-NH₂;
(SEQ ID NO: 16)
H-Ag Cys Glu (4-F)Phe Cys Ag Asn Pro Ala Cys Thr
Gly Cys Tyr-NH₂;
(SEQ ID NO: 17)
H-Ag Cys Glu (4-F)Phe Cys Ag Asn Pro Ala Cys Thr
Gly Cys (4-F)Phe-NH₂;
(SEQ ID NO: 18)
H-Cys Cys Glu Leu Ag Cys Asn Pro Ala Cys Thr Gly
Ag Tyr-NH₂;
(SEQ ID NO: 19)
H-Ag Cys Glu Phe Cys Ag Asn Pro Ala Cys Thr Gly
Cys-NH₂;
(SEQ ID NO: 20)
H-Ag Cys Glu Phe Cys Ag Asn Pro Ala Cys Thr Gly
Cys (4-F)Phe-NH₂;
(SEQ ID NO: 21)
H-Cys Cys Glu (4-F)Phe Cys Cys Asn Pro Ala Cys
Thr Gly Cys Tyr-NH₂;
(SEQ ID NO: 22)
H-Cys Cys Glu (4-F)Phe Cys Cys Asn Pro Ala Cys
Thr Gly Cys (4-F)Phe-NH₂;
(SEQ ID NO: 23)
H-Ag Cys Glu Leu Cys Ag Asn Pro Ala Cys Thr Gly
Cys (4-F)Phe-NH₂;
(SEQ ID NO: 24)
H-(4-F)Phe Ag Cys Glu Leu Cys Ag Asn Pro Ala
Cys Thr Gly Cys (4-F)Phe-NH₂;
(SEQ ID NO: 25)
H-Ag Cys Glu Leu Cys Ag Asn Pro Ala Cys Ala
Gly Cys Tyr-NH₂;
(SEQ ID NO: 26)
H-Ag Cys Glu Leu Cys Ag Asn Pro Ala Cys Tyr
Gly Cys Tyr-NH₂;
(SEQ ID NO: 27)
H-Ag Cys Glu Leu Cys Ag Asn Pro Ala Cys Phe
Gly Cys Tyr-NH₂;
(SEQ ID NO: 28)
H-Ag Cys Glu Leu Cys Ag Asn Pro Ala Cys (4-F)Phe
Gly Cys Tyr-NH₂;
(SEQ ID NO: 29)
H-Ag Cys Glu Leu Cys Ag Asn Pro Ala Cys Thr Gly
Cys Leu-NH₂;
(SEQ ID NO: 30)
H-Ag Cys Glu Leu Cys Ag Asn Pro Ala Cys Thr Gly
Cys Cha-NH₂;
(SEQ ID NO: 31)
H-Ag Cys Glu Cha Cys Ag Asn Pro Ala Cys Thr Gly
Cys Tyr-NH₂;
(SEQ ID NO: 32)
H-Ag Cys Glu Leu Cys Ag Asn Pro Ala Cys Thr Gly
Cys D-Tyr-NH₂;
(SEQ ID NO: 33)
H-Ag Pen Glu Leu Cys Ag Asn Pro Ala Pen Thr Gly
Cys Tyr-NH₂;
(SEQ ID NO: 34)
H-Ag Cys Glu Leu Cys Ag Asn Pro Ala Cys Thr Gly
Cys Leu Asn-NH₂;
(SEQ ID NO: 35)
H-Ag Cys Glu Leu Cys Ag Asn Pro Ala Cys Thr Gly
Cys Tyr Asn-NH₂;
(SEQ ID NO: 36)
H-Asp Ag Cys Glu Leu Cys Ag Asn Pro Ala Cys Thr
Gly Cys Tyr-NH₂;
(SEQ ID NO: 37)
H-Ile Asp Ag Cys Glu Leu Cys Ag Asn Pro Ala Cys
Thr Gly Cys Tyr-NH₂;
(SEQ ID NO: 38)
H-Ag Cys Glu Leu Cys Ag Asn OH-Pro Ala Cys Thr
Gly Cys Tyr-NH₂;
(SEQ ID NO: 39)
H-Ag Cys Glu Leu Cys Ag Asn OH-Pro Ala Cys Thr
Gly Cys-NH₂;
(SEQ ID NO: 40)
H-Ag Cys Glu Leu Pen Ag Asn Pro Ala Cys Thr Gly
Pen Tyr-NH₂;
(SEQ ID NO: 41)
H-Ag Cys Glu Leu Pen Ag Asn Pro Ala Cys Thr Gly
Pen Tyr-NH₂;
(SEQ ID NO: 42)
H-Ag Cys Glu Leu Cys Ag Asn Pro Ala Cys Tyr Gly
Cys Nme-Tyr-NH₂;
(SEQ ID NO: 43)
H-Ag Cys Glu Leu Cys Ag Asn Pro Ala Cys Tyr Gly
Cys-COOH;
(SEQ ID NO: 44)
H-Ag Cys Glu Leu Cys Ag Asn Pro Ala Cys Cha Gly
Cys Tyr-NH₂;
(SEQ ID NO: 45)
H-Ag Cys Glu Leu Cys Ag Asn Leu Ala Cys Thr Gly
Cys Tyr-NH₂;
(SEQ ID NO: 46)
H-Ag Cys Glu Leu Cys Ag Asn Sar Ala Cys Thr Gly
Cys Tyr-NH₂;
(SEQ ID NO: 47)
H-Ag Cys Glu Leu Cys Ag Asn Val Ala Cys Thr Gly
Cys Tyr-NH₂;
(SEQ ID NO: 48)
H-Ag Cys Glu Leu Cys Ag Asn Pro Ala Cys Tyr Gly
Cys Nme-Tyr-NH₂;
(SEQ ID NO: 49)
H-Ag Cys Glu Leu Cys Ag Leu Pro Ala Cys Tyr Gly
Cys-COOH;
(SEQ ID NO: 50)
H-Ag Cys Asp Leu Cys Ag Asn Pro Ala Cys Tyr Gly
Cys-COOH;
(SEQ ID NO: 51)
H-Ag Cys Ser Leu Cys Ag Asn Pro Ala Cys Tyr Gly
Cys-COOH;
(SEQ ID NO: 52)
H-Ag Cys Thr Leu Cys Ag Asn Pro Ala Cys Tyr Gly
Cys-COOH;
(SEQ ID NO: 53)
H-Hag Cys Glu Leu Cys Hag Asn Pro Ala Cys Thr Gly
Cys Tyr-NH₂;
(SEQ ID NO: 54)
H-Ag Cys Gln Leu Cys Ag Asn Pro Ala Cys Tyr Gly
Cys-COOH;
(SEQ ID NO: 55)
H-Ag Cys Glu Leu Cys Ag Leu Pro Ala Cys Thr Gly
Cys Tyr-NH₂;
(SEQ ID NO: 56)
Pent-Cys Glu Leu Cys Ag Asn Pro Ala Cys Tyr Gly
Cys Tyr-NH₂;
(SEQ ID NO: 57)
H-Cys Cys Glu Leu Cys Ag Asn Pro Ala Cys Tyr
Gly Ag-COOH;
(SEQ ID NO: 58)
Pent-Cys Glu Leu Cys Ag Asn Pro Ala Cys Tyr Gly
Cys-COOH;
(SEQ ID NO: 59)
H-Hag Cys Glu Leu Cys Hag Asn Pro Ala Cys Tyr Gly
Cys-COOH;
(SEQ ID NO: 60)
H-Ag Cys Glu Leu Cys Ag Asn Val Ala Cys Tyr Gly
Cys Tyr-NH₂;
(SEQ ID NO: 61)
H-Ag Cys Glu Leu Cys Ag Asn Val Ala Cys Tyr Gly
Cys-COOH;
(SEQ ID NO: 62)
H-Cys Cth Glu Leu Cys Cys Asn Pro Ala Cys Tyr Gly
Cys Tyr-NH₂;
(SEQ ID NO: 63)
H-Cth Cys Glu Leu Cys Cys Asn Pro Ala Cys Tyr Gly
Cys Tyr-NH₂;
(SEQ ID NO: 64)
H-Cth Cys Glu Leu Cys Cys Asn Pro Ala Cys Tyr Gly
Cys-COOH;
(SEQ ID NO: 65)
H-Cys Cth Glu Leu Cys Cys Asn Val Ala Cys Tyr Gly
Cys Tyr-NH₂;
(SEQ ID NO: 66)
H-Cys Cth Glu Leu Cys Cys Asn Val Ala Cys Thr Gly
Cys Tyr-NH₂;
(SEQ ID NO: 67)
Ac-Cys Cth Glu Leu Cys Cys Asn Val Ala Cys Thr Gly
Cys Tyr-NH₂;
(SEQ ID NO: 68)
Ac-Cys Cth Glu Leu Cys Cys Asn Pro Ala Cys Tyr Gly
Cys Tyr-NH₂;
(SEQ ID NO: 69)
Ac-Cys Cth Glu Leu Cys Cys Asn Val Ala Cys Tyr Gly
Cys-COOH;
(SEQ ID NO: 70)
H-Cys Cth Glu Leu Cys Cys Asn Val Ala Cys Tyr Gly
Cys-COOH;
(SEQ ID NO: 71)
Ac-Cys Cth Glu Leu Cys Cys Asn Pro Ala Cys Tyr Gly
Cys-COOH;
(SEQ ID NO: 72)
H-Cys Cys Glu Leu Cys Asn Val Ala Cth Tyr Gly
Cys-COOH;
(SEQ ID NO: 73)
H-Cys Cys Glu Leu Cys Cys Asn Val Ala Cth Tyr Gly
Cys Tyr-NH₂;
(SEQ ID NO: 74)
H-Cys Cys Glu Leu Cys Cys Asn Val Ala Cth Thr Gly
Cys Tyr-NH₂;
(SEQ ID NO: 75)
H-Cys Cys Glu Leu Cys Cth Asn Val Ala Cys Tyr Gly
Cys-COOH;
(SEQ ID NO: 76)
H-Cys Cys Glu Leu Cys Cth Asn Val Ala Cys Tyr Gly
Cys Tyr-NH₂;
(SEQ ID NO: 77)
H-Cys Cys Glu Leu Cys Cth Asn Val Ala Cys Thr Gly
Cys Tyr-NH₂;
(SEQ ID NO: 78)
4-Mepip-Cys Cth Glu Leu Cys Cys Asn Val Ala Cys
Tyr Gly Cys-COOH;
(SEQ ID NO: 79)
H-Cys Cth Glu Leu Cys Cys Asn Val Ala Cys Thr Gly
Cys-COOH;
(SEQ ID NO: 80)
H-Cys Cth Glu Leu Cys Cys Asn Val Ala Cys Thr Gly
Cys-NH₂;
(SEQ ID NO: 81)
H-Cys Cth Glu Leu Cys Cys Asn Val Ala Cys Phe
Gly Cys-COOH;
(SEQ ID NO: 82)
H-Ag Ag Glu Leu Ag Ag Asn Pro Ala Ag Thr Gly Ag
Tyr-COOH;
(SEQ ID NO: 83)
H-Cys Cth Glu Leu Cys Cys Asn Ala Ala Cys Thr Gly
Cys Tyr-NH₂;
(SEQ ID NO: 84)
H-Asp Cys Glu Leu Cys Dpr Asn Val Ala Cys Thr Gly
Cys Tyr-NH₂;
(SEQ ID NO: 85)
H-Cys Cth Glu Leu Cys Cys Asn Val Ala Cys Ser Gly
Cys Tyr-NH₂;
(SEQ ID NO: 86)
H-Cth Glu Leu Cys Ag Asn Val Ala Cys Thr Gly Cys
Tyr-NH₂;
(SEQ ID NO: 87)
H-Ag Cys Glu Leu Cys Ag Asn Val Ala Cys Thr Gly
Cys Tyr-NH₂;
(SEQ ID NO: 88)
H-Ag Cys Glu Leu Cys Ag Asn Pro Ala Cys Thr Gly
Cys Tyr-NH₂;
(SEQ ID NO: 89)
H-Cth Cys Glu Leu Cys Cys Asn Val Ala Cys Thr
Gly Cys-COOH;
(SEQ ID NO: 90)
H-Cth Cys Glu Leu Cys Cys Asn Val Ala Cys Thr Gly
Cys Tyr-NH₂;
(SEQ ID NO: 91)
H-Cth Cys Glu Leu Cys Cys Asn Val Ala Cys Tyr Gly
Cys Tyr-NH₂;
or
(SEQ ID NO: 92)
H-Cys Cys Glu Leu Cys Cys Asn Val Ala Cth Tyr
Gly Cys-COOH.

In another embodiment, the present invention provides a peptide or a pharmaceutically acceptable salt thereof useful for the methods described herein, wherein the peptide comprises or consists of the amino acid sequence:
Xaa₁ Xaa₂ Xaa₃ Xaa₄ Xaa₅ Xaa₆ Xaa₇ Xaa₈ Xaa₉ Xaa₁₀ Cys₁₁ Xaa₁₂ Asn₁₃ Xaa₁₄ Ala₁₅ Cys₁₆ Xaa₁₇ Gly₁₈ Cys₁₉ Xaa₂₀ Xaa₂₁ (SEQ ID NO: 93), or a pharmaceutically acceptable salt thereof; wherein
Xaa₁ is BE or is absent;
Xaa₂ is BK or is absent;
Xaa₃ is Asn or is absent;
Xaa₄ is Ser or is absent;
Xaa₅ is Ser, Asn, Ile, or is absent;
Xaa₆ is Tyr, Asp, 4-fluorophenylalanine ((4-F)Phe), or is absent;
Xaa₇ is Cys, cystathionine (Cth), allylglycine (Ag), Hag, or Asp;
Xaa₈ is Cys, cystathionine (Cth), penicillamine (Pen), or allylglycine (Ag);
Xaa₉ is Glu, Asp, Ser, Thr, or Gln;
Xaa₁₀ is Leu, cyclohexylalanine (Cha), Phe, or 4-fluorophenylalanine ((4-F)Phe);
Xaa₁₂ is Cys, allylglycine (Ag), Hag, or Val;
Xaa₁₄ is Pro, Val, sarcosine (Sar), Leu, or Hydroxyproline (OH-Pro);
Xaa₁₇ is Tyr, Thr, cyclohexylalanine (Cha), 4-fluorophenylalanine ((4-F)Phe), Phe, or Ala;
Xaa₂₀ is Tyr, Leu, 4-fluorophenylalanine ((4-F)Phe), cyclohexylalanine (Cha), D-Tyr, N-Methyl Tyr (Nme-Tyr) or is absent;
Xaa₂₁ is absent or Asn;
wherein at least one Xaa is BE, BK, (4-F)Phe, Cth, Ag, Hag, Pen, Cha, Sar, or OH-Pro; and wherein the peptide contains a covalent bond between Xaa₇ and Xaa₁₂, Xaa₈ and Cys₁₆ and Cys₁₁ and Cys₁₉.

In further embodiments, the peptide or a pharmaceutically acceptable salt thereof, wherein the peptide comprises or consists of the amino acid sequence:
Xaa₁ Xaa₂ Xaa₃ Xaa₄ Xaa₅ Xaa₆ Xaa₇ Xaa₈ Xaa₉ Xaa₁₀ Cys₁₁ Xaa₁₂ Asn₁₃ Xaa₁₄ Ala₁₅ Cys₁₆ Xaa₁₇ Gly₁₈ Cys₁₉ Xaa₂₀ Xaa₂₁ (SEQ ID NO:93), or a pharmaceutically acceptable salt thereof; wherein
Xaa₁ is BE or is absent;
Xaa₂ is BK or is absent;
Xaa₃ is Asn or is absent;
Xaa₄ is Ser or is absent;
Xaa₅ is Ser, Asn, Ile, or is absent;
Xaa₆ is Tyr, Asp, 4-fluorophenylalanine ((4-F)Phe), or is absent;
Xaa₇ is Cys, cystathionine (Cth), allylglycine (Ag), Hag, or Asp;
Xaa₈ is Cys, cystathionine (Cth), penicillamine (Pen), or allylglycine (Ag);
Xaa₉ is Glu, Asp, Ser, Thr, or Gln;
Xaa₁₀ is Leu, cyclohexylalanine (Cha), Phe, or 4-fluorophenylalanine ((4-F)Phe);
Xaa₁₂ is Cys, allylglycine (Ag), Hag, or Val;
Xaa₁₄ is Pro, Val, sarcosine (Sar), Leu, or Hydroxyproline (OH-Pro);
Xaa₁₇ is Tyr, Thr, cyclohexylalanine (Cha), 4-fluorophenylalanine ((4-F)Phe), Phe, or Ala;
Xaa₂₀ is Tyr, Leu, 4-fluorophenylalanine ((4-F)Phe), cyclohexylalanine (Cha), D-Tyr, N-Methyl Tyr (Nme-Tyr) or is absent;
Xaa₂₁ is absent or Asn;
   wherein at least one Xaa is BE, BK, (4-F)Phe, Cth, Ag, Hag, Pen, Cha, Sar, or OH-Pro; and
   wherein Xaa₇ and Xaa₁₂ are both Ag and a dicarba bond is present between Ag₇ and Ag₁₂;
   Xaa₇ and Xaa₁₂ are both Cys and a disulfide bond is present between Cys₇ and Cys₁₂;
   Xaa₈ is Cys and a disulfide bond is present between Cys₈ and Cys₁₆;
   Xaa₇ is Cth and a bond is present between Cth₇ and Cys₁₂;
   Xaa₈ is Cth and a bond is present between Cth₈ and Cys₁₆;
   a disulfide bond is present between Cys₁₁ and Cys₁₉; or
   any combination thereof.

In some embodiments, the peptide is isolated. In other embodiments, the peptide is purified.

In some embodiments, a pharmaceutically acceptable salt of the peptide is provided. In some instances, the pharmaceutically acceptable salt is a chloride, acetate, phosphate or sulfate salt.

In some embodiments, the N-terminus of the peptides described herein is acetylated or capped. This modification may provide enhanced stability to the peptide. In other embodiments, the N-terminal peptide is a cysteine residue that has been modified into an imidazolidinone derivative.

The peptides disclosed herein may also be used for detection opportunities or colon cancer treatment. In the case of using the peptides in detection situations, a linker is conjugated to the N-terminus. The linker may then be conjugated to a dye, or may be conjugated to a dye prior to bonding with the peptide. One skilled in the art would recognize a peptide conjugated dye would be useful in detection of peptide binding interactions.

Another embodiment includes conjugating the peptides disclosed herein to a toxin by way of a linker. A toxin conjugated and coated with a peptide or pharmaceutically acceptable salt as described herein would be useful as a colon cancer treatment. Such a formulation would result in systemic circulation for a long-acting treatment. One skilled in the art would recognize the use of a long-acting peptide or pharmaceutically acceptable salt thereof as described herein in the treatment of colon cancer.

### Production of Peptides

In one embodiment, peptides or precursor peptides of the invention can be produced recombinantly in any known protein expression system, including, without limitation, bacteria (e.g., E. coli or Bacillus subtilis), insect cell systems (e.g., Drosophila Sf9 cell systems), yeast cell systems (e.g., S. cerevisiae, S. saccharomyces) or filamentous fungal expression systems, or animal cell expression systems (e.g., mammalian cell expression systems). If the peptide or variant peptide is to be produced recombinantly, e.g., E. coli, the nucleic acid molecule encoding the peptide may also encode a leader sequence that permits the secretion of the mature peptide from the cell. Thus, the sequence encoding the peptide can include the pre sequence and the pro sequence of, for example, a naturally-occurring bacterial heat-stable enterotoxin (ST) peptide. The secreted, mature peptide can be purified from the culture medium.

The sequence encoding a peptide described herein can be inserted into a vector capable of delivering and maintaining the nucleic acid molecule in a bacterial cell. The DNA molecule may be inserted into an autonomously replicating vector (suitable vectors include, for example, pGEM3Z and pcDNA3, and derivatives thereof). The vector nucleic acid may be a bacterial or bacteriophage DNA such as bacteriophage lambda or M13 and derivatives thereof. Construction of a vector containing a nucleic acid described herein can be followed by transformation of a host cell such as a bacterium. Suitable bacterial hosts include but are not limited to, E. coli, B. subtilis, Pseudomonas and Salmonella. The genetic construct also includes, in addition to the encoding nucleic acid molecule, elements that allow expression, such as a promoter and regulatory sequences. The expression vectors may contain transcriptional control sequences that control transcriptional initiation, such as promoter, enhancer, operator, and repressor sequences. A variety of transcriptional control sequences are well known to those in the art. The expression vector can also include a translation regulatory sequence (e.g., an untranslated 5′ sequence, an untranslated 3′ sequence, or an internal ribosome entry site). The vector can be capable of autonomous replication or it can integrate into host DNA to ensure stability during peptide production.

The protein coding sequence that includes a peptide described herein can also be fused to a nucleic acid encoding a peptide affinity tag, e.g., glutathione S-transferase (GST), maltose E binding protein, protein A, FLAG tag, hexa-histidine, myc tag or the influenza HA tag, in order to facilitate purification. The affinity tag or reporter fusion joins the reading frame of the peptide of interest to the reading frame of the gene encoding the affinity tag such that a translational fusion is generated. Expression of the fusion gene results in translation of a single peptide that includes both the peptide of interest and the affinity tag. In some instances where affinity tags are utilized, DNA sequence encoding a protease recognition site will be fused between the reading frames for the affinity tag and the peptide of interest.

Genetic constructs and methods suitable for production of immature and mature forms of the peptides and variants described herein in protein expression systems other than bacteria, and well known to those skilled in the art, can also be used to produce peptides in a biological system.

In some embodiments, peptides may be chemically produced. Peptides can be synthesized by a number of different methods including solution and solid phase synthesis using traditional BOC or FMOC protection. For example, the peptide can be synthesized on 2-Chlorotritylchloride or Wang resin using consecutive amino acid couplings. Various protecting groups can be used including, without limitation, the following protecting groups: Fluorenylmethyloxycarbonyl or tert-butyloxycarbonyl (alpha-amino groups, N-terminus); trityl or tert-butyl (thiol groups of Cys); tert-butyl (γ-carboxyl of glutamic acid and the hydroxyl group of threonine, if present); trityl (β-amid function of the asparagine side chain and the phenolic group of tyrosine, if present); trityl or tert-butyldimethylsilyl (hydroxygroup of serine, if present) and tert-Butyloxycarbonyl (N-terminus prior to subsequent side chain modifications). Coupling, deprotecting and cleavage can be effected through various methods. In some embodiments, coupling is effected with DIC and HOBt in the presence of a tertiary amine, and the peptide can be deprotected and cleaved from the solid support in using cocktail K (trifluoroacetic acid 81%, phenol 5%, thioanisole 5%, 1,2-ethanedithiol 2.5%, water 3%, dimethylsulphide 2%, ammonium iodide 1.5% w/w). The peptide can be isolated through various methods. In one embodiment, after removal of trifluoroacetic acid and other volatiles the peptide can be precipitated using an organic solvent. Disulfide bonds between Cys residues can be formed using various methods. In one embodiment, disulfide bonds between Cys residue can be formed using dimethyl sulfoxide (Tam et al. (1991) J. Am. Chem. Soc. 113:6657-62) or using an air oxidation strategy. The resulting peptide can be purified through various methods including, without limitation, by reverse-phase chromatography and lyophilized.

Peptides can be made, isolated or used either in form of the free base or as pharmaceutically acceptable salts thereof. Examples of salts include, without limitation, acetate, chloride, sulfate and phosphate salts of the peptide.

### Compositions of Peptides and GC-C Receptor Agonists

In another aspect, compositions are provided wherein the peptides, alone or in combination, can be combined with any pharmaceutically acceptable carrier or medium. In some embodiments, the peptide or pharmaceutically acceptable salt thereof can be formulated into a pharmaceutically acceptable composition. In other embodiments, the peptide or pharmaceutically acceptable salt thereof can be formulated into a non-pharmaceutically acceptable composition. The peptides can be combined with materials that do not produce an adverse, allergic or otherwise unwanted reaction when administered to a patient. The carriers or mediums used can include solvents, dispersants, coatings, absorption promoting agents, controlled release agents, and one or more inert excipients (which include starches, polyols, granulating agents, microcrystalline cellulose (e.g., celphere, Celphere Beads®), diluents, lubricants, binders, disintegrating agents, and the like), etc. If desired, tablet dosages of the disclosed compositions may be coated by standard aqueous or nonaqueous techniques.

Examples of excipients for use as the pharmaceutically acceptable carriers and the pharmaceutically acceptable inert carriers and the aforementioned additional ingredients include, but are not limited to binders, fillers, disintegrants, lubricants, anti-microbial agents, and coating agents.

As used herein, the term “binder” refers to any pharmaceutically acceptable binder that may be used in the practice of the invention. Examples of pharmaceutically acceptable binders include, without limitation, a starch (e.g., corn starch, potato starch and pre-gelatinized starch (e.g., STARCH 1500® and STARCH 1500 LM®, sold by Colorcon, Ltd.) and other starches), maltodextrin, gelatin, natural and synthetic gums such as acacia, powdered tragacanth, guar gum, cellulose and its derivatives (e.g., methylcellulose, hydroxyethyl cellulose, hydroxyethyl methylcellulose, hydroxypropyl cellulose and hydroxypropyl methylcellulose (hypromellose), ethyl cellulose, cellulose acetate, carboxymethyl cellulose calcium, sodium carboxymethyl cellulose, carboxymethylcellulose, powdered cellulose, microfine cellulose, microcrystalline cellulose (e.g. AVICEL™, such as, AVICEL-PH-101™, -103™ and -105™, sold by FMC Corporation, Marcus Hook, PA, USA)), polyvinyl alcohol, polyvinyl pyrrolidone (e.g., polyvinyl pyrrolidone K30), and mixtures thereof.

Examples of binders that may be particularly used in pharmaceutical compositions include polyvinyl alcohol, polyvinylpyrrolidone (povidone), a starch, maltodextrin or a cellulose ether (such as, for example, methylcellulose, ethylcellulose, carboxymethylcellulose, hydroxyethyl cellulose, hydroxyethyl methylcellulose, hydroxypropyl cellulose and hydroxypropyl methylcellulose).

As used herein, the term “filler” refers to any pharmaceutically acceptable filler that may be used in the practice of the invention. Examples of pharmaceutically acceptable fillers include, without limitation, talc, calcium carbonate (e.g., granules or powder), dibasic calcium phosphate, tribasic calcium phosphate, calcium sulfate (e.g., granules or powder), microcrystalline cellulose (e.g., Avicel PH101 or Celphere CP-305), microfine cellulose, powdered cellulose, dextrates, kaolin, mannitol, silicic acid, sorbitol, starch (e.g., Starch 1500), pre-gelatinized starch, lactose, glucose, fructose, galactose, trehalose, sucrose, maltose, isomalt, raffinose, maltitol, melezitose, stachyose, lactitol, palatinite, xylitol, myoinositol, and mixtures thereof.

Examples of pharmaceutically acceptable fillers that may be particularly used for coating the peptides include, without limitation, talc, microcrystalline cellulose (e.g., Avicel PH101 or Celphere CP-305), powdered cellulose, dextrates, kaolin, mannitol, silicic acid, sorbitol, starch, pre-gelatinized starch, lactose, glucose, fructose, galactose, trehalose, sucrose, maltose, isomalt, dibasic calcium phosphate, raffinose, maltitol, melezitose, stachyose, lactitol, palatinite, xylitol, mannitol, myoinositol, and mixtures thereof.

As used herein, the term “additives” refers to any pharmaceutically acceptable additive. Pharmaceutically acceptable additives include, without limitation, disintegrants, dispersing additives, lubricants, glidants, antioxidants, coating additives, diluents, surfactants, flavoring additives, humectants, absorption promoting additives, controlled release additives, anti-caking additives, anti-microbial agents (e.g., preservatives), colorants, desiccants, plasticizers and dyes. As used herein, an “excipient” is any pharmaceutically acceptable additive, filler, binder or agent.

Compositions of the present invention may also optionally include other therapeutic ingredients, anti-caking agents, preservatives, sweetening agents, colorants, flavors, desiccants, plasticizers, dyes, glidants, anti-adherents, anti-static agents, surfactants (wetting agents), anti-oxidants, film-coating agents, and the like. Any such optional ingredient must be compatible with the compound described herein to insure the stability of the formulation. The composition may contain other additives as needed, including for example lactose, glucose, fructose, galactose, trehalose, sucrose, maltose, raffinose, maltitol, melezitose, stachyose, lactitol, palatinite, starch, xylitol, mannitol, myoinositol, and the like, and hydrates thereof, and amino acids, for example alanine, glycine and betaine, and peptides and proteins, for example albumen.

The compositions can include, for example, various additional solvents, dispersants, coatings, absorption promoting additives, controlled release additives, and one or more inert additives (which include, for example, starches, polyols, granulating additives, microcrystalline cellulose, diluents, lubricants, binders, disintegrating additives, and the like), etc. If desired, tablet dosages of the disclosed compositions may be coated by standard aqueous or non-aqueous techniques. Compositions can also include, for example, anti-caking additives, preservatives, sweetening additives, colorants, flavors, desiccants, plasticizers, dyes, and the like.

Suitable disintegrants include, for example, agar-agar, calcium carbonate, microcrystalline cellulose, croscarmellose sodium, crospovidone, povidone, polacrilin potassium, sodium starch glycolate, potato or tapioca starch, other starches, pre-gelatinized starch, clays, other algins, other celluloses, gums, and mixtures thereof.

Suitable lubricants include, for example, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil and soybean oil), zinc stearate, ethyl oleate, ethyl laurate, agar, syloid silica gel (AEROSIL 200, W. R. Grace Co., Baltimore, MD USA), a coagulated aerosol of synthetic silica (Evonik Degussa Co., Plano, TX USA), a pyrogenic silicon dioxide (CAB-O-SIL, Cabot Co., Boston, MA USA), and mixtures thereof.

Suitable glidants include, for example, leucine, colloidal silicon dioxide, magnesium trisilicate, powdered cellulose, starch, talc, and tribasic calcium phosphate.

Suitable anti-caking additives include, for example, calcium silicate, magnesium silicate, silicon dioxide, colloidal silicon dioxide, talc, and mixtures thereof.

Suitable anti-microbial additives that may be used, e.g., as a preservative for the peptides compositions, include, for example, benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenoxyethanol, phenylmercuric acetate, phenylmercuric nitrate, potassium sorbate, propylparaben, sodium benzoate, sodium dehydroacetate, sodium propionate, sorbic acid, thimersol, thymo, and mixtures thereof.

Suitable antioxidants include, for example, BHA (butylated hydroxyanisole), BHT (butylated hydroxytoluene), vitamin E, propyl gallate, ascorbic acid and salts or esters thereof, tocopherol and esters thereof, alpha-lipoic acid and beta-carotene.

Suitable coating additives include, for example, sodium carboxymethyl cellulose, cellulose acetate phthalate, ethylcellulose, gelatin, pharmaceutical glaze, hydroxypropyl cellulose, hydroxypropyl methylcellulose, hydroxypropyl methyl cellulose phthalate, methylcellulose, polyethylene glycol, polyvinyl acetate phthalate, shellac, sucrose, titanium dioxide, carnauba wax, microcrystalline wax, and mixtures thereof. Suitable protective coatings include Aquacoat (e.g., Aquacoat Ethylcellulose Aquaeous Dispersion, 15% w/w, FMC Biopolymer, ECD-30), Eudragit (e.g., Eudragit E PO PE-EL, Roehm Pharma Polymers) and Opadry (e.g Opadry AMB dispersion, 20% w/w, Colorcon).

In certain embodiments, suitable additives for the peptides composition include one or more of sucrose, talc, magnesium stearate, crospovidone or BHA.

The compositions of the present invention can also include other excipients, agents, and categories thereof including but not limited to L-histidine, Pluronic®, Poloxamers (such as Lutrol® and Poloxamer 188), ascorbic acid, glutathione, permeability enhancers (e.g., lipids, sodium cholate, acylcarnitine, salicylates, mixed bile salts, fatty acid micelles, chelators, fatty acid, surfactants, medium chain glycerides), protease inhibitors (e.g., soybean trypsin inhibitor, organic acids), pH lowering agents and absorption enhancers effective to promote bioavailability (including but not limited to those described in U.S. Pat. Nos. 6,086,918 and 5,912,014), materials for chewable tablets (like dextrose, fructose, lactose monohydrate, lactose and aspartame, lactose and cellulose, maltodextrin, maltose, mannitol, microcrystalline cellulose and guar gum, sorbitol crystalline); parenterals (like mannitol and povidone); plasticizers (like dibutyl sebacate, plasticizers for coatings, polyvinylacetate phthalate); powder lubricants (like glyceryl behenate); soft gelatin capsules (like sorbitol special solution); spheres for coating (like sugar spheres); spheronization agents (like glyceryl behenate and microcrystalline cellulose); suspending/gelling agents (like carrageenan, gellan gum, mannitol, microcrystalline cellulose, povidone, sodium starch glycolate, xanthan gum); sweeteners (like aspartame, aspartame and lactose, dextrose, fructose, honey, maltodextrin, maltose, mannitol, molasses, sorbitol crystalline, sorbitol special solution, sucrose); wet granulation agents (like calcium carbonate, lactose anhydrous, lactose monohydrate, maltodextrin, mannitol, microcrystalline cellulose, povidone, starch), caramel, carboxymethylcellulose sodium, cherry cream flavor and cherry flavor, citric acid anhydrous, citric acid, confectioner's sugar, D&C Red No. 33, D&C Yellow #10 Aluminum Lake, disodium edetate, ethyl alcohol 15%, FD& C Yellow No. 6 aluminum lake, FD&C Blue #1 Aluminum Lake, FD&C Blue No. 1, FD&C blue no. 2 aluminum lake, FD&C Green No. 3, FD&C Red No. 40, FD&C Yellow No. 6 Aluminum Lake, FD&C Yellow No. 6, FD&C Yellow No. 10, glycerol palmitostearate, glyceryl monostearate, indigo carmine, lecithin, manitol, methyl and propyl parabens, mono ammonium glycyrrhizinate, natural and artificial orange flavor, pharmaceutical glaze, poloxamer 188, Polydextrose, polysorbate 20, polysorbate 80, polyvidone, pregelatinized corn starch, pregelatinized starch, red iron oxide, saccharin sodium, sodium carboxymethyl ether, sodium chloride, sodium citrate, sodium phosphate, strawberry flavor, synthetic black iron oxide, synthetic red iron oxide, titanium dioxide, and white wax.

In some embodiments, there is provided a pharmaceutical composition comprising a peptide described herein and one or more stabilizing agents selected from Mg²⁺, Ca²⁺, Zn²⁺, Mn²⁺, K⁺, Na⁺ or Al³⁺, a combination thereof, and/or a sterically hindered primary amine. In further embodiments, the agent is Mg²⁺, Ca²⁺ or Zn²⁺ or a combination thereof. In some embodiments, the cation is provided, without limitation, as magnesium acetate, magnesium chloride, magnesium phosphate, magnesium sulfate, calcium acetate, calcium chloride, calcium phosphate, calcium sulfate, zinc acetate, zinc chloride, zinc phosphate, zinc sulfate, manganese acetate, manganese chloride, manganese phosphate, manganese sulfate, potassium acetate, potassium chloride, potassium phosphate, potassium sulfate, sodium acetate, sodium chloride, sodium phosphate, sodium sulfate, aluminum acetate, aluminum chloride, aluminum phosphate or aluminum sulfate. In further embodiments, the cation is provided as magnesium chloride, calcium chloride, calcium phosphate, calcium sulfate, zinc acetate, manganese chloride, potassium chloride, sodium chloride or aluminum chloride. In other embodiments, the cation is provided as calcium chloride, magnesium chloride or zinc acetate.

In another embodiment, the stabilizing agent is a sterically hindered primary amine. In a further embodiment, the sterically hindered primary amine is an amino acid. In yet a further embodiment, the amino acid is a naturally-occurring amino acid. In a still further embodiment, the naturally-occurring amino acid is selected from the group consisting of: histidine, phenylalanine, alanine, glutamic acid, aspartic acid, glutamine, leucine, methionine, asparagine, tyrosine, threonine, isoleucine, tryptophan, glycine and valine; yet further, the naturally-occurring amino acid is leucine, isoleucine, alanine or methionine. In another embodiment, the sterically hindered primary amine is a non-naturally occurring amino acid (e.g., 1-aminocyclohexane carboxylic acid). In a further embodiment, the sterically hindered primary amine is cyclohexylamine, 2-methylbutylamine or a polymeric amine such as chitosan. In another embodiment, one or more sterically hindered primary amines may be used in a composition.

In some cases, the sterically hindered primary amine has the formula:

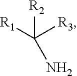

wherein R₁, R₂ and R₃ are independently selected from: H, C(O)OH, C₁-C₆ alkyl, C₁-C₆ alkylether, C₁-C₆ alkylthioether, C₁-C₆ alkyl carboxylic acid, C₁-C₆ alkyl carboxylamide and alkylaryl, wherein any group can be singly or multiply substituted with: halogen or amino, and provided that no more than two of R₁, R₂ and R₃ are H. In another embodiment, no more than one of R₁, R₂ and R₃ is H.

In other embodiments, there is provided a pharmaceutical composition comprising a pharmaceutically acceptable carrier, peptide, a cation selected from Mg²⁺, Ca²⁺, Zn²⁺, Mn²⁺, K⁺, Na⁺ or Al³⁺, or a mixture thereof, and a sterically hindered primary amine. In one embodiment, the cation is Mg²⁺, Ca²⁺ or Zn²⁺ or a mixture thereof. In a further embodiment, the pharmaceutical composition further comprises a pharmaceutically acceptable binder and/or a pharmaceutically acceptable glidant, lubricant or additive that acts as both a glidant and lubricant and/or an antioxidant. In some embodiments, the pharmaceutical composition is applied to a carrier. In some embodiments, the carrier is a filler.

In some cases the molar ratio of cation:sterically hindered primary amine: peptide in the aqueous solution applied to the carrier is 5-100:5-50:1. In some cases, the molar ratio of cation:sterically hindered primary amine may be equal to or greater than 2:1 (e.g., between 5:1 and 2:1). Thus, in some cases the molar ratio of cation:sterically hindered primary amine: peptide applied to the carrier is 100:50:1, 100:30:1, 80:40:1, 80:30:1, 80:20:1, 60:30:1, 60:20:1, 50:30:1, 50:20:1, 40:20:1, 20:20:1, 10:10:1, 10:5:1 or 5:10:1. When binder, e.g., methylcellulose, is present in the GC-C agonist peptide solution applied to the carrier it can be present at 0.5%-2.5% by weight (e.g., 0.7%-1.7% or 0.7%-1% or 1.5% or 0.7%).

It has been found that a cation selected from Mg²⁺, Ca²⁺, Zn²⁺, Mn²⁺, K⁺, Na⁺ and Al³⁺ is useful for suppressing the formation of an oxidation product of GC-C receptor agonist polypeptides during storage. It has also been found that a sterically hindered primary amine is useful for suppressing the formation of a formaldehyde imine adduct (“formaldehyde imine product”) of the GC-C receptor agonist polypeptides during storage. Thus, the GC-C receptor agonist polypeptide formulations comprising a cation selected from Mg²⁺, Ca²⁺, Zn²⁺, Mn²⁺, K⁺, Na⁺ or Al³⁺—for example, a divalent cation selected from Zn²⁺, Mg²⁺ and Ca²⁺—and/or a sterically hindered primary amine, such as an amino acid, have a sufficient shelf life (as measured by chromatographic purity and/or by a weight/weight assay) for manufacturing, storing and distributing the drug. Further, while the presence of a sterically hindered amine alone can increase the formation of a hydrolysis product of GC-C receptor agonist polypeptides during storage, the combination of a sterically hindered primary amine and a cation, e.g., but not limited to, the combination of leucine and Ca²⁺, suppresses the formation of the hydrolysis product of the GC-C receptor agonist polypeptide as well as the oxidation product of GC-C receptor agonist polypeptide during storage, leading to an even greater overall stability as determined by a weight/weight assay and/or by chromatographic purity.

In a further embodiment, the pharmaceutical composition further comprises a pharmaceutically acceptable binder or additive, and/or a pharmaceutically acceptable glidant, lubricant or additive that acts as both a glidant and lubricant and/or an antioxidant.

Suitable pharmaceutical compositions in accordance with the invention will generally include an amount of the active compound(s) with an acceptable pharmaceutical diluent or excipient, such as a sterile aqueous solution, to give a range of final concentrations, depending on the intended use. The techniques of preparation are generally well known in the art, as exemplified by Remington's Pharmaceutical Sciences (18th Edition, Mack Publishing Company, 1995).

For treatment of gastrointestinal disorders, the peptides described herein are administered orally or rectally, e.g., as a tablet, capsule, sachet containing a predetermined amount of the active ingredient pellet, gel, paste, syrup, bolus, electuary, slurry, powder, lyophilized powder, granules, as a solution or a suspension in an aqueous liquid or a non-aqueous liquid; as an oil-in-water liquid emulsion or a water-in-oil liquid emulsion, via a liposomal formulation (see, e.g., EP 736299) or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein. The peptides can be co-administered with other agents used to treat gastrointestinal disorders including but not limited to the agents described herein.

In another aspect, suitable pharmaceutical compositions may comprise one or more other therapeutic agents. Such therapeutic agents include, without limitation, analgesic agents; anti-secretory agents, including proton pump inhibitors, acid pump antagonists, H2 receptor antagonists; PDE5 inhibitors; ODC inhibitors; GABA-B antagonists; bile acid sequestrants; prokinetic and promotility agents; antidepressants; antibiotics; antiemetics; opioids; and mucosal-protecting agents.

### Methods of Treatment

In various embodiments, the peptides or the pharmaceutically acceptable salts thereof may be useful in methods for colon cleansing treatments or the treatment of gastrointestinal disorders. In some embodiments, the peptides or pharmaceutically acceptable salts thereof may be useful in a method for cleansing the colon prior to a colonoscopy or surgical procedure. In further embodiments, the peptides may be used to prepare subjects for colonoscopy treatment. In some embodiments, the peptides or pharmaceutically acceptable salts may be used to prepare subjects for surgery, such as bowel surgery. In other embodiments, the peptides may be used to treat gastrointestinal disorders, visceral disorders, colon cancer, Hereditary Nonpolyposis Colorectal Cancer (HNPCC), i.e. Lynch syndrome, gastroparesis (GP), polyps, pain, general abdominal pain, post-operative ileus, opioid-induced constipation, functional dyspepsia, diverticular disease including including but not limited to SUDD (symptomatic uncomplicated diverticular disease) and SCAD (segmental colitis associated with diverticulosis), diverticulosis, diarrhea-predominant irritable bowel syndrome, pain associated with irritable bowel syndrom (IBS), ulcerative colitis, ulcerative proctitis, Crohn's Disease, inflammatory bowel disease (IBD), chronic or acute radiation protopathy, rectal pain, chronic proctalgia, proctalgia fugax, anal pain, chronic anal fissure, post-operative anal pain, overactive bladder syndrome, stress incontinence, interstitial cystitis, bladder pain syndrome, colorectal cancer, pain associated with cancer, general pelvic pain, endometriosis, orchialgia, chronic prostatitis, prostatodynia, vulvodynia, urethral syndrome, penile pain, perianal pain and other gastrointestinal and visceral disorders. In some embodiments of the invention, a method of treatment is provided for gastrointestinal disorders. In one embodiment, the gastrointestinal disorder is colon cancer or polyps. In another embodiment, the gastrointestinal disorder is Hereditary Nonpolyposis Colorectal Cancer (HNPCC), i.e. Lynch syndrome. In another embodiment, the gastrointestinal disorder is gastrointestinal pain. In a further embodiment, the gastrointestinal disorder is visceral or abdominal pain or pain associated with cancer. In another embodiment, the gastrointestinal disorder is rectal cancer. In another embodiment, the gastrointestinal disorder is functional dyspepsia.

In one embodiment, a method is provided for cleansing the colon of a subject in preparation for a colonoscopy procedure comprising administering to the subject an effective dose of a colon cleansing composition, wherein the colon cleansing composition comprises a pharmaceutically acceptable excipient, diluent or carrier, and a peptide or a pharmaceutically acceptable salt thereof, wherein the peptide or a pharmaceutically acceptable salt thereof comprises the amino acid sequence:
Xaa₁ Xaa₂ Xaa₃ Xaa₄ Xaa₅ Xaa₆ Xaa₇ Xaa₈ Xaa₉ Xaa₁₀ Xaa₁₁ Xaa₁₂ Xaa₁₃ Xaa₁₄ Ala₁₅ Xaa₁₆ Xaa₁₇ Gly₁₈ Xaa₁₉ Xaa₂₀ Xaa₂₁ (SEQ ID NO: 1), or a pharmaceutically acceptable salt thereof; wherein
Xaa₁ is BE or is absent;
Xaa₂ is BK, Asn or is absent;
Xaa₃ is Asn, Ser or is absent;
Xaa₄ is Ser or is absent;
Xaa₅ is Ser, Asn, Ile, BE or is absent;
Xaa₆ is Tyr, Asp, 4-fluorophenylalanine ((4-F)Phe), BK or is absent;
Xaa₇ is Cys, cystathionine (Cth), allylglycine (Ag), Hag, or Asp;
Xaa₈ is Cys, cystathionine (Cth), penicillamine (Pen), or allylglycine (Ag);
Xaa₉ is Glu, Asp, Ser, Thr, or Gln;
Xaa₁₀ is Leu, cyclohexylalanine (Cha), Phe, or 4-fluorophenylalanine ((4-F)Phe);
Xaa₁₁ is Cys, Ag, or penicillamine (Pen);
Xaa₁₂ is Cys, allylglycine (Ag), Hag, Cth, Dpr, or Val;
Xaa₁₃ is Asn or Leu;
Xaa₁₄ is Pro, Val, sarcosine (Sar), Leu, or Hydroxyproline (OH-Pro);
Xaa₁₆ is Cys, Ag, Pen or Cth;
Xaa₁₇ is Tyr, Thr, cyclohexylalanine (Cha), 4-fluorophenylalanine ((4-F)Phe), Phe, Ser, or Ala;
Xaa₁₉ is Cys, Ag or Pen;
Xaa₂₀ is Tyr, Leu, 4-fluorophenylalanine ((4-F)Phe), cyclohexylalanine (Cha), D-Tyr, N-Methyl Tyr (Nme-Tyr) or is absent;
Xaa₂₁ is absent or Asn;
   wherein at least one Xaa is BE, BK, (4-F)Phe, Cth, Ag, Hag, Pen, Cha, Sar, Dpr, or OH-Pro and wherein the peptide contains a covalent bond between Xaa₇ and Xaa₁₂, Xaa₈ and Xaa₁₆, and Xaa₁₁ and Xaa₁₉.

In another embodiment, a method is provided for cleansing the colon of a subject in preparation for a colonoscopy procedure comprising administering to the subject an effective dose of a colon cleansing composition, wherein the colon cleansing composition comprises a pharmaceutically acceptable excipient, diluent or carrier, and a peptide or a pharmaceutically acceptable salt thereof, wherein the peptide or a pharmaceutically acceptable salt thereof comprises the amino acid sequence:

(SEQ ID NO: 2)
H-Ag Cys Glu Leu Cys Ag Asn Pro Ala Cys Thr Gly Cys Tyr-NH₂;
(SEQ ID NO: 3)
C12-BE BK Asn Ser Ser Tyr Cys Cys Glu Leu Cys Cys Asn Pro Ala Cys
Thr Gly Cys Tyr-NH₂;
(SEQ ID NO: 4)
C16-BE BK Asn Ser Ser Tyr Cys Cys Glu Leu Cys Cys Asn Pro Ala Cys
Thr Gly Cys Tyr-NH₂;
(SEQ ID NO: 5)
C12-BE BK Asn Ser Ser Tyr Cys Cys Glu Leu Cys Cys Asn Pro Ala Cys
Thr Gly Cys Tyr-COOH;
(SEQ ID NO: 6)
C14-BE BK Asn Ser Ser Tyr Cys Cys Glu Leu Cys Cys Asn Pro Ala Cys
Thr Gly Cys Tyr-COOH;
(SEQ ID NO: 7)
C16-BE BK Asn Ser Ser Tyr Cys Cys Glu Leu Cys Cys Asn Pro Ala Cys
Thr Gly Cys Tyr-COOH;
(SEQ ID NO: 8)
H-Cys Ag Glu Leu Cys Cys Asn Pro Ala Ag Thr Gly Cys Tyr-NH₂;
(SEQ ID NO: 9)
H-Asn Asp Asp Ag Glu Leu Cys Val Asn Val Ala Ag Thr Gly Cys Leu-
NH₂;
(SEQ ID NO: 10)
H-Asn Asp Asp Cys Glu Leu Ag Val Asn Val Ala Cys Thr Gly Ag Leu-
NH₂;
(SEQ ID NO: 11)
C18-BE BK Asn Ser Ser Tyr Cys Cys Glu Leu Cys Cys Asn Pro Ala Cys
Thr Gly Cys Tyr-COOH;
(SEQ ID NO: 12)
H-Ag Cys Glu Leu Cys Ag Asn Pro Ala Cys Thr Gly Cys-NH₂;
(SEQ ID NO: 13)
H-Ag Cys Glu Leu Cys Ag Asn Pro Ala Cys Ala Gly Cys-NH₂;
(SEQ ID NO: 14)
H-Ag Cys Glu Phe Cys Ag Asn Pro Ala Cys Thr Gly Cys Tyr-NH₂;
(SEQ ID NO: 15)
H-Ag Cys Glu (4-F)Phe Cys Ag Asn Pro Ala Cys Thr Gly Cys-NH₂;
(SEQ ID NO: 16)
H-Ag Cys Glu (4-F)Phe Cys Ag Asn Pro Ala Cys Thr Gly Cys Tyr-NH₂;
(SEQ ID NO: 17)
H-Ag Cys Glu (4-F)Phe Cys Ag Asn Pro Ala Cys Thr Gly Cys (4-F)Phe-
NH₂;
(SEQ ID NO: 18)
H-Cys Cys Glu Leu Ag Cys Asn Pro Ala Cys Thr Gly Ag Tyr-NH₂;
(SEQ ID NO: 19)
H-Ag Cys Glu Phe Cys Ag Asn Pro Ala Cys Thr Gly Cys-NH₂;
(SEQ ID NO: 20)
H-Ag Cys Glu Phe Cys Ag Asn Pro Ala Cys Thr Gly Cys (4-F)Phe-NH₂;
(SEQ ID NO: 21)
H-Cys Cys Glu (4-F)Phe Cys Cys Asn Pro Ala Cys Thr Gly Cys Tyr-NH₂;
(SEQ ID NO: 22)
H-Cys Cys Glu (4-F)Phe Cys Cys Asn Pro Ala Cys Thr Gly Cys (4-F)Phe-
NH₂;
(SEQ ID NO: 23)
H-Ag Cys Glu Leu Cys Ag Asn Pro Ala Cys Thr Gly Cys (4-F)Phe-NH₂;
(SEQ ID NO: 24)
H-(4-F)Phe Ag Cys Glu Leu Cys Ag Asn Pro Ala Cys Thr Gly Cys (4-
F)Phe-NH₂;
(SEQ ID NO: 25)
H-Ag Cys Glu Leu Cys Ag Asn Pro Ala Cys Ala Gly Cys Tyr-NH₂;
(SEQ ID NO: 26)
H-Ag Cys Glu Leu Cys Ag Asn Pro Ala Cys Tyr Gly Cys Tyr-NH₂;
(SEQ ID NO: 27)
H-Ag Cys Glu Leu Cys Ag Asn Pro Ala Cys Phe Gly Cys Tyr-NH₂;
(SEQ ID NO: 28)
H-Ag Cys Glu Leu Cys Ag Asn Pro Ala Cys (4-F)Phe Gly Cys Tyr-NH₂;
(SEQ ID NO: 29)
H-Ag Cys Glu Leu Cys Ag Asn Pro Ala Cys Thr Gly Cys Leu-NH₂;
(SEQ ID NO: 30)
H-Ag Cys Glu Leu Cys Ag Asn Pro Ala Cys Thr Gly Cys Cha-NH₂;
(SEQ ID NO: 31)
H-Ag Cys Glu Cha Cys Ag Asn Pro Ala Cys Thr Gly Cys Tyr-NH₂;
(SEQ ID NO: 32)
H-Ag Cys Glu Leu Cys Ag Asn Pro Ala Cys Thr Gly Cys D-Tyr-NH₂;
(SEQ ID NO: 33)
H-Ag Pen Glu Leu Cys Ag Asn Pro Ala Pen Thr Gly Cys Tyr-NH₂;
(SEQ ID NO: 34)
H-Ag Cys Glu Leu Cys Ag Asn Pro Ala Cys Thr Gly Cys Leu Asn-NH₂;
(SEQ ID NO: 35)
H-Ag Cys Glu Leu Cys Ag Asn Pro Ala Cys Thr Gly Cys Tyr Asn-NH₂;
(SEQ ID NO: 36)
H-Asp Ag Cys Glu Leu Cys Ag Asn Pro Ala Cys Thr Gly Cys Tyr-NH₂;
(SEQ ID NO: 37)
H-Ile Asp Ag Cys Glu Leu Cys Ag Asn Pro Ala Cys Thr Gly Cys Tyr-
NH₂;
(SEQ ID NO: 38)
H-Ag Cys Glu Leu Cys Ag Asn OH-Pro Ala Cys Thr Gly Cys Tyr-NH₂;
(SEQ ID NO: 39)
H-Ag Cys Glu Leu Cys Ag Asn OH-Pro Ala Cys Thr Gly Cys-NH₂;
(SEQ ID NO: 40)
H-Ag Cys Glu Leu Pen Ag Asn Pro Ala Cys Thr Gly Pen Tyr-NH₂;
(SEQ ID NO: 41)
H-Ag Cys Glu Leu Pen Ag Asn Pro Ala Cys Thr Gly Pen Tyr-NH₂;
(SEQ ID NO: 42)
H-Ag Cys Glu Leu Cys Ag Asn Pro Ala Cys Tyr Gly Cys Nme-Tyr-NH₂;
(SEQ ID NO: 43)
H-Ag Cys Glu Leu Cys Ag Asn Pro Ala Cys Tyr Gly Cys-COOH;
(SEQ ID NO: 44)
H-Ag Cys Glu Leu Cys Ag Asn Pro Ala Cys Cha Gly Cys Tyr-NH₂;
(SEQ ID NO: 45)
H-Ag Cys Glu Leu Cys Ag Asn Leu Ala Cys Thr Gly Cys Tyr-NH₂;
(SEQ ID NO: 46)
H-Ag Cys Glu Leu Cys Ag Asn Sar Ala Cys Thr Gly Cys Tyr-NH₂;
(SEQ ID NO: 47)
H-Ag Cys Glu Leu Cys Ag Asn Val Ala Cys Thr Gly Cys Tyr-NH₂;
(SEQ ID NO: 48)
H-Ag Cys Glu Leu Cys Ag Asn Pro Ala Cys Tyr Gly Cys Nme-Tyr-NH₂;
(SEQ ID NO: 49)
H-Ag Cys Glu Leu Cys Ag Leu Pro Ala Cys Tyr Gly Cys-COOH;
(SEQ ID NO: 50)
H-Ag Cys Asp Leu Cys Ag Asn Pro Ala Cys Tyr Gly Cys-COOH;
(SEQ ID NO: 51)
H-Ag Cys Ser Leu Cys Ag Asn Pro Ala Cys Tyr Gly Cys-COOH;
(SEQ ID NO: 52)
H-Ag Cys Thr Leu Cys Ag Asn Pro Ala Cys Tyr Gly Cys-COOH;
(SEQ ID NO: 53)
H-Hag Cys Glu Leu Cys Hag Asn Pro Ala Cys Thr Gly Cys Tyr-NH₂;
(SEQ ID NO: 54)
H-Ag Cys Gln Leu Cys Ag Asn Pro Ala Cys Tyr Gly Cys-COOH;
(SEQ ID NO: 55)
H-Ag Cys Glu Leu Cys Ag Leu Pro Ala Cys Thr Gly Cys Tyr-NH₂;
(SEQ ID NO: 56)
Pent-Cys Glu Leu Cys Ag Asn Pro Ala Cys Tyr Gly Cys Tyr-NH₂;
(SEQ ID NO: 57)
H-Cys Cys Glu Leu Cys Ag Asn Pro Ala Cys Tyr Gly Ag-COOH;
(SEQ ID NO: 58)
Pent-Cys Glu Leu Cys Ag Asn Pro Ala Cys Tyr Gly Cys-COOH;
(SEQ ID NO: 59)
H-Hag Cys Glu Leu Cys Hag Asn Pro Ala Cys Tyr Gly Cys-COOH;
(SEQ ID NO: 60)
H-Ag Cys Glu Leu Cys Ag Asn Val Ala Cys Tyr Gly Cys Tyr-NH₂;
(SEQ ID NO: 61)
H-Ag Cys Glu Leu Cys Ag Asn Val Ala Cys Tyr Gly Cys-COOH;
(SEQ ID NO: 62)
H-Cys Cth Glu Leu Cys Cys Asn Pro Ala Cys Tyr Gly Cys Tyr-NH₂;
(SEQ ID NO: 63)
H-Cth Cys Glu Leu Cys Cys Asn Pro Ala Cys Tyr Gly Cys Tyr-NH₂;
(SEQ ID NO: 64)
H-Cth Cys Glu Leu Cys Cys Asn Pro Ala Cys Tyr Gly Cys-COOH;
(SEQ ID NO: 65)
H-Cys Cth Glu Leu Cys Cys Asn Val Ala Cys Tyr Gly Cys Tyr-NH₂;
(SEQ ID NO: 66)
H-Cys Cth Glu Leu Cys Cys Asn Val Ala Cys Thr Gly Cys Tyr-NH₂;
(SEQ ID NO: 67)
Ac-Cys Cth Glu Leu Cys Cys Asn Val Ala Cys Thr Gly Cys Tyr-NH₂;
(SEQ ID NO: 68)
Ac-Cys Cth Glu Leu Cys Cys Asn Pro Ala Cys Tyr Gly Cys Tyr-NH₂;
(SEQ ID NO: 69)
Ac-Cys Cth Glu Leu Cys Cys Asn Val Ala Cys Tyr Gly Cys-COOH;
(SEQ ID NO: 70)
H-Cys Cth Glu Leu Cys Cys Asn Val Ala Cys Tyr Gly Cys-COOH;
(SEQ ID NO: 71)
Ac-Cys Cth Glu Leu Cys Cys Asn Pro Ala Cys Tyr Gly Cys-COOH;
(SEQ ID NO: 72)
H-Cys Cys Glu Leu Cys Asn Val Ala Cth Tyr Gly Cys-COOH;
(SEQ ID NO: 73)
H-Cys Cys Glu Leu Cys Cys Asn Val Ala Cth Tyr Gly Cys Tyr-NH₂;
(SEQ ID NO: 74)
H-Cys Cys Glu Leu Cys Cys Asn Val Ala Cth Thr Gly Cys Tyr-NH₂;
(SEQ ID NO: 75)
H-Cys Cys Glu Leu Cys Cth Asn Val Ala Cys Tyr Gly Cys-COOH;
(SEQ ID NO: 76)
H-Cys Cys Glu Leu Cys Cth Asn Val Ala Cys Tyr Gly Cys Tyr-NH₂;
(SEQ ID NO: 77)
H-Cys Cys Glu Leu Cys Cth Asn Val Ala Cys Thr Gly Cys Tyr-NH₂;
(SEQ ID NO: 78)
4-Mepip-Cys Cth Glu Leu Cys Cys Asn Val Ala Cys Tyr Gly Cys-COOH;
(SEQ ID NO: 79)
H-Cys Cth Glu Leu Cys Cys Asn Val Ala Cys Thr Gly Cys-COOH;
(SEQ ID NO: 80)
H-Cys Cth Glu Leu Cys Cys Asn Val Ala Cys Thr Gly Cys-NH₂;
(SEQ ID NO: 81)
H-Cys Cth Glu Leu Cys Cys Asn Val Ala Cys Phe Gly Cys-COOH;
(SEQ ID NO: 82)
H-Ag Ag Glu Leu Ag Ag Asn Pro Ala Ag Thr Gly Ag Tyr-COOH;
(SEQ ID NO: 83)
H-Cys Cth Glu Leu Cys Cys Asn Ala Ala Cys Thr Gly Cys Tyr-NH₂;
(SEQ ID NO: 84)
H-Asp Cys Glu Leu Cys Dpr Asn Val Ala Cys Thr Gly Cys Tyr-NH₂;
(SEQ ID NO: 85)
H-Cys Cth Glu Leu Cys Cys Asn Val Ala Cys Ser Gly Cys Tyr-NH₂;
(SEQ ID NO: 86)
H-Cth Glu Leu Cys Ag Asn Val Ala Cys Thr Gly Cys Tyr-NH₂;
(SEQ ID NO: 87)
H-Ag Cys Glu Leu Cys Ag Asn Val Ala Cys Thr Gly Cys Tyr-NH₂;
(SEQ ID NO: 88)
H-Ag Cys Glu Leu Cys Ag Asn Pro Ala Cys Thr Gly Cys Tyr-NH₂;
(SEQ ID NO: 89)
H-Cth Cys Glu Leu Cys Cys Asn Val Ala Cys Thr Gly Cys-COOH;
(SEQ ID NO: 90)
H-Cth Cys Glu Leu Cys Cys Asn Val Ala Cys Thr Gly Cys Tyr-NH₂;
(SEQ ID NO: 91)
H-Cth Cys Glu Leu Cys Cys Asn Val Ala Cys Tyr Gly Cys Tyr-NH₂;
or
(SEQ ID NO: 92)
H-Cys Cys Glu Leu Cys Cys Asn Val Ala Cth Tyr Gly Cys-COOH.

In one embodiment, the peptides and compositions described herein provide a treatment for preparing a patient prior to a colonoscopy.

In some embodiments, the peptides and pharmaceutically acceptable salts described herein may be used as a method for colon cleansing in preparation for a colonoscopy procedure. In some embodiments, the method for colon cleansing comprises administering an effective first dose, such as between 5 μg and 100 mg of the peptide or pharmaceutically acceptable salt. On the following morning, an effective second dose is administered, such as between 5 μg and 100 mg of the peptide or pharmaceutically acceptable salt to substantially cleanse the colon. In other embodiments, the peptide or pharmaceutically acceptable salt thereof is administered in a single dose of between 5 μg and 200 mg.

The peptides and pharmaceutically acceptable salts described herein can be used alone or in combination therapy for the treatment, prevention or reduction of visceral pain associated with a gastrointestinal disorder, cancer, general pelvic pain, bladder pain, overactive bladder, endometriosis, orchialgia, chronic prostatitis, prostatodynia, vulvodynia, urethral syndrome, penile pain, and perianal pain or pain associated with another disorder as described herein.

The peptides and pharmaceutically acceptable salts described herein can be administered in combination with other agents. For example, the peptides can be administered with an analgesic peptide, soluble guanylate cyclase (sGC) stimulator or compound. The analgesic peptide or compound can be covalently attached to a peptide described herein or it can be a separate agent that is administered together with or sequentially with a peptide described herein in a combination therapy. The peptides and pharmaceutically acceptable salts described herein may also be administered in combination with other agents used to treat GI disorders including antidepressants, promotility or prokinetic agents, antiemetics, antibiotics, proton pump inhibitors, acid blockers (e.g., histamine H2 receptor antagonists), acid pump antagonists, PDE5 inhibitors, ODC inhibitors, GABA-B agonists, bile acid sequestrants, COX-2 inhibitors, NSAIDS, corticosteroids, opioids, beta-3 adrenergic receptor agonists, anti-cholinergic agents including but not limited to muscarinic receptor antagonists, tricyclic antidepressants and mucosal protecting agents.

In other embodiments, the therapeutic combinations of the peptides include Celecoxib, other nonsteroidal anti-inflammatory drugs (NSAIDS), including Sulidac and isomers, and phosphodiesterase (PDE) inhibitors and omithine decarboxylase (ODC) inhibitors (e.g. d,l-α-difluoromethylornithine DFMO) for colon polyps-sporadic and Lynch syndrome; Mesalamine or 5-aminosalicylic acid (5-asa), or steroids, Budesonide for inflammatory bowel disease (e.g. Crohns disease and ulcerative colitis); opioids, tramadol, and tramadol isomers and analogs for chronic pain, including cancer pain; or Eluxalodine.

In some embodiments, useful analgesic agents that may be used with the peptides described herein include Ca channel blockers (e.g., ziconotide), 5HT receptor antagonists (e.g., 5HT3, 5HT4 and 5HT1 receptor antagonists), 5HT4 agonists (e.g., tegaserod (Zelnorm®), mosapride, metoclopramide, zacopride, cisapride, renzapride, benzimidazolone derivatives such as BIMU 1 and BIMU 8, and lirexapride), 5HT1 agonists (e.g., sumatriptan and buspirone), opioid receptor agonists (e.g., loperamide, fedotozine, enkephalin pentapeptide, morphine, diphenyloxylate, frakefamide, trimebutine and fentanyl), CCK receptor agonists (e.g., loxiglumide and dexloxiglumide), NK1 receptor antagonists (e.g., aprepitant, vofopitant, ezlopitant, R-673 (Hoffmann-La Roche Ltd), SR-48968 and SR-14033, (Sanofi Synthelabo), CP-122,721 (Pfizer, Inc.), GW679769 (Glaxo Smith Kline) and TAK-637 (Takeda/Abbot)), NK2 receptor antagonists (e.g., nepadutant, saredutant, GW597599 (Glaxo Smith Kline), SR-144190 (Sanofi-Synthelabo) and UK-290795 (Pfizer Inc)), NK3 receptor antagonists (e.g., osanetant (SR-142801; Sanofi-Synthelabo), SR-241586 and talnetant), norepinephrine-serotonin reuptake inhibitors (NSRI) (e.g., milnacipran), vanilloid and cannabanoid receptor agonists, sialorphin and sialorphin-related peptides. Analgesic agents in the various classes are described in the literature.

In some embodiments, one or more other therapeutic agents may be used in combination with the peptides described herein. Such agents include antidepressants, promotility or prokinetic agents, antiemetics, antibiotics, proton pump inhibitors, acid blockers (e.g., histamine H2 receptor antagonists), acid pump antagonists, PDE5 inhibitors, ODC inhibitors, GABA-B agonists, bile acid sequestrants, opioids and mucosal protecting agents.

Examples of antidepressants include, without limitation, tricyclic antidepressants such as amitriptyline (Elavil®), desipramine (Norpramin®), imipramine (Tofranil®), amoxapine (Asendin®), nortriptyline; the selective serotonin reuptake inhibitors (SSRI's) such as paroxetine (Paxil®), fluoxetine (Prozac®), sertraline (Zoloft®), and citralopram (Celexa®); and others such as doxepin (Sinequan®) and trazodone (Desyrel®).

Examples of promotility and prokinetic agents include, without limitation, itopride, octreotide, bethanechol, metoclopramide (Reglan®), domperidone (Motilium®), erythromycin (and derivatives thereof) and cisapride (Propulsid®). An example of antiemetics includes, without limitation, prochlorperazine.

Examples of antibiotics that may be used include those that may be used to treat Heliobacter pylori infections, such as amoxicillin, tetracycline, metronidazole, or clarithromycin. Other antibiotics such as erythromycin and derivatives thereof may also be used in combination with the peptides described herein.

Examples of proton pump inhibitors include, without limitation, omeprazole (Prilosec®), esomeprazole (Nexium®), lansoprazole (Prevacid®), pantoprazole (Protonix®) and rabeprazole (Aciphex®). Examples of H2 receptor blockers include, without limitation, including cimetidine, ranitidine, famotidine and nizatidine. Examples of acid pump antagonists include, without limitation, revaprazan, CS-526 (J. Pharmacol. Exp. Ther. (2007) 323:308-317), PF-03716556 (J. Pharmacol. Exp. Ther. (2009) 328(2):671-9), and YH1885 (Drug Metab. Dispos. (2001) 29(1):54-9).

Examples of PDE5 inhibitors include, without limitation, avanafil, lodenafil, mirodenafil, sildenafil citrate, tadalafil, vardenafil and udenafil. GABA-B agonists include, without limitation, baclofen and XP19986 (CAS Registry No. 847353-30-4). Examples of bile acid sequestrants include, without limitation, GT102-279, cholestyramine, colesevelam, colesevelam hydrochloride, ursodeoxycholic acid, colestipol, colestilan, sevelamer, polydiallylamine cross-linked with epichlorohydrin, dialkylaminoalkyl derivatives of a cross-linked dextran, and N-(cycloalkyl)alkylamines. Examples of mucosal protecting agents include, without limitation, sucralfate (Carafate), teprenone, polaprezinc, cetraxate and bismuth subsalicyclate.

Combination therapy can be achieved by administering two or more active agents, e.g., a peptide or pharmaceutically acceptable salt described herein and another therapeutic peptide or compound, each of which is formulated and administered separately, or by administering two or more active agents in a single formulation. Other combinations are also encompassed by combination therapy. For example, two active agents can be formulated together and administered in conjunction with a separate formulation containing a third active agent. While the two or more active agents in the combination therapy can be administered simultaneously, they need not be. For example, administration of a first active agent (or combination of active agents) can precede administration of a second agent (or combination of agents) by minutes, hours, days, or weeks. Thus, the two or more active agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more active agents used in a combination therapy be present in within the patient's body at the same time, this need not be so.

### Dosage

The dose range for adult humans may be generally from 5 μg to 100 mg/day orally or rectally for the peptides and pharmaceutically acceptable salts described herein. Tablets, capsules, or other forms of presentation provided in discrete units may conveniently contain an amount of compound described herein which is effective at such dosage or as a multiple of the same, for instance, units containing 25 μg to 2 mg or around 100 μg to 1 mg. The precise amount of compound prescribed to a patient will be the responsibility of the attendant physician. However, the dose employed will depend on a number of factors, including the age and sex of the patient, the precise disorder being treated, and its severity.

In various embodiments, the dosage unit is administered with food at anytime of the day, without food at anytime of the day, with food after an overnight fast (e.g. with breakfast), at bedtime after a low fat snack. In one particular embodiment, the dosage unit is administered prior to or subsequent to food consumption (e.g., a meal). In a further embodiment, the dosage unit is administered approximately 15 minutes to 1 hour prior to food consumption. In various embodiments, the dosage unit is administered once a day, twice a day, three times a day, four times a day, five times a day or six times a day. In certain embodiments the dosage unit and daily dose are equivalent.

In some embodiments, a composition containing the peptides described herein is provided in a split dose. The split dose is administered the night before and the day of the colonoscopy. In other embodiments, the dosage is provided in a single dose the night or day before or on the day of the colonoscopy.

In combination therapy embodiments of the present invention, the precise amount of each of the two or more active ingredients in a dosage unit will depend on the desired dosage of each component. Thus, it can be useful to create a dosage unit that will, when administered according to a particular dosage schedule (e.g., a dosage schedule specifying a certain number of units and a particular timing for administration), deliver the same dosage of each component as would be administered if the patient was being treated with only a single component. In other circumstances, it might be desirable to create a dosage unit that will deliver a dosage of one or more components that is less than that which would be administered if the patient was being treated only with a single component. Finally, it might be desirable to create a dosage unit that will deliver a dosage of one or more components that is greater than that which would be administered if the patient was being treated only with a single component.

The pharmaceutical composition can include additional ingredients including but not limited to the active ingredients and excipients described herein. In certain embodiments, one or more therapeutic agents of the dosage unit may exist in an extended or control release formulation and additional therapeutic agents may not exist in extended release formulation. For example, a peptide or agonist described herein may exist in a controlled release formulation or extended release formulation in the same dosage unit with another agent that may or may not be in either a controlled release or extended release formulation. Thus, in certain embodiments, it may be desirable to provide for the immediate release of one or more of the agents described herein, and the controlled release of one or more other agents.

The present invention has been described with reference to certain exemplary embodiments thereof. However, it will be readily apparent to those skilled in the art that it is possible to embody the invention in specific forms other than those of the exemplary embodiments described above. This may be done without departing from the spirit of the invention. The exemplary embodiments are merely illustrative and should not be considered restrictive in any way. The scope of the invention is defined by the appended claims and their equivalents, rather than by the preceding description.

### EXAMPLES

### Example 1: cGMP Accumulation in T84 Cells for Analysis of GC-C Activity

For the cGMP assay, 2.0×10⁵ cells/mL of T84 cells were grown overnight in 96 well tissue culture plates. On the next day, the T84 cells were washed twice with 200 μL of DMEM+20 mM MES (pH 5) or DMEM+50 mM sodium bicarbonate (pH8) pr DMEM with no additive for pH 7. These buffers do not contain serum. After the second wash, the cells were incubated with 180 μL of 1 mM isobutylmethylxanthine (IBMX) in either the pH 5, 7 or 8 buffers for 10 minutes at 37° C. to inhibit any phosphodiesterase activity. The peptides were then diluted in either pH 5, 7 or 8 buffer to a 10× concentration. The peptide solution of 20 μL was diluted to a final volume of 200 μL with the T84 cells, bringing each peptide concentration to 1×. An eleven point curve analysis was conducted for each peptide, with final peptide concentrations tested in each assay, in nM: 10000, 3000, 1000, 300, 100, 30, 10, 3, 1, 0.3, 0.1.

There was no peptide control used to determine endogenous levels of cGMP. Peptides were incubated for 30 minutes at 37° C. After 30 minutes, the supernatants were removed and the cells were lysed with 200 μL of 0.1 M HCl. The cells were lysed for 30 minutes on ice. After 30 minutes, lysates were pipetted off and placed into a 96 well HPLC plate and spun at 10,000×G for 10 minutes to remove any cell debris. Supernatants from the previous spin were removed and placed into a fresh 96 well HPLC plate. Samples were diluted with an equal volume of 1 M ammonium acetate (pH 7) to neutralize samples for better chromatography. A 2×cGMP standard curve was prepared in 0.1 M HCl and then diluted with an equal volume of 1 M ammonium acetate, with the following final concentrations in ng/mL: 1000, 500, 250, 125, 50, 25, 5, 2.5, 0.5. The ST Core peptide (hereinafter ST Core) has the amino acid sequence: Cys Cys Glu Leu Cys Cys Asn Pro Ala Cys Thr Gly Cys Tyr (SEQ ID NO:94).

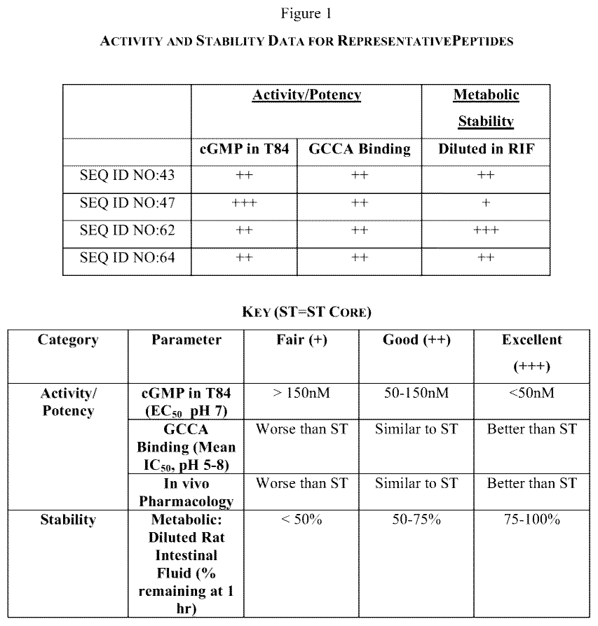

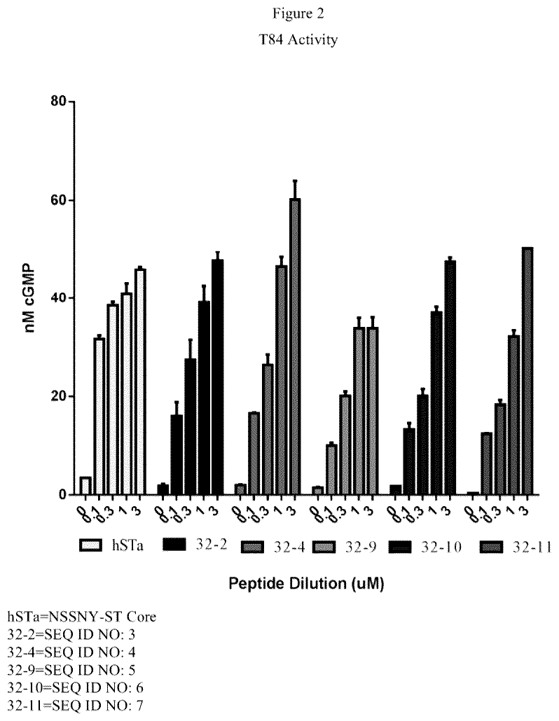

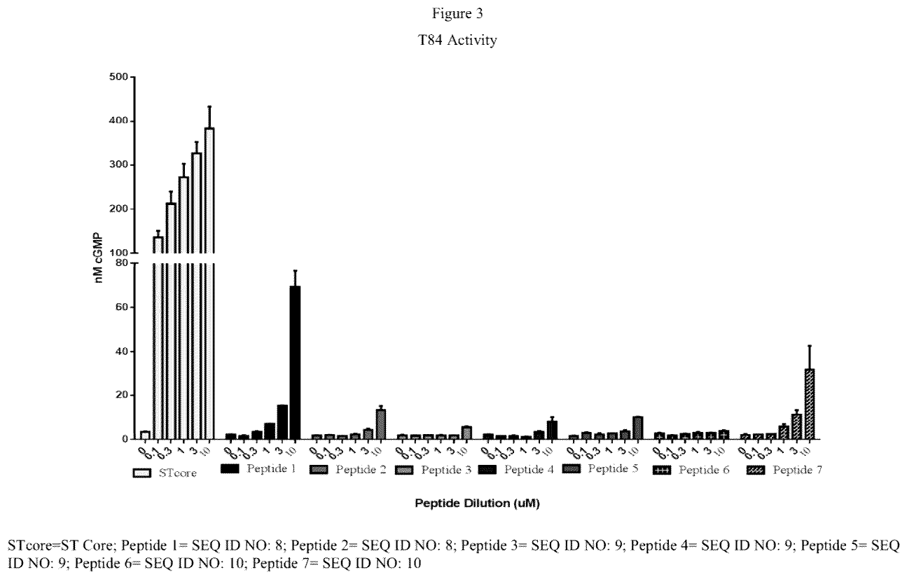

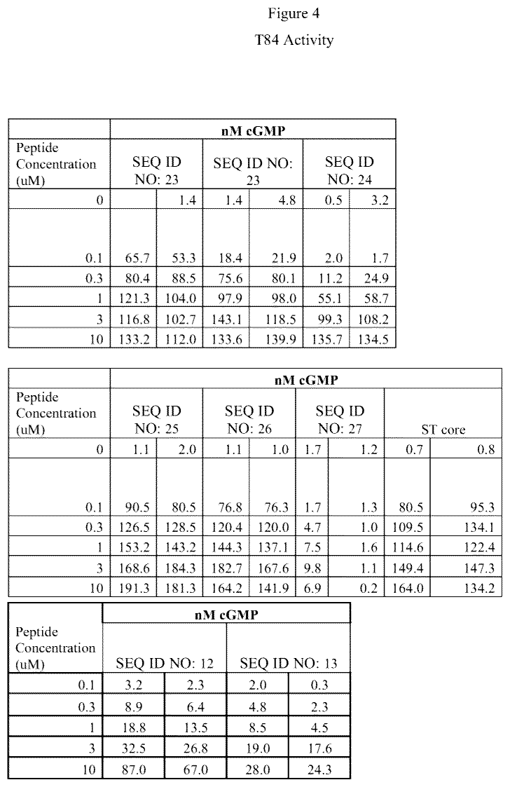

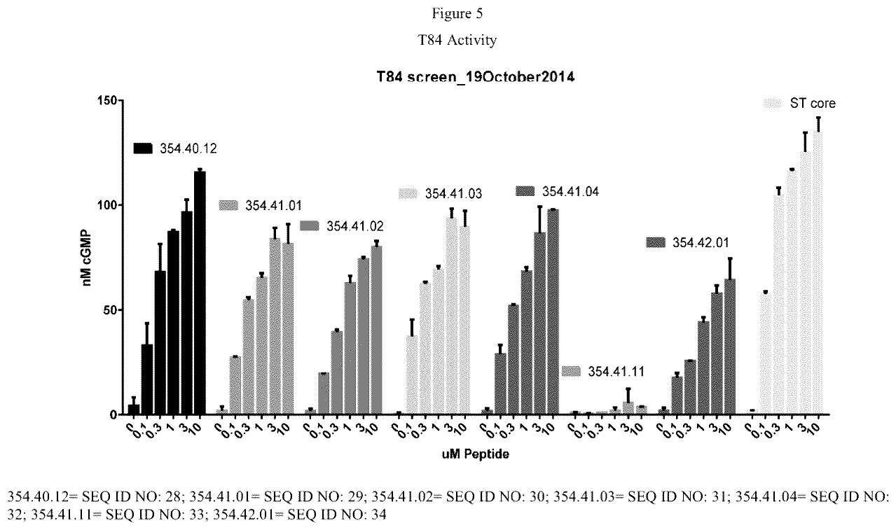

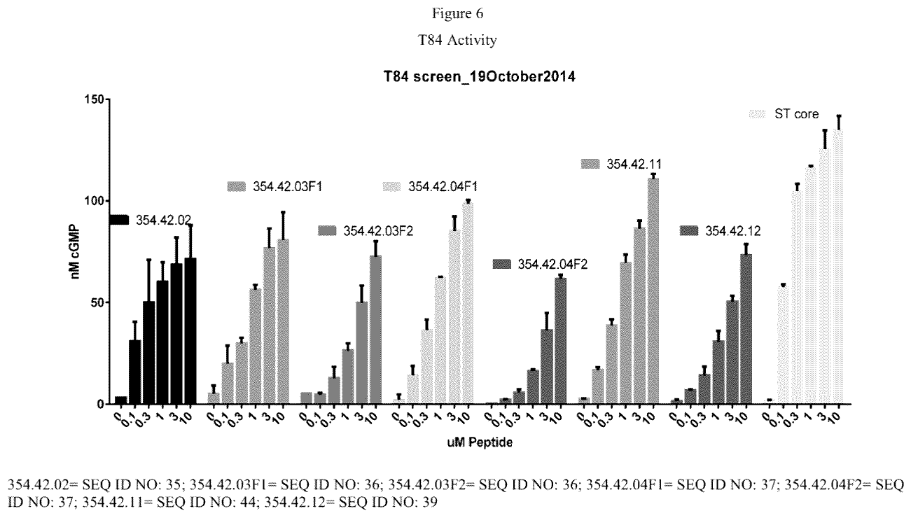

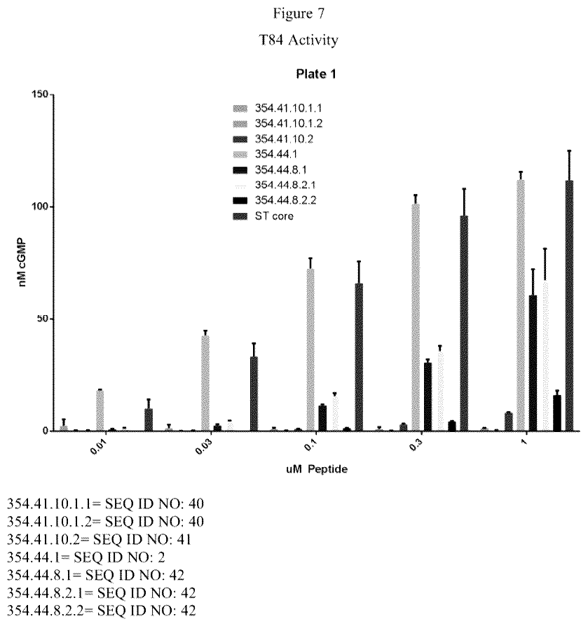

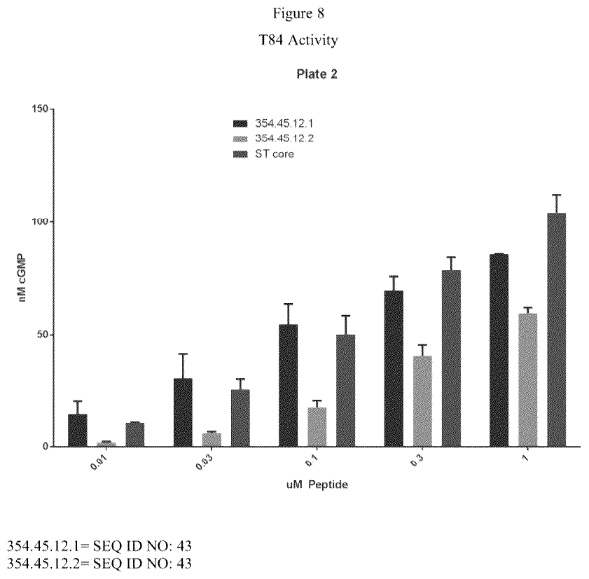

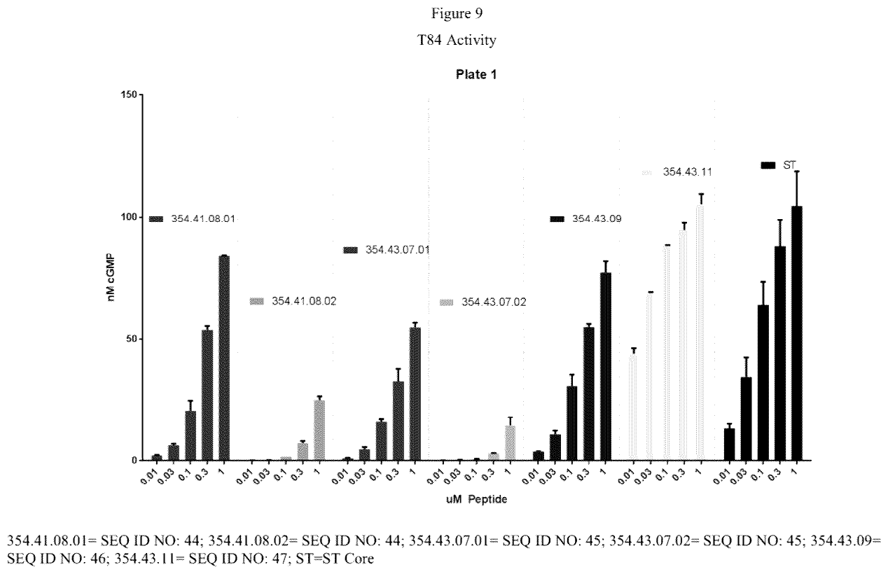

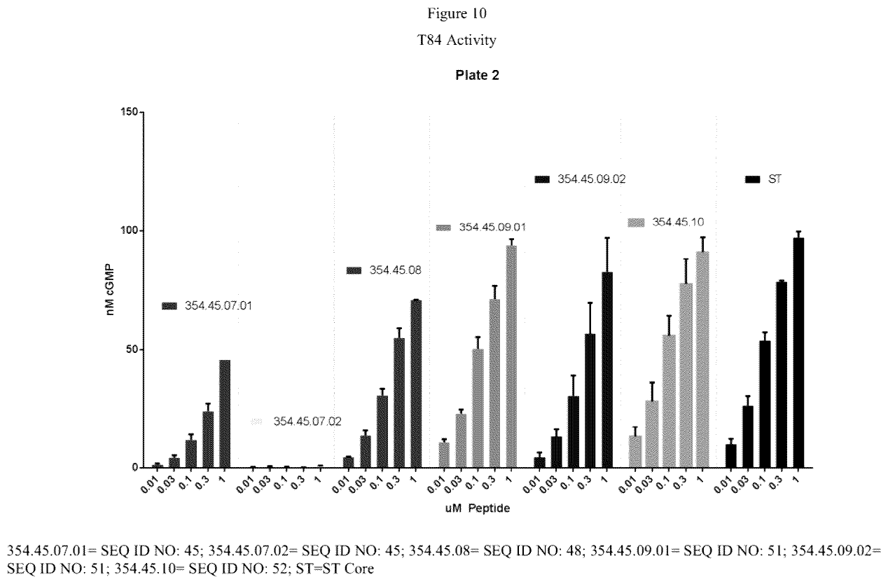

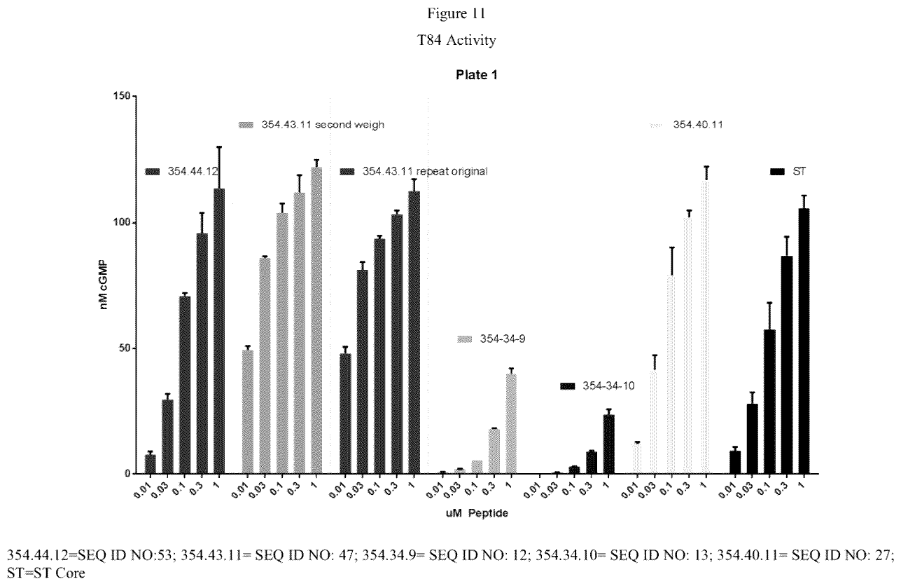

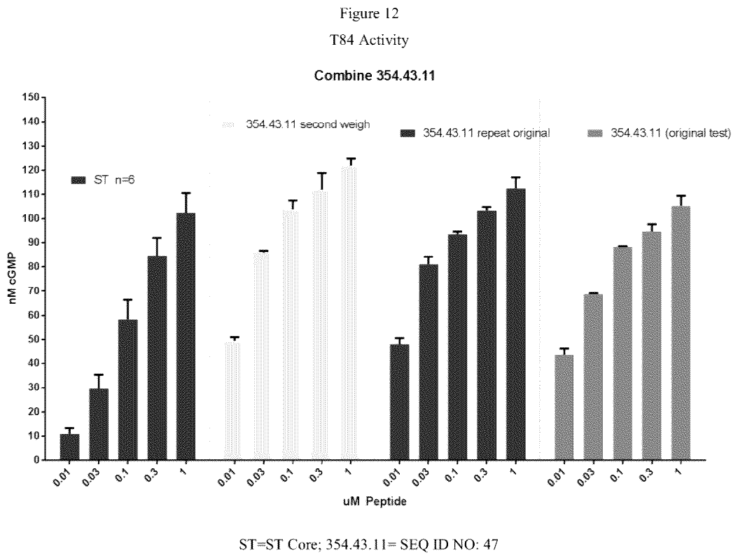

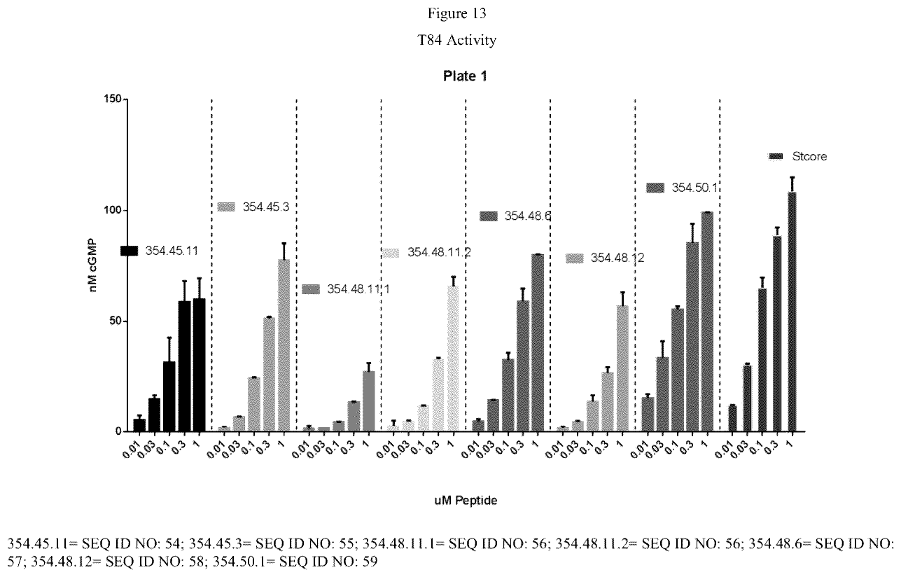

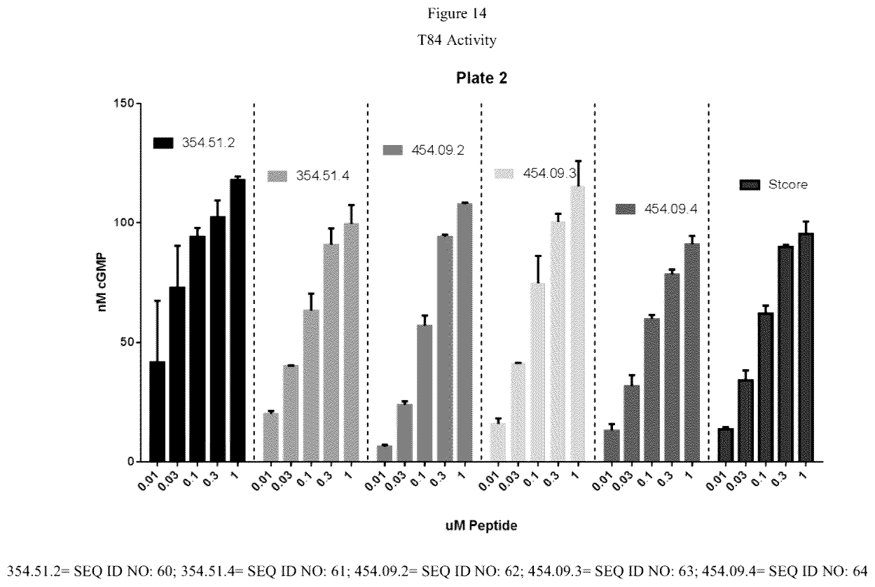

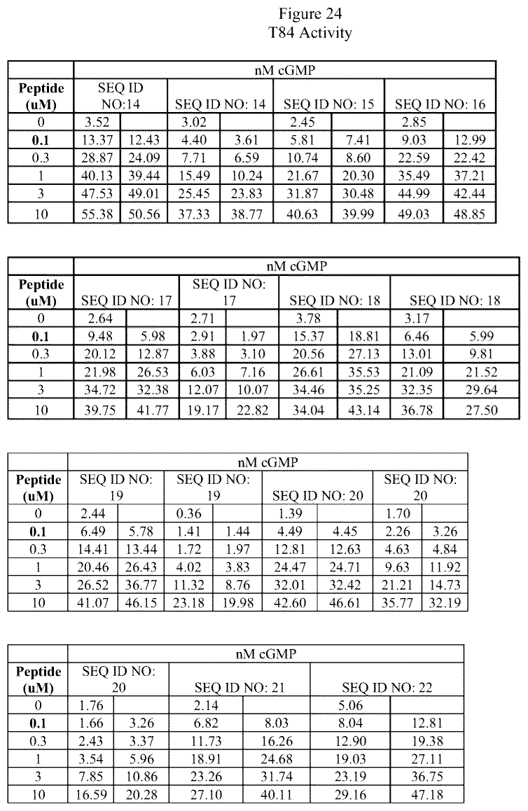

cGMP concentrations were determined from each sample using the LC/MS conditions in Table 4 and a calculated standard curve. EC₅₀ values were calculated from concentration-response curves generated with GraphPad Prism Software. Results for selected peptides may be found in FIGS. 1-14, 23 and 24.

TABLE 1
LC/MS Conditions:

| MS: | Thermo Quantum or Vantage | | | | | |
|---|---|---|---|---|---|---|
| Ion Mode: | Electropspray, positive mode (ESI⁺⁾ | | | | | |
| Scan Type: | Multiple reaction monitoring (MRM) | | | | | |
| | | Dwell | | | Retention | |
| | | Time | Collision | Tube | Time | LLOQ |
| Compound: | Transition | (msec) | Energy (V) | Lens | (min) | (ng/mL) |
| cGMP | 346 > 152 | 100 | 37 | 139 | 0.6 | 0.5 |
| (+3) cGMP | 349 > 155 | 100 | 37 | 139 | 0.6 | — |

| HPLC: | Waters Acquity UPLC | | |
|---|---|---|---|
| Column: | Hypersil Gold C18, 2.1 × 50 mm, 1.9 um | | |
| Guard Column: | Hypersil Gold, 2.1 × 10 mm, 5 um | | |
| Flow Rate: | 750 uL/min | | |
| Column Temp: | Room Temperature | | |
| Autosampler Temp: | 6° C. | | |
| Injection Volume: | 20 uL | | |
| Mobile Phases: | A = 0.1% formic acid in 100% Water | | |
| | B = 0.1% formic acid in 100% acetonitrile | | |
| Gradient: | Time (min) | % A | % B |
| | 0 | 100 | 0 |
| | 0.2 | 100 | 0 |
| | 0.3 | 50 | 50 |
| | 0.7 | 50 | 50 |
| | 0.8 | 100 | 0 |

### Example 2: GC-C Binding Assay

All GC-C binding was done in a final volume of 200 μL of media at pH 5, 7 or 8. Media at pH 5 was prepared using DMEM and 0.50% BSA and 20 mM of 2-(N-morpholino)ethanesulfonic acid (MES). Media at pH 7 was prepared using DMEM and 0.50% BSA. Media at pH 8 was prepared using DMEM and 0.500 BSA with 20 mM of sodium bicarbonate.

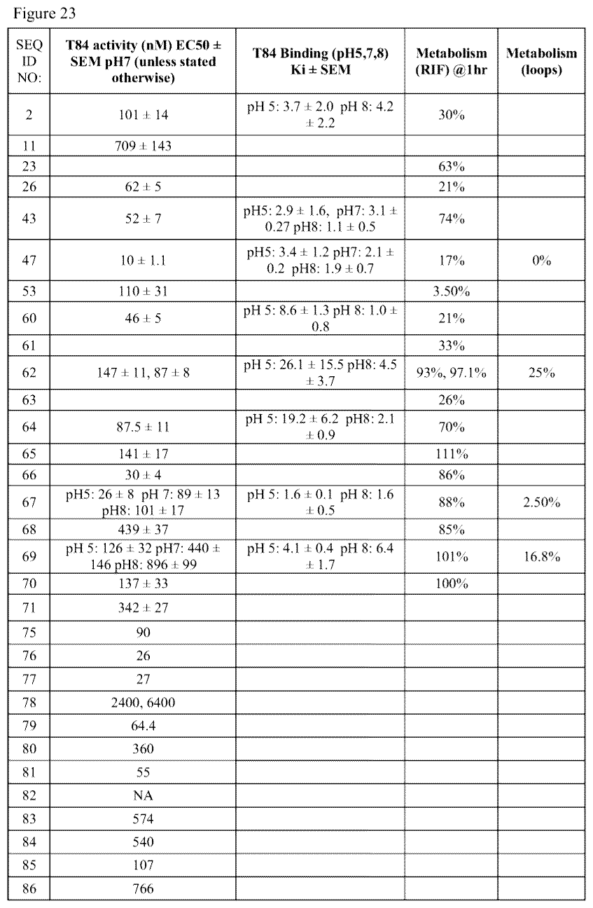

T84 cells were used at a rate of 250,000 cells per reaction. The cells were grown to confluence on T-150 flasks using DMEM-F12 50/50 media and 5 mM L-glutamine and 500 FBS. Cells were scraped off using DMEM and 0.50% BSA and counted to determine how much volume to add to give 250,000 cells per reaction in a final volume of 200 μL. Then 200,000 CPM per reaction of 1125-STp, cold peptide competitor and then T84 cells to start the reaction. Samples were then incubated at 37° C. for 1 hour. After 1 hour the entire sample was added to the pre-blocked GF-C plates and suctioned through. Then each well was washed twice with 200 μL of cold PBS. The bottom of the filter plate was removed and the 96 well plate was placed at 50 degrees to dry. After drying, 100 μL of scintillation fluid was added to each well and gently vortexed before counting. Results for selected peptides may be found in FIGS. 1 and 23.

### Example 3: Rat Intestinal Fluid (RIF) In Vitro Metabolism Incubations

Rat intestinal fluid was obtained by adding PBS to ligated ratjejunal loops for thirty minutes. The fluid was then collected, pooled and kept on ice before centrifugation at 4° C. The supernatant was removed and flash frozen. Then 60 μM of peptide (100 μg/mL) was added to rat intestinal fluid along with PBS and 0.5% BSA. The control incubations were performed in PBS. Then 50 μL aliquots from all samples were taken in duplicate at 0, 10, 30 and 60 minutes and stopped with 12% trichloroacetic acid containing internal standard.

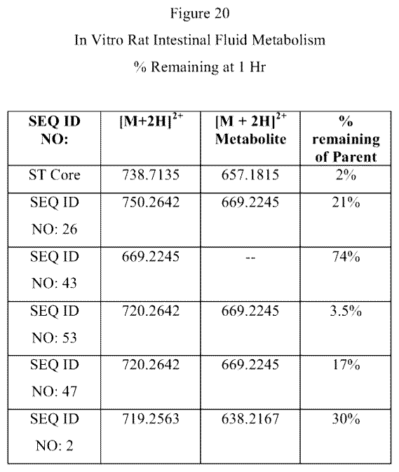

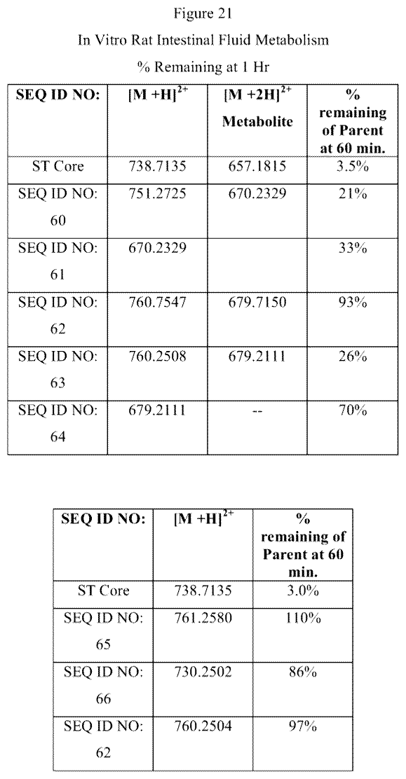

Samples were spun and the supernatant was removed for analysis by LC-MS using the calculated accurate mass of each peptide (or predicted metabolites) to generate extracted ion chromatograms. The relative response factor (analyte peak area/internal standard peak area) for each sample was used to construct a percent remaining relative to time=0. Results for selected peptides may be found in FIGS. 1, 20, 21 and 23.

### Example 4: In Vivo Ligated Rat Loops

First 60 μM of peptide was prepared in 200 μL of PBS. This solution was injected into ligated rat duodenal loops, which were approximately 3-5 cm in length. Three animals per peptide, per time point were used. At 30 minutes and 60 minutes the loops were excised, measured and weighed. Then the fluid was collected and flash frozen in Eppendorf tubes and the loops were re-weighed to determine their empty weight. Samples were then thawed and spun and the supernatant was removed from each sample. Then 50 μL aliquots were removed and added to 12% trichloroacetic acid containing an internal standard.

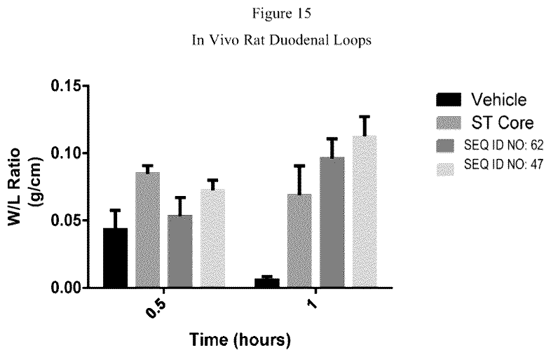

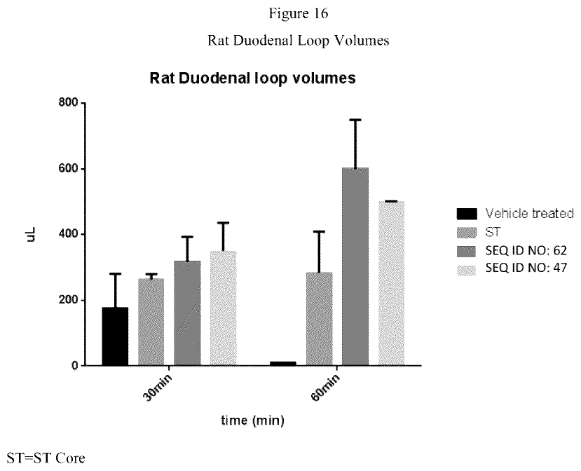

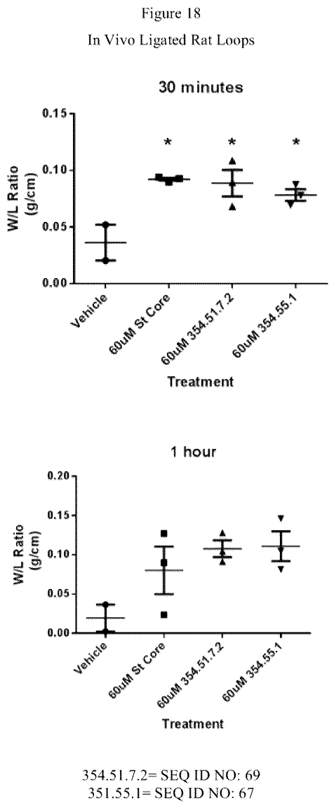

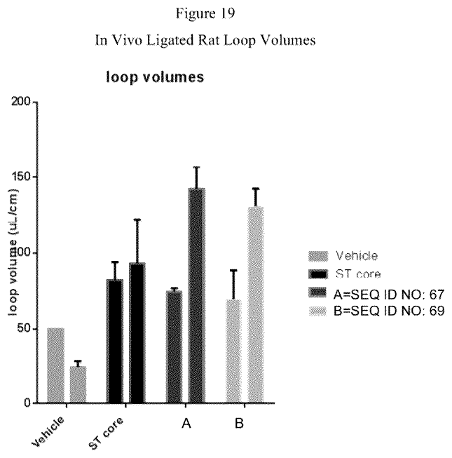

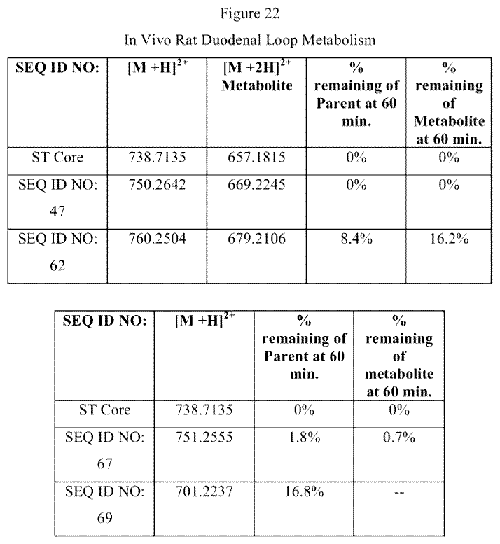

Analysis was done by LC-MS using the calculated accurate mass of each peptide (or predicted metabolites) to generate extracted ion chromatograms. The relative response factor (analyte peak area/internal standard peak area) for each sample was used to construct a percent remaining relative to time=0. The amount of fluid secretion during the incubation was determined by the formula (fluid secretion=(loop full−loop empty)/length). This formula was used to calculate how much fluid was pulled into the loops at each time point. Results for selected peptides may be found in FIGS. 15, 16, 18, 19, 22 and 23.

### Example 5: Preparation of Dicarba Peptides

Dicarba peptides were made using standard Fmoc-/Trt/Otbu protected amino acids. The dicarba peptides were synthesized on a Protein Technologies Symphony X® with the amino acids dissolved in dimethyl formamide (DMF) at a concentration of 0.5 M. The Fmoc-Cys(Trt)-OH was dissolved into a solution containing 0.5 M oxyma pure in DMF. Then HCTU was dissolved in NMP to 0.5 M, and a 1.0 M DIPEA in NMP was also used. DIC is dissolved in NMP to 0.5 M. Fmoc deprotection was performed using a solution containing 20% piperidine in DMF.

Fmoc-Cys(Trt)-O-Wang resin (0.2 mmol, 0.29 mmol/g) was treated with 20% piperidine in DMF (3×6 mL, 3 min, 1×6 mL, 10 min). The resulting resin was washed with DMF (6×10 mL, 30 s). A solution containing Fmoc-Xaa-OH, HCTU, and DIPEA in NMP that has been pre-mixed for 30 seconds was added and the mixture was agitated for 30 minutes. The resin was filtered and washed once with DMF. A second solution containing Fmoc-Xaa-OH, HCTU, and DIPEA in NMP that has been pre-mixed for 30 seconds was added and the mixture was agitated again for 30 minutes. In the case of Fmoc-Cys(Trt)-OH, the amino acid/oxyma solution was mixed with DIC in NMP for 5 min, added to the resin, and agitated for 45 min. The resin was filtered and washed once with DMF. The resulting resin was filtered and washed with DMF (6×10 mL). The material would be subjected to the aforementioned protocol and the peptide was elongated to the full sequence. To dry the resin before metathesis, the resin was washed with CH₂Cl₂ (3×10 mL) and hexanes (3×10 mL), and dried under diminished pressure overnight. The material was subjected to the aforementioned protocol and the peptide was elongated until macrocyclization.

To perform metathesis on the peptides, the following procedure was used. To 500 mg (0.1 mmol) of protected resin-bound peptide was added a 6 mg/mL solution of HGII in 5 mL of 4:1 1,2-dichloroethane-0.4M LiCl in DMA. The resulting suspension was heated in a microwave with 200 W to 160° C. for 5 minutes. The suspension was cooled, filtered and washed with dichloromethane (3×10 mL) and NMP (3×10 mL). A small portion was cleaved to determine if the metathesis was complete. The resin was soaked in a 10% DMSO in NMP solution (10 mL) overnight to scavenge excess HGII catalyst. The resulting resin was washed with NMP (3×10 mL), and treated with 20% piperidine in DMF (3×15 mL, 10 min). The resin was washed with DMF (5×10 mL), CH₂Cl₂ (3×10 mL) and hexanes (3×10 mL), and dried under diminished pressure.

To cleave the peptide from the resin, the resin was treated with a solution containing 90:5:5 TFA-TIPS-H₂O (20 mL). After 2 hours, the resin was filtered and washed with TFA (3 mL) and concentrated by 50%. Cold (−78° C.) ether was added to the solution (50 mL) and the resulting mixture was centrifuged @ 3500 rpm for 10 min. The ether was decanted and the solid was subjected to 2 additional washes and centrifuged with cold (−78° C.) ether. The resulting solid was dried under diminished pressure, dissolved into 1:1 H₂O-ACN, frozen and lyophilized.

The peptides were purified on a Waters Autopure® system using 0.10% TFA in water and 0.1% TFA in acetonitrile on a Waters PST C18 RP column (250×30 mm, 10μ, 130 Å) at a flow rate of 40 mL/min. A linear gradient was used with 5-45% acetonitrile over 40 or 60 minutes. Fractions containing the desired product were pooled and oxidized.

To oxidize the cysteine residues for disulfide bond formation, the following procedure was used. To a solution containing 100 mL of 0.05 N NH₄HCO₃ (pH ˜8) in 9:1 water-acetonitrile was added 5 mL of DMSO. After 72 hours, oxidation appeared complete by HPLC and the material was acidified with acetic acid, frozen and lyophilized. The peptides were purified on a Waters Autopure system using 0.1% TFA in Water and Acetonitrile on a Waters PST C18 RP column (250×30 mm, 10μ, 130 Å) at a flow rate of 40 mL/min. A linear gradient was used from 5-40% acetonitrile over 60 minutes. Fractions containing the desired product were pooled and lyophilized.

### Example 6: Preparation of Cystathionine Containing Peptides

The material was synthesized on a Protein Technologies Symphony X® with amino acids dissolved in DMF at 0.5M. Fmoc-Cys(Trt)-OH was dissolved into a solution containing 0.5 M oxyma pure in DMF. HCTU was dissolved in NMP to 0.5 M, and a 1.0 M DIPEA in NMP was also used. DIC was dissolved in NMP to 0.5 M. Fmoc-deprotection was performed using a solution containing 20% piperidine in DMF.

To couple the peptide to the resin, Rink amide resin (0.2 mmol, 0.24 mmol/g) was treated with 20% piperidine in DMF (3×6 mL, 3 min, 1×6 mL, 10 min). The resulting resin was washed with DMF (6×10 mL, 30 s). A solution containing Fmoc-Xaa-OH, HCTU, and DIPEA in NMP that has been pre-mixed for 30 seconds was added and the mixture was agitated for 30 minutes. The resin was filtered and washed once with NMP. A second solution containing Fmoc-Xaa-OH, HCTU, and DIPEA in NMP that has been pre-mixed for 30 seconds was added and the mixture was agitated again for 30 minutes. In the case of Fmoc-Cys(Trt)-OH, the amino acid/oxyma pure solution was mixed with DIC in NMP for 5 minutes, added to the resin, and agitated for 45 min. The resin was filtered and washed once with DMF. The resulting resin was filtered and washed with DMF (6×10 mL). The material was subjected to the aforementioned protocol and the peptide was elongated until macrocyclization.

To couple the diamino acid, the resin was treated with 20% piperidine in DMF (3×6 mL, 3 min, 1×6 mL, 10 min). The resulting resin was washed with DMF (6×10 mL, 30 s). A solution containing alloc-HCys((Fmoc-Ala-OH)-3-yl)-all (227 mg, 0.4 mmol), PyAOP (209 mg 0.4 mmol) and DIPEA (139 μl, 125 mg, 0.8 mmol) in 5 mL of NMP was added to the resin. After 90 min, the resin was filtered and washed with DMF (6×10 mL). The peptide was then elongated using the aforementioned protocol.

To perform Allyl-Alloc deprotection, the resin (0.2 mmol) was suspended in 10 mL of DMF and a solution containing Pd(PPh3)4 (300 mg, 0.26 mmol) in 10 mL of CH2Cl2 was added followed by 0.25 mL (2 mmol) of phenyl silane. The resulting mixture was shaken in the absence of light for 2 hours. A small sample was cleaved to ensure complete deprotection. The resulting resin was filtered and washed with CH2Cl2 (3×10 mL) and DMF (3×10 mL). The resin was treated with a solution containing 0.5% sodium diethyldithiocarbamate in DMF (10 mL, 4×15 min), and washed with DMF (3×10 mL).

To perform macrocyclization, the resin was treated with a 20% piperidine in DMF solution (2×5 min, 1×10 min, 15 mL) and washed with DMF (6×15 mL). A solution containing 521 mg (1 mmol) of PyAOP in 15 mL of DMF was added and after 1 minute, 0.35 mL (2 mmol) of DIPEA was added and shaken for 60 minutes. A small sample was taken for analysis. The resulting resin was washed with DMF (3×15 mL) and placed back onto the SymphonyX to complete the synthesis.

To cleave the peptide from the resin, the resin was treated with a solution containing 90:5:3:2 TFA-TIPS-DODT-H₂O (20 mL). After 2 hours, the resin was filtered and washed with TFA (3 mL) and concentrated by 50%. Cold (−78° C.) ether was added to the solution (50 mL) and the resulting mixture was centrifuged @ 3500 rpm for 10 minutes. The ether was decanted and the solid was subject to 2 additional washes and centrifuged with cold (−78° C.) ether. The resulting solid was dried under diminished pressure, dissolved into 1:1 H2O-ACN, frozen and lyophilized. The peptides were then purified on a Waters autopure system using 0.1% TFA in water and 0.1% TFA in acetonitrile on a Waters PST C18 RP column (250×30 mm, 10μ, 130 Å) at a flow rate of 40 mL/min. A linear gradient was used 5-45% acetonitrile over 40 or 60 minutes. Fractions containing the desired product were pooled and oxidized.

Oxidation was performed adding 5 mL of DMSO to a solution containing 100 mL of 0.05 N NH₄HCO₃ (pH ˜8) in 9:1 water-acetonitrile. After 72 hours, oxidation appeared complete by HPLC and the material was acidified with acetic acid, frozen and lyophilized. The peptides were purified on a Waters Autopure® system using 0.1% TFA in water and 0.1% TFA in acetonitrile on a Waters PST C18 RP column (250×30 mm, 10μ, 130 Å) at a flow rate of 40 mL/min. A linear gradient was used from 5-40% acetonitrile over 60 minutes. Fractions containing the desired product were pooled and lyophilized.

### Example 7. Production of Lactam Bond Containing Peptides

Peptides were synthesized using standard Fmoc-/Trt/Otbu protected amino acids. Material was synthesized on a Protein Technologies Symphony X® with amino acids dissolved in DMF at 0.5M. Fmoc-Cys(Trt)-OH was dissolved into a solution containing 0.5 M oxyma pure in DMF. HCTU was dissolved NMP to 0.5 M, and a 1.0 M DIEA in NMP was also used. DIC was dissolved in NMP to 0.5 M. Fmoc-deprotection was performed using a solution containing 20% piperidine in DMF. Uncommon amino acids were coupled manually as described below.

To incorporate Fmoc-Dpr(ivDde)-OH, a solution containing 533 mg (1.0 mmol) of Fmoc-Dpr(ivDde)-OH, 521 mg (1.0 mmol) of PyAOP and 348 μL (258 mg, 2.0 mmol) of DIPEA in 6 mL of DMF was added to the deprotected resin (0.2 mmol). After 90 min, the resin was filtered and washed with DMF (3×10 mL), CH₂Cl₂ (3×10 mL) and again with DMF (3×10 mL). The resin was placed back onto the synthesizer to continue the synthesis.

To incorporate Fmoc-Asp(ODmab)-OH, a solution containing 667 mg (1.0 mmol) of Fmoc-Asp(Odmab)-OH, 521 mg (1.0 mmol) of PyAOP and 348 μL (258 mg, 2.0 mmol) of DIPEA in 6 mL of DMF was added to the deprotected resin (0.2 mmol). After 90 min, the resin was filtered and washed with DMF (3×10 mL), CH₂Cl₂ (3×10 mL) and again with DMF (3×10 mL). The resin was placed back onto the synthesizer for final deprotection.

To Boc protect the N-terminus, the resin was treated with 218 mg (1.0 mmol) di-tert-butyl dicarbonate in 5 mL of DMF. After 4 h, a small sample (−10 mg) was removed and acetylated with 10 μL of acetic anhydride and 30 μL of DIPEA in DMF to check for completion. The resulting resin was washed with DMF (5×10 mL) and treated with 100 μL of acetic anhydride and 300 μL of DIPEA in DMF for 50 min. the resulting resin was washed with DMF (6×10 mL)

To remove ivDDE and Dmab, the protected resin was treated with a solution containing 2% hydrazine monohydrate in DMF (5×5 mL, 5 min). The resulting resin was washed with DMF (6×10 ml).

To form the lactam, the resin was treated with 521 mg (1.0 mmol) of PyAOP and 348 μL (258 mg, 2.0 mmol) of DIPEA in 5 mL of DMF. The resulting mixture was heated via microwave heating (200 W) to 100° C. for 10 min. The resulting resin was cooled, filtered and washed with DMF (6×10 mL) and CH₂Cl₂ (6×10 mL) and dried under diminished pressure.

To cleave the peptide from the resin, the resin was treated with a solution containing 90:5:3:2 TFA-TIPS-DODT-H2O (20 mL). After 2 h, the resin was filtered and washed with TFA (3 mL) and concentrated by 50%. Cold (−78° C.) either was added to the solution (50 mL) and the resulting mixture was centrifuged @ 3500 rpm for 10 min. The ether was decanted and the solid was subject to 2 addition washes/centrifuge with cold (−78° C.) ether. The resulting solid was dried under diminished pressure, dissolved into 1:1 H2O-ACN, frozen and lyophilized.

To oxidize the peptide, the crude peptide (280 mg) was dissolved in water and the mixture was treated with solid NH₄HCO₃ to adjust the pH to be >7. After which 20 mL of acetonitrile was added followed by 10 mL of DMSO. After 24 h, oxidation appeared complete by HPLC and the material was acidified to pH ˜2 with trifluoroacetic acid, filtered, frozen and lyophilized.

The peptide was purified on a Waters Autopure® system using 0.1% TFA in water and 0.1% TFA in acetonitrile was used on a Waters PST C18 RP column (250×30 mm, 5μ, 130 Å) at a flow rate of 40 mL/min. A linear gradient from 5% 0.1% TFA in acetonitrile to 25% 0.1% TFA in acetonitrile over 40 min was used Fractions containing desired product were pooled, frozen and lyophilized. The material was subjected to a second pass purification.

The peptide was purified on a Waters Autopure® system using 0.1% TFA in water and 0.1% TFA in acetonitrile was used on a Waters PST C18 RP column (250×19 mm, 5μ, 130 Å) at a flow rate of 20 mL/min. A linear gradient from 15% 0.1% TFA in acetonitrile to 25% 0.1% TFA in acetonitrile over 60 min was used. Fractions containing desired product were pooled, frozen and lyophilized to give a colorless solid.

### Example 8: Gastrointestinal Transit in Mice

The purpose of the assay was to test the effect of the guanylate cyclase C agonist peptides on in vivo gastrointestinal transit in mice. Orally-dosed guanylate cyclase C agonists have been demonstrated to increase the % Distance Travelled by a charcoal meal in mice.

For the assay, female CD-1 mice (n=10 per group) weighing 25-30 g were fasted overnight and given access to water ad libitum. Activated charcoal (20 g; 100 mesh; Sigma cat #242276) was suspended in 200 mL gum arabic (100 mg/mL), and stirred for at least one hour. Test peptides were prepared in a 20 mM Tris pH 6.9 vehicle.

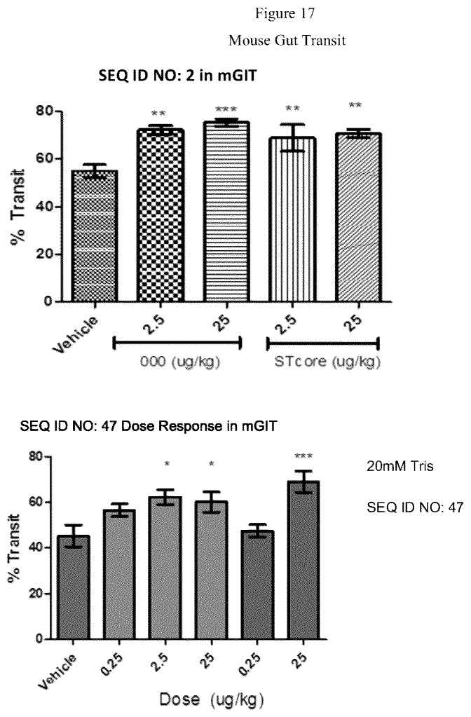

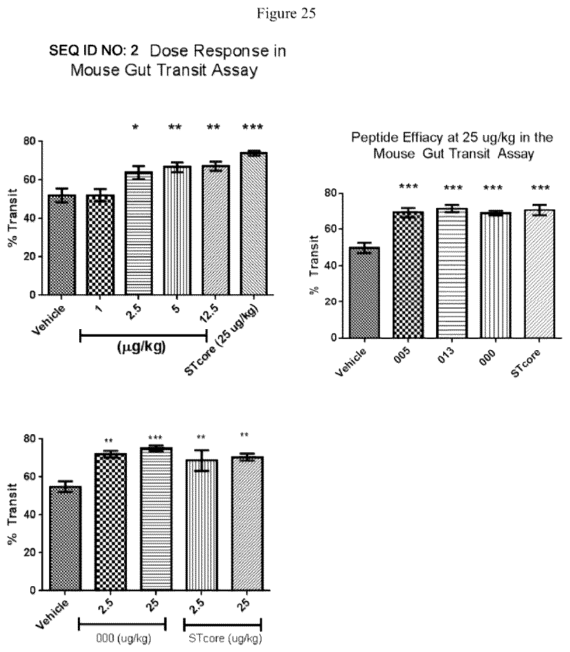

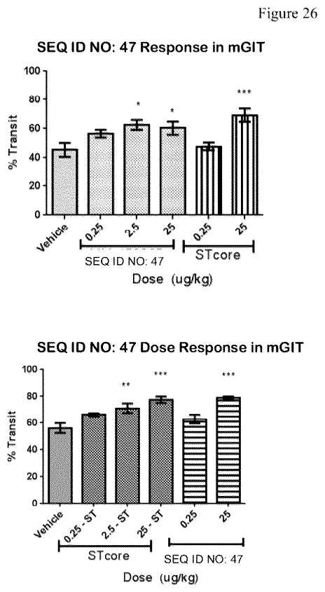

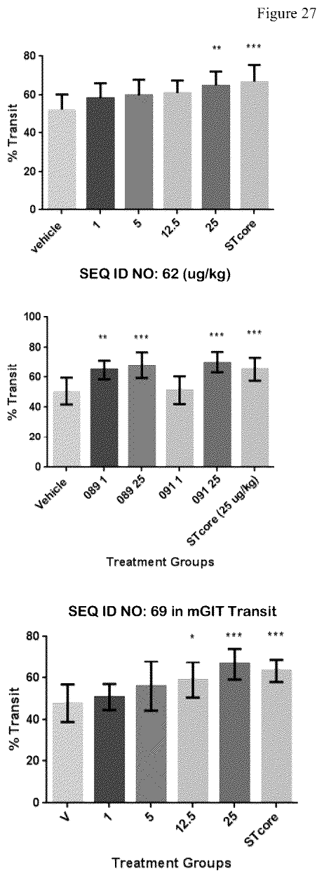

Test peptide and vehicle were administered in 200 μL doses by oral gavage. Seven minutes after dosing the test peptides, 200 μL of the charcoal/gum arabic suspension was dosed by oral gavage. After 15 minutes, mice were sacrificed by CO₂ overdose. The gastrointestinal tract was removed from the esophagus to the caecum. The total length of the small intestine was measured from the pyloric junction to the ileocaecal junction. The distance travelled by the charcoal was measured from the pyloric junction to the charcoal front. The Distance Travelled (%) was determined as (distance travelled by charcoal/total length of the small intestine)×100. Data were entered into the GraphPad Prism software program and analyzed by ANOVA using a Bonferroni multiple comparison test post-hoc. Plots of data and ED₅₀ were also determined using the GraphPad Prism software package. Results for selected peptides may be found in FIGS. 17, 25, 26, and 27.

### Other Embodiments

All publications and patents referred to in this disclosure are incorporated herein by reference to the same extent as if each individual publication or patent application were specifically and individually indicated to be incorporated by reference. Should the meaning of the terms in any of the patents or publications incorporated by reference conflict with the meaning of the terms used in this disclosure, the meaning of the terms in this disclosure are intended to be controlling. Furthermore, the foregoing discussion discloses and describes merely exemplary embodiments of the present invention. One skilled in the art will readily recognize from such discussion and from the accompanying drawings and claims, that various changes, modifications and variations can be made therein without departing from the spirit and scope of the invention as defined in the following claims.

What is claimed is:

1. A peptide or a pharmaceutically acceptable salt thereof, wherein the peptide comprises the amino acid sequence
Xaa₁ Xaa₂ Xaa₃ Xaa₄ Xaa₅ Xaa₆ Xaa₇ Xaa₈ Xaa₉ Xaa₁₀ Xaa₁₁ Xaa₁₂ Xaa₁₃ Xaa₁₄ Ala₁₅ Xaa₁₆ Xaa₁₇ Gly₁₈ Xaa₁₉ Xaa₂₀ Xaa₂₁ (SEQ ID NO: 1), or a pharmaceutically acceptable salt thereof; wherein
Xaa₁ is BE or is absent;
Xaa₂ is BK, Asn or is absent;
Xaa₃ is Asn, Ser or is absent;
Xaa₄ is Ser or is absent;
Xaa₅ is Ser, Asn, Ile, BE or is absent;
Xaa₆ is Tyr, Asp, 4-fluorophenylalanine ((4-F)Phe), BK or is absent;
Xaa₇ is Cys, cystathionine (Cth), allylglycine (Ag), Hag, or Asp;
Xaa₈ is Cys, cystathionine (Cth), penicillamine (Pen), or allylglycine (Ag);
Xaa₉ is Glu, Asp, Ser, Thr, or Gln;
Xaa₁₀ is Leu, cyclohexylalanine (Cha), Phe, or 4-fluorophenylalanine ((4-F)Phe);
Xaa₁₁ is Cys, Ag, or penicillamine (Pen);
Xaa₁₂ is Cys, allylglycine (Ag), Hag, Cth, Dpr, or Val;
Xaa₁₃ is Asn or Leu;
Xaa₁₄ is Pro, Val, sarcosine (Sar), Leu, or Hydroxyproline (OH-Pro);
Xaa₁₆ is Cys, Ag, Pen or Cth;
Xaa₁₇ is Tyr, Thr, cyclohexylalanine (Cha), 4-fluorophenylalanine ((4-F)Phe), Phe, Ser, or Ala;
Xaa₁₉ is Cys, Ag or Pen;
Xaa₂₀ is Tyr, Leu, 4-fluorophenylalanine ((4-F)Phe), cyclohexylalanine (Cha), D-Tyr, N-Methyl Tyr (Nme-Tyr) or is absent;
Xaa₂₁ is absent or Asn;
wherein at least one Xaa is BE, BK, (4-F)Phe, Cth, Ag, Hag, Pen, Cha, Sar, Dpr, or OH-Pro; and
wherein the peptide contains a covalent bond between Xaa₇ and Xaa₁₂, Xaa₈ and Xaa₁₆ and Xaa₁₁ and Xaa₁₉.

2. The peptide or pharmaceutically acceptable salt thereof according to claim 1, wherein the N-terminus is capped with acetic acid, pentenoic acid, biotin, 4-Mepip, C12 alkyl carboxylic acid, C14 alkyl carboxylic acid, C16 alkyl carboxylic acid, or C18 alkyl carboxylic acid.

3. The peptide or pharmaceutically acceptable salt thereof according to any one of claims 1 or 2, wherein the C-terminus is amidated.

4. The peptide or pharmaceutically acceptable salt thereof according to any one of claims 1-3, wherein
Xaa₇ and Xaa₁₂ are both Ag and a dicarba bond is present between Ag₇ and Ag₁₂;
Xaa₇ and Xaa₁₂ are both Cys and a disulfide bond is present between Cys₇ and Cys₁₂;
Xaa₈ and Xaa₁₆ are both Cys and a disulfide bond is present between Cys₈ and Cys₁₆;
Xaa₇ is Cth and Xaa₁₂ is Cys and a bond is present between Cth₇ and Cys₁₂;
Xaa₈ is Cth and Xaa₁₆ is Cys and a bond is present between Cth₈ and Cys₁₆;
Xaa₁₁ and Xaa₁₉ are Cys and a disulfide bond is present between Cys₁₁ and Cys₁₉; or
any combination thereof.

5. The peptide or pharmaceutically acceptable salt thereof according to any one of claims 1-3, wherein Xaa₇ is allylglycine, Cth or Cys.

6. The peptide or pharmaceutically acceptable salt thereof according to any one of claims 1-3, wherein Xaa₈ is Cys or cystathionine.

7. The peptide or pharmaceutically acceptable salt thereof according to any one of claims 1-6, wherein Xaa₉ is Glu.

8. The peptide or pharmaceutically acceptable salt thereof according to any one of claims 1-7, wherein Xaa₁₀ is Leu.

9. The peptide or pharmaceutically acceptable salt thereof according to any one of claims 1-8, wherein Xaa₁₂ is Cys or allylglycine.

10. The peptide or pharmaceutically acceptable salt thereof according to any one of claims 1-9, wherein Xaa₁₄ is Val or Pro.

11. The peptide or pharmaceutically acceptable salt thereof according to any one of claims 1-10, wherein Xaa₁₇ is Tyr or Thr.

12. The peptide or pharmaceutically acceptable salt thereof according to any one of claims 1-11, wherein Xaa₂₀ is Tyr or is absent.

13. The peptide or pharmaceutically acceptable salt thereof according to any one of claims 1-12, wherein Xaa₂₁ is absent.

14. The peptide or pharmaceutically acceptable salt thereof according to claim 1, wherein:
Xaa₁ is absent;
Xaa₂ is absent;
Xaa₃ is absent;
Xaa₄ is absent;
Xaa₅ is absent;
Xaa₆ is absent;
Xaa₇ is Ag, Cys, or Cth;
Xaa₈ is Cys or Cth;
Xaa₉ is Glu;
Xaa₁₀ is Leu;
Xaa₁₂ is Ag or Cys;
Xaa₁₄ is Val or Pro;
Xaa₁₇ is Tyr or Thr; and
Xaa₂₀ is Tyr or is absent.

15. The peptide or pharmaceutically acceptable salt thereof according to claim 13, wherein the peptide comprises the amino acid sequence:
(SEQ ID NO: 2)
H-Ag Cys Glu Leu Cys Ag Asn Pro Ala Cys Thr Gly Cys Tyr-NH₂;
(SEQ ID NO: 26)
H-Ag Cys Glu Leu Cys Ag Asn Pro Ala Cys Tyr Gly Cys Tyr-NH₂;
(SEQ ID NO: 43)
H-Ag Cys Glu Leu Cys Ag Asn Pro Ala Cys Tyr Gly Cys-COOH;
(SEQ ID NO: 47)
H-Ag Cys Glu Leu Cys Ag Asn Val Ala Cys Thr Gly Cys Tyr-NH₂;
(SEQ ID NO: 62)
H-Ag Cys Glu Leu Cys Ag Asn Val Ala Cys Tyr Gly Cys Tyr-NH₂;
(SEQ ID NO: 64)
H-Cth Cys Glu Leu Cys Cys Asn Pro Ala Cys Tyr Gly Cys-COOH;
(SEQ ID NO: 68)
Ac-Cys Cth Glu Leu Cys Cys Asn Pro Ala Cys Tyr Gly Cys Tyr-NH₂;
(SEQ ID NO: 69)
Ac-Cys Cth Glu Leu Cys Cys Asn Val Ala Cys Tyr Gly Cys-COOH;
(SEQ ID NO: 70)
H-Cys Cth Glu Leu Cys Cys Asn Val Ala Cys Tyr Gly Cys-COOH;
or
(SEQ ID NO: 71)
Ac-Cys Cth Glu Leu Cys Cys Asn Pro Ala Cys Tyr Gly Cys-COOH.

16. The peptide or a pharmaceutically acceptable salt thereof according to claim 1, wherein the peptide comprises the amino acid sequence:
(SEQ ID NO: 2)
H-Ag Cys Glu Leu Cys Ag Asn Pro Ala Cys Thr Gly Cys Tyr-NH₂;
(SEQ ID NO: 3)
C12-BE BK Asn Ser Ser Tyr Cys Cys Glu Leu Cys Cys Asn Pro Ala Cys
Thr Gly Cys Tyr-NH₂;
(SEQ ID NO: 4)
C16-BE BK Asn Ser Ser Tyr Cys Cys Glu Leu Cys Cys Asn Pro Ala Cys
Thr Gly Cys Tyr-NH₂;
(SEQ ID NO: 5)
C12-BE BK Asn Ser Ser Tyr Cys Cys Glu Leu Cys Cys Asn Pro Ala Cys
Thr Gly Cys Tyr-COOH;
(SEQ ID NO: 6)
C14-BE BK Asn Ser Ser Tyr Cys Cys Glu Leu Cys Cys Asn Pro Ala Cys
Thr Gly Cys Tyr-COOH;
(SEQ ID NO: 7)
C16-BE BK Asn Ser Ser Tyr Cys Cys Glu Leu Cys Cys Asn Pro Ala Cys
Thr Gly Cys Tyr-COOH;
(SEQ ID NO: 8)
H-Cys Ag Glu Leu Cys Cys Asn Pro Ala Ag Thr Gly Cys Tyr-NH₂;
(SEQ ID NO: 9)
H-Asn Asp Asp Ag Glu Leu Cys Val Asn Val Ala Ag Thr Gly Cys Leu-
NH₂;
(SEQ ID NO: 10)
H-Asn Asp Asp Cys Glu Leu Ag Val Asn Val Ala Cys Thr Gly Ag Leu-
NH₂;
(SEQ ID NO: 11)
C18-BE BK Asn Ser Ser Tyr Cys Cys Glu Leu Cys Cys Asn Pro Ala Cys
Thr Gly Cys Tyr-COOH;
(SEQ ID NO: 12)
H-Ag Cys Glu Leu Cys Ag Asn Pro Ala Cys Thr Gly Cys-NH₂;
(SEQ ID NO: 13)
H-Ag Cys Glu Leu Cys Ag Asn Pro Ala Cys Ala Gly Cys-NH₂;
(SEQ ID NO: 14)
H-Ag Cys Glu Phe Cys Ag Asn Pro Ala Cys Thr Gly Cys Tyr-NH₂;
(SEQ ID NO: 15)
H-Ag Cys Glu (4-F)Phe Cys Ag Asn Pro Ala Cys Thr Gly Cys-NH₂;
(SEQ ID NO: 16)
H-Ag Cys Glu (4-F)Phe Cys Ag Asn Pro Ala Cys Thr Gly Cys Tyr-NH₂;
(SEQ ID NO: 17)
H-Ag Cys Glu (4-F)Phe Cys Ag Asn Pro Ala Cys Thr Gly Cys (4-F)Phe-
NH₂;
(SEQ ID NO: 18)
H-Cys Cys Glu Leu Ag Cys Asn Pro Ala Cys Thr Gly Ag Tyr-NH₂;
(SEQ ID NO: 19)
H-Ag Cys Glu Phe Cys Ag Asn Pro Ala Cys Thr Gly Cys-NH₂;
(SEQ ID NO: 20)
H-Ag Cys Glu Phe Cys Ag Asn Pro Ala Cys Thr Gly Cys (4-F)Phe-NH₂;
(SEQ ID NO: 21)
H-Cys Cys Glu (4-F)Phe Cys Cys Asn Pro Ala Cys Thr Gly Cys Tyr-NH₂;
(SEQ ID NO: 22)
H-Cys Cys Glu (4-F)Phe Cys Cys Asn Pro Ala Cys Thr Gly Cys (4-F)Phe-
NH₂;
(SEQ ID NO: 23)
H-Ag Cys Glu Leu Cys Ag Asn Pro Ala Cys Thr Gly Cys (4-F)Phe-NH₂;
(SEQ ID NO: 24)
H-(4-F)Phe Ag Cys Glu Leu Cys Ag Asn Pro Ala Cys Thr Gly Cys (4-
F)Phe-NH₂;
(SEQ ID NO: 25)
H-Ag Cys Glu Leu Cys Ag Asn Pro Ala Cys Ala Gly Cys Tyr-NH₂;
(SEQ ID NO: 26)
H-Ag Cys Glu Leu Cys Ag Asn Pro Ala Cys Tyr Gly Cys Tyr-NH₂;
(SEQ ID NO: 27)
H-Ag Cys Glu Leu Cys Ag Asn Pro Ala Cys Phe Gly Cys Tyr-NH₂;
(SEQ ID NO: 28)
H-Ag Cys Glu Leu Cys Ag Asn Pro Ala Cys (4-F)Phe Gly Cys Tyr-NH₂;
(SEQ ID NO: 29)
H-Ag Cys Glu Leu Cys Ag Asn Pro Ala Cys Thr Gly Cys Leu-NH₂;
(SEQ ID NO: 30)
H-Ag Cys Glu Leu Cys Ag Asn Pro Ala Cys Thr Gly Cys Cha-NH₂;
(SEQ ID NO: 31)
H-Ag Cys Glu Cha Cys Ag Asn Pro Ala Cys Thr Gly Cys Tyr-NH₂;
(SEQ ID NO: 32)
H-Ag Cys Glu Leu Cys Ag Asn Pro Ala Cys Thr Gly Cys D-Tyr-NH₂;
(SEQ ID NO: 33)
H-Ag Pen Glu Leu Cys Ag Asn Pro Ala Pen Thr Gly Cys Tyr-NH₂;
(SEQ ID NO: 34)
H-Ag Cys Glu Leu Cys Ag Asn Pro Ala Cys Thr Gly Cys Leu Asn-NH₂;
(SEQ ID NO: 35)
H-Ag Cys Glu Leu Cys Ag Asn Pro Ala Cys Thr Gly Cys Tyr Asn-NH₂;
(SEQ ID NO: 36)
H-Asp Ag Cys Glu Leu Cys Ag Asn Pro Ala Cys Thr Gly Cys Tyr-NH₂;
(SEQ ID NO: 37)
H-Ile Asp Ag Cys Glu Leu Cys Ag Asn Pro Ala Cys Thr Gly Cys Tyr-
NH₂;
(SEQ ID NO: 38)
H-Ag Cys Glu Leu Cys Ag Asn OH-Pro Ala Cys Thr Gly Cys Tyr-NH₂;
(SEQ ID NO: 39)
H-Ag Cys Glu Leu Cys Ag Asn OH-Pro Ala Cys Thr Gly Cys-NH₂;
(SEQ ID NO: 40)
H-Ag Cys Glu Leu Pen Ag Asn Pro Ala Cys Thr Gly Pen Tyr-NH₂;
(SEQ ID NO: 41)
H-Ag Cys Glu Leu Pen Ag Asn Pro Ala Cys Thr Gly Pen Tyr-NH₂;
(SEQ ID NO: 42)
H-Ag Cys Glu Leu Cys Ag Asn Pro Ala Cys Tyr Gly Cys Nme-Tyr-NH₂;
(SEQ ID NO: 43)
H-Ag Cys Glu Leu Cys Ag Asn Pro Ala Cys Tyr Gly Cys-COOH;
(SEQ ID NO: 44)
H-Ag Cys Glu Leu Cys Ag Asn Pro Ala Cys Cha Gly Cys Tyr-NH₂;
(SEQ ID NO: 45)
H-Ag Cys Glu Leu Cys Ag Asn Leu Ala Cys Thr Gly Cys Tyr-NH₂;
(SEQ ID NO: 46)
H-Ag Cys Glu Leu Cys Ag Asn Sar Ala Cys Thr Gly Cys Tyr-NH₂;
(SEQ ID NO: 47)
H-Ag Cys Glu Leu Cys Ag Asn Val Ala Cys Thr Gly Cys Tyr-NH₂;
(SEQ ID NO: 48)
H-Ag Cys Glu Leu Cys Ag Asn Pro Ala Cys Tyr Gly Cys Nme-Tyr-NH₂;
(SEQ ID NO: 49)
H-Ag Cys Glu Leu Cys Ag Leu Pro Ala Cys Tyr Gly Cys-COOH;
(SEQ ID NO: 50)
H-Ag Cys Asp Leu Cys Ag Asn Pro Ala Cys Tyr Gly Cys-COOH;
(SEQ ID NO: 51)
H-Ag Cys Ser Leu Cys Ag Asn Pro Ala Cys Tyr Gly Cys-COOH;
(SEQ ID NO: 52)
H-Ag Cys Thr Leu Cys Ag Asn Pro Ala Cys Tyr Gly Cys-COOH;
(SEQ ID NO: 53)
H-Hag Cys Glu Leu Cys Hag Asn Pro Ala Cys Thr Gly Cys Tyr-NH₂;
(SEQ ID NO: 54)
H-Ag Cys Gln Leu Cys Ag Asn Pro Ala Cys Tyr Gly Cys-COOH;
(SEQ ID NO: 55)
H-Ag Cys Glu Leu Cys Ag Leu Pro Ala Cys Thr Gly Cys Tyr-NH₂;
(SEQ ID NO: 56)
Pent-Cys Glu Leu Cys Ag Asn Pro Ala Cys Tyr Gly Cys Tyr-NH₂;
(SEQ ID NO: 57)
H-Cys Cys Glu Leu Cys Ag Asn Pro Ala Cys Tyr Gly Ag-COOH;
(SEQ ID NO: 58)
Pent-Cys Glu Leu Cys Ag Asn Pro Ala Cys Tyr Gly Cys-COOH;
(SEQ ID NO: 59)
H-Hag Cys Glu Leu Cys Hag Asn Pro Ala Cys Tyr Gly Cys-COOH;
(SEQ ID NO: 60)
H-Ag Cys Glu Leu Cys Ag Asn Val Ala Cys Tyr Gly Cys Tyr-NH₂;
(SEQ ID NO: 61)
H-Ag Cys Glu Leu Cys Ag Asn Val Ala Cys Tyr Gly Cys-COOH;
(SEQ ID NO: 62)
H-Cys Cth Glu Leu Cys Cys Asn Pro Ala Cys Tyr Gly Cys Tyr-NH₂;
(SEQ ID NO: 63)
H-Cth Cys Glu Leu Cys Cys Asn Pro Ala Cys Tyr Gly Cys Tyr-NH₂;
(SEQ ID NO: 64)
H-Cth Cys Glu Leu Cys Cys Asn Pro Ala Cys Tyr Gly Cys-COOH;
(SEQ ID NO: 65)
H-Cys Cth Glu Leu Cys Cys Asn Val Ala Cys Tyr Gly Cys Tyr-NH₂;
(SEQ ID NO: 66)
H-Cys Cth Glu Leu Cys Cys Asn Val Ala Cys Thr Gly Cys Tyr-NH₂;
(SEQ ID NO: 67)
Ac-Cys Cth Glu Leu Cys Cys Asn Val Ala Cys Thr Gly Cys Tyr-NH₂;
(SEQ ID NO: 68)
Ac-Cys Cth Glu Leu Cys Cys Asn Pro Ala Cys Tyr Gly Cys Tyr-NH₂;
(SEQ ID NO: 69)
Ac-Cys Cth Glu Leu Cys Cys Asn Val Ala Cys Tyr Gly Cys-COOH;
(SEQ ID NO: 70)
H-Cys Cth Glu Leu Cys Cys Asn Val Ala Cys Tyr Gly Cys-COOH;
(SEQ ID NO: 71)
Ac-Cys Cth Glu Leu Cys Cys Asn Pro Ala Cys Tyr Gly Cys-COOH;
(SEQ ID NO: 72)
H-Cys Cys Glu Leu Cys Asn Val Ala Cth Tyr Gly Cys-COOH;
(SEQ ID NO: 73)
H-Cys Cys Glu Leu Cys Cys Asn Val Ala Cth Tyr Gly Cys Tyr-NH₂;
(SEQ ID NO: 74)
H-Cys Cys Glu Leu Cys Cys Asn Val Ala Cth Thr Gly Cys Tyr-NH₂;
(SEQ ID NO: 75)
H-Cys Cys Glu Leu Cys Cth Asn Val Ala Cys Tyr Gly Cys-COOH;
(SEQ ID NO: 76)
H-Cys Cys Glu Leu Cys Cth Asn Val Ala Cys Tyr Gly Cys Tyr-NH₂;
(SEQ ID NO: 77)
H-Cys Cys Glu Leu Cys Cth Asn Val Ala Cys Thr Gly Cys Tyr-NH₂;
(SEQ ID NO: 78)
4-Mepip-Cys Cth Glu Leu Cys Cys Asn Val Ala Cys Tyr Gly Cys-COOH;
(SEQ ID NO: 79)
H-Cys Cth Glu Leu Cys Cys Asn Val Ala Cys Thr Gly Cys-COOH;
(SEQ ID NO: 80)
H-Cys Cth Glu Leu Cys Cys Asn Val Ala Cys Thr Gly Cys-NH₂;
(SEQ ID NO: 81)
H-Cys Cth Glu Leu Cys Cys Asn Val Ala Cys Phe Gly Cys-COOH;
(SEQ ID NO: 82)
H-Ag Ag Glu Leu Ag Ag Asn Pro Ala Ag Thr Gly Ag Tyr-COOH;
(SEQ ID NO: 83)
H-Cys Cth Glu Leu Cys Cys Asn Ala Ala Cys Thr Gly Cys Tyr-NH₂;
(SEQ ID NO: 84)
H-Asp Cys Glu Leu Cys Dpr Asn Val Ala Cys Thr Gly Cys Tyr-NH₂;
(SEQ ID NO: 85)
H-Cys Cth Glu Leu Cys Cys Asn Val Ala Cys Ser Gly Cys Tyr-NH₂;
(SEQ ID NO: 86)
H-Cth Glu Leu Cys Ag Asn Val Ala Cys Thr Gly Cys Tyr-NH₂;
(SEQ ID NO: 87)
H-Ag Cys Glu Leu Cys Ag Asn Val Ala Cys Thr Gly Cys Tyr-NH₂;
(SEQ ID NO: 88)
H-Ag Cys Glu Leu Cys Ag Asn Pro Ala Cys Thr Gly Cys Tyr-NH₂;
(SEQ ID NO: 89)
H-Cth Cys Glu Leu Cys Cys Asn Val Ala Cys Thr Gly Cys-COOH;
(SEQ ID NO: 90)
H-Cth Cys Glu Leu Cys Cys Asn Val Ala Cys Thr Gly Cys Tyr-NH₂;
(SEQ ID NO: 91)
H-Cth Cys Glu Leu Cys Cys Asn Val Ala Cys Tyr Gly Cys Tyr-NH₂;
or
(SEQ ID NO: 92)
H-Cys Cys Glu Leu Cys Cys Asn Val Ala Cth Tyr Gly Cys-COOH.

17. A peptide or a pharmaceutically acceptable salt thereof, wherein the peptide consists of the amino acid sequence
Xaa₁ Xaa₂ Xaa₃ Xaa₄ Xaa₅ Xaa₆ Xaa₇ Xaa₈ Xaa₉ Xaa₁₀ Xaa₁₁ Xaa₁₂ Xaa₁₃ Xaa₁₄ Ala₁₅ Xaa₁₆ Xaa₁₇ Gly₁₈ Xaa₁₉ Xaa₂₀ Xaa₂₁ (SEQ ID NO: 1), or a pharmaceutically acceptable salt thereof; wherein
Xaa₁ is BE or is absent;
Xaa₂ is BK, Asn or is absent;
Xaa₃ is Asn, Ser or is absent;
Xaa₄ is Ser or is absent;
Xaa₅ is Ser, Asn, Ile, BE or is absent;
Xaa₆ is Tyr, Asp, 4-fluorophenylalanine ((4-F)Phe), BK or is absent;
Xaa₇ is Cys, cystathionine (Cth), allylglycine (Ag), Hag, or Asp;
Xaa₈ is Cys, cystathionine (Cth), penicillamine (Pen), or allylglycine (Ag);
Xaa₉ is Glu, Asp, Ser, Thr, or Gln;
Xaa₁₀ is Leu, cyclohexylalanine (Cha), Phe, or 4-fluorophenylalanine ((4-F)Phe);
Xaa₁₁ is Cys, Ag, or penicillamine (Pen);
Xaa₁₂ is Cys, allylglycine (Ag), Hag, Cth, Dpr, or Val;
Xaa₁₃ is Asn or Leu;
Xaa₁₄ is Pro, Val, sarcosine (Sar), Leu, or Hydroxyproline (OH-Pro);
Xaa₁₆ is Cys, Ag, Pen or Cth;
Xaa₁₇ is Tyr, Thr, cyclohexylalanine (Cha), 4-fluorophenylalanine ((4-F)Phe), Phe, Ser, or Ala;
Xaa₁₉ is Cys, Ag or Pen;
Xaa₂₀ is Tyr, Leu, 4-fluorophenylalanine ((4-F)Phe), cyclohexylalanine (Cha), D-Tyr, N-Methyl Tyr (Nme-Tyr) or is absent;
Xaa₂₁ is absent or Asn;
wherein at least one Xaa is BE, BK, (4-F)Phe, Cth, Ag, Hag, Pen, Cha, Sar, Dpr, or OH-Pro; and
wherein the peptide contains a covalent bond between Xaa₇ and Xaa₁₂, Xaa₈ and Xaa₁₆ and Xaa₁₁ and Xaa₁₉.

18. The peptide or pharmaceutically acceptable salt thereof according to claim 17, wherein the N-terminus is capped with acetic acid, pentenoic acid, biotin, 4-Mepip, C12 alkyl carboxylic acid, C14 alkyl carboxylic acid, C16 alkyl carboxylic acid, or C18 alkyl carboxylic acid.

19. The peptide or pharmaceutically acceptable salt thereof according to any one of claims 17 or 18, wherein the C-terminus is amidated.

20. The peptide or pharmaceutically acceptable salt thereof according to any one of claims 17-19, wherein
Xaa₇ and Xaa₁₂ are both Ag and a dicarba bond is present between Ag₇ and Ag₁₂;
Xaa₇ and Xaa₁₂ are both Cys and a disulfide bond is present between Cys₇ and Cys₁₂;
Xaa₈ and Xaa₁₆ are both Cys and a disulfide bond is present between Cys₈ and Cys₁₆;
Xaa₇ is Cth and Xaa₁₂ is Cys and a bond is present between Cth₇ and Cys₁₂;
Xaa₈ is Cth and Xaa₁₆ is Cys and a bond is present between Cth₈ and Cys₁₆;
Xaa₁₁ and Xaa₁₉ are Cys and a disulfide bond is present between Cys₁₁ and Cys₁₉; or
any combination thereof.

21. The peptide or pharmaceutically acceptable salt thereof according to any one of claims 17-19, wherein Xaa₇ is allylglycine or Cys.

22. The peptide or pharmaceutically acceptable salt thereof according to any one of claims 17-19, wherein Xaa₈ is Cys or cystathionine.

23. The peptide or pharmaceutically acceptable salt thereof according to any one of claims 17-22, wherein Xaa₉ is Glu.

24. The peptide or pharmaceutically acceptable salt thereof according to any one of claims 17-23, wherein Xaa₁₀ is Leu.

25. The peptide or pharmaceutically acceptable salt thereof according to any one of claims 17-24, wherein Xaa₁₂ is Cys or allylglycine.

26. The peptide or pharmaceutically acceptable salt thereof according to any one of claims 17-25, wherein Xaa₁₄ is Val or Pro.

27. The peptide or pharmaceutically acceptable salt thereof according to any one of claims 17-26, wherein Xaa₁₇ is Tyr or Thr.

28. The peptide or pharmaceutically acceptable salt thereof according to any one of claims 17-27, wherein Xaa₂₀ is Tyr or is absent.

29. The peptide or pharmaceutically acceptable salt thereof according to any one of claims 17-28, wherein Xaa₂₁ is absent.

30. The peptide or pharmaceutically acceptable salt thereof according to claim 17, wherein:
Xaa₁ is absent;
Xaa₂ is absent;
Xaa₃ is absent;
Xaa₄ is absent;
Xaa₅ is absent;
Xaa₆ is absent;
Xaa₇ is Ag, Cys, or Cth;
Xaa₈ is Cys or Cth;
Xaa₉ is Glu;
Xaa₁₀ is Leu;
Xaa₁₂ is Ag or Cys;
Xaa₁₄ is Val or Pro;
Xaa₁₇ is Tyr or Thr; and
Xaa₂₀ is Tyr or is absent.

31. The peptide or pharmaceutically acceptable salt thereof according to claim 30, wherein the peptide consists of the amino acid sequence
(SEQ ID NO: 2)
H-Ag Cys Glu Leu Cys Ag Asn Pro Ala Cys Thr Gly Cys Tyr-NH₂;
(SEQ ID NO: 26)
H-Ag Cys Glu Leu Cys Ag Asn Pro Ala Cys Tyr Gly Cys Tyr-NH₂;
(SEQ ID NO: 43)
H-Ag Cys Glu Leu Cys Ag Asn Pro Ala Cys Tyr Gly Cys-COOH;
(SEQ ID NO: 47)
H-Ag Cys Glu Leu Cys Ag Asn Val Ala Cys Thr Gly Cys Tyr-NH₂;
(SEQ ID NO: 62)
H-Ag Cys Glu Leu Cys Ag Asn Val Ala Cys Tyr Gly Cys Tyr-NH₂;
(SEQ ID NO: 64)
H-Cth Cys Glu Leu Cys Cys Asn Pro Ala Cys Tyr Gly Cys-COOH;
(SEQ ID NO: 68)
Ac-Cys Cth Glu Leu Cys Cys Asn Pro Ala Cys Tyr Gly Cys Tyr-NH₂;
(SEQ ID NO: 69)
Ac-Cys Cth Glu Leu Cys Cys Asn Val Ala Cys Tyr Gly Cys-COOH;
(SEQ ID NO: 70)
H-Cys Cth Glu Leu Cys Cys Asn Val Ala Cys Tyr Gly Cys-COOH;
or
(SEQ ID NO: 71)
Ac-Cys Cth Glu Leu Cys Cys Asn Pro Ala Cys Tyr Gly Cys-COOH.

32. The peptide or a pharmaceutically acceptable salt thereof according to claim 17, wherein the peptide consists of the amino acid sequence:
(SEQ ID NO: 2)
H-Ag Cys Glu Leu Cys Ag Asn Pro Ala Cys Thr Gly Cys Tyr-NH₂;
(SEQ ID NO: 3)
C12-BE BK Asn Ser Ser Tyr Cys Cys Glu Leu Cys Cys Asn Pro Ala Cys
Thr Gly Cys Tyr-NH₂;
(SEQ ID NO: 4)
C16-BE BK Asn Ser Ser Tyr Cys Cys Glu Leu Cys Cys Asn Pro Ala Cys
Thr Gly Cys Tyr-NH₂;
(SEQ ID NO: 5)
C12-BE BK Asn Ser Ser Tyr Cys Cys Glu Leu Cys Cys Asn Pro Ala Cys
Thr Gly Cys Tyr-COOH;
(SEQ ID NO: 6)
C14-BE BK Asn Ser Ser Tyr Cys Cys Glu Leu Cys Cys Asn Pro Ala Cys
Thr Gly Cys Tyr-COOH;
(SEQ ID NO: 7)
C16-BE BK Asn Ser Ser Tyr Cys Cys Glu Leu Cys Cys Asn Pro Ala Cys
Thr Gly Cys Tyr-COOH;
(SEQ ID NO: 8)
H-Cys Ag Glu Leu Cys Cys Asn Pro Ala Ag Thr Gly Cys Tyr-NH₂;
(SEQ ID NO: 9)
H-Asn Asp Asp Ag Glu Leu Cys Val Asn Val Ala Ag Thr Gly Cys Leu-
NH₂;
(SEQ ID NO: 10)
H-Asn Asp Asp Cys Glu Leu Ag Val Asn Val Ala Cys Thr Gly Ag Leu-
NH₂;
(SEQ ID NO: 11)
C18-BE BK Asn Ser Ser Tyr Cys Cys Glu Leu Cys Cys Asn Pro Ala Cys
Thr Gly Cys Tyr-COOH;
(SEQ ID NO: 12)
H-Ag Cys Glu Leu Cys Ag Asn Pro Ala Cys Thr Gly Cys-NH₂;
(SEQ ID NO: 13)
H-Ag Cys Glu Leu Cys Ag Asn Pro Ala Cys Ala Gly Cys-NH₂;
(SEQ ID NO: 14)
H-Ag Cys Glu Phe Cys Ag Asn Pro Ala Cys Thr Gly Cys Tyr-NH₂;
(SEQ ID NO: 15)
H-Ag Cys Glu (4-F)Phe Cys Ag Asn Pro Ala Cys Thr Gly Cys-NH₂;
(SEQ ID NO: 16)
H-Ag Cys Glu (4-F)Phe Cys Ag Asn Pro Ala Cys Thr Gly Cys Tyr-NH₂;
(SEQ ID NO: 17)
H-Ag Cys Glu (4-F)Phe Cys Ag Asn Pro Ala Cys Thr Gly Cys (4-F)Phe-
NH₂;
(SEQ ID NO: 18)
H-Cys Cys Glu Leu Ag Cys Asn Pro Ala Cys Thr Gly Ag Tyr-NH₂;
(SEQ ID NO: 19)
H-Ag Cys Glu Phe Cys Ag Asn Pro Ala Cys Thr Gly Cys-NH₂;
(SEQ ID NO: 20)
H-Ag Cys Glu Phe Cys Ag Asn Pro Ala Cys Thr Gly Cys (4-F)Phe-NH₂;
(SEQ ID NO: 21)
H-Cys Cys Glu (4-F)Phe Cys Cys Asn Pro Ala Cys Thr Gly Cys Tyr-NH₂;
(SEQ ID NO: 22)
H-Cys Cys Glu (4-F)Phe Cys Cys Asn Pro Ala Cys Thr Gly Cys (4-F)Phe-
NH₂;
(SEQ ID NO: 23)
H-Ag Cys Glu Leu Cys Ag Asn Pro Ala Cys Thr Gly Cys (4-F)Phe-NH₂;
(SEQ ID NO: 24)
H-(4-F)Phe Ag Cys Glu Leu Cys Ag Asn Pro Ala Cys Thr Gly Cys (4-
F)Phe-NH₂;
(SEQ ID NO: 25)
H-Ag Cys Glu Leu Cys Ag Asn Pro Ala Cys Ala Gly Cys Tyr-NH₂;
(SEQ ID NO: 26)
H-Ag Cys Glu Leu Cys Ag Asn Pro Ala Cys Tyr Gly Cys Tyr-NH₂;
(SEQ ID NO: 27)
H-Ag Cys Glu Leu Cys Ag Asn Pro Ala Cys Phe Gly Cys Tyr-NH₂;
(SEQ ID NO: 28)
H-Ag Cys Glu Leu Cys Ag Asn Pro Ala Cys (4-F)Phe Gly Cys Tyr-NH₂;
(SEQ ID NO: 29)
H-Ag Cys Glu Leu Cys Ag Asn Pro Ala Cys Thr Gly Cys Leu-NH₂;
(SEQ ID NO: 30)
H-Ag Cys Glu Leu Cys Ag Asn Pro Ala Cys Thr Gly Cys Cha-NH₂;
(SEQ ID NO: 31)
H-Ag Cys Glu Cha Cys Ag Asn Pro Ala Cys Thr Gly Cys Tyr-NH₂;
(SEQ ID NO: 32)
H-Ag Cys Glu Leu Cys Ag Asn Pro Ala Cys Thr Gly Cys D-Tyr-NH₂;
(SEQ ID NO: 33)
H-Ag Pen Glu Leu Cys Ag Asn Pro Ala Pen Thr Gly Cys Tyr-NH₂;
(SEQ ID NO: 34)
H-Ag Cys Glu Leu Cys Ag Asn Pro Ala Cys Thr Gly Cys Leu Asn-NH₂;
(SEQ ID NO: 35)
H-Ag Cys Glu Leu Cys Ag Asn Pro Ala Cys Thr Gly Cys Tyr Asn-NH₂;
(SEQ ID NO: 36)
H-Asp Ag Cys Glu Leu Cys Ag Asn Pro Ala Cys Thr Gly Cys Tyr-NH₂;
(SEQ ID NO: 37)
H-Ile Asp Ag Cys Glu Leu Cys Ag Asn Pro Ala Cys Thr Gly Cys Tyr-
NH₂;
(SEQ ID NO: 38)
H-Ag Cys Glu Leu Cys Ag Asn OH-Pro Ala Cys Thr Gly Cys Tyr-NH₂;
(SEQ ID NO: 39)
H-Ag Cys Glu Leu Cys Ag Asn OH-Pro Ala Cys Thr Gly Cys-NH₂;
(SEQ ID NO: 40)
H-Ag Cys Glu Leu Pen Ag Asn Pro Ala Cys Thr Gly Pen Tyr-NH₂;
(SEQ ID NO: 41)
H-Ag Cys Glu Leu Pen Ag Asn Pro Ala Cys Thr Gly Pen Tyr-NH₂;
(SEQ ID NO: 42)
H-Ag Cys Glu Leu Cys Ag Asn Pro Ala Cys Tyr Gly Cys Nme-Tyr-NH₂;
(SEQ ID NO: 43)
H-Ag Cys Glu Leu Cys Ag Asn Pro Ala Cys Tyr Gly Cys-COOH;
(SEQ ID NO: 44)
H-Ag Cys Glu Leu Cys Ag Asn Pro Ala Cys Cha Gly Cys Tyr-NH₂;
(SEQ ID NO: 45)
H-Ag Cys Glu Leu Cys Ag Asn Leu Ala Cys Thr Gly Cys Tyr-NH₂;
(SEQ ID NO: 46)
H-Ag Cys Glu Leu Cys Ag Asn Sar Ala Cys Thr Gly Cys Tyr-NH₂;
(SEQ ID NO: 47)
H-Ag Cys Glu Leu Cys Ag Asn Val Ala Cys Thr Gly Cys Tyr-NH₂;
(SEQ ID NO: 48)
H-Ag Cys Glu Leu Cys Ag Asn Pro Ala Cys Tyr Gly Cys Nme-Tyr-NH₂;
(SEQ ID NO: 49)
H-Ag Cys Glu Leu Cys Ag Leu Pro Ala Cys Tyr Gly Cys-COOH;
(SEQ ID NO: 50)
H-Ag Cys Asp Leu Cys Ag Asn Pro Ala Cys Tyr Gly Cys-COOH;
(SEQ ID NO: 51)
H-Ag Cys Ser Leu Cys Ag Asn Pro Ala Cys Tyr Gly Cys-COOH;
(SEQ ID NO: 52)
H-Ag Cys Thr Leu Cys Ag Asn Pro Ala Cys Tyr Gly Cys-COOH;
(SEQ ID NO: 53)
H-Hag Cys Glu Leu Cys Hag Asn Pro Ala Cys Thr Gly Cys Tyr-NH₂;
(SEQ ID NO: 54)
H-Ag Cys Gln Leu Cys Ag Asn Pro Ala Cys Tyr Gly Cys-COOH;
(SEQ ID NO: 55)
H-Ag Cys Glu Leu Cys Ag Leu Pro Ala Cys Thr Gly Cys Tyr-NH₂;
(SEQ ID NO: 56)
Pent-Cys Glu Leu Cys Ag Asn Pro Ala Cys Tyr Gly Cys Tyr-NH₂;
(SEQ ID NO: 57)
H-Cys Cys Glu Leu Cys Ag Asn Pro Ala Cys Tyr Gly Ag-COOH;
(SEQ ID NO: 58)
Pent-Cys Glu Leu Cys Ag Asn Pro Ala Cys Tyr Gly Cys-COOH;
(SEQ ID NO: 59)
H-Hag Cys Glu Leu Cys Hag Asn Pro Ala Cys Tyr Gly Cys-COOH;
(SEQ ID NO: 60)
H-Ag Cys Glu Leu Cys Ag Asn Val Ala Cys Tyr Gly Cys Tyr-NH₂;
(SEQ ID NO: 61)
H-Ag Cys Glu Leu Cys Ag Asn Val Ala Cys Tyr Gly Cys-COOH;
(SEQ ID NO: 62)
H-Cys Cth Glu Leu Cys Cys Asn Pro Ala Cys Tyr Gly Cys Tyr-NH₂;
(SEQ ID NO: 63)
H-Cth Cys Glu Leu Cys Cys Asn Pro Ala Cys Tyr Gly Cys Tyr-NH₂;
(SEQ ID NO: 64)
H-Cth Cys Glu Leu Cys Cys Asn Pro Ala Cys Tyr Gly Cys-COOH;
(SEQ ID NO: 65)
H-Cys Cth Glu Leu Cys Cys Asn Val Ala Cys Tyr Gly Cys Tyr-NH₂;
(SEQ ID NO: 66)
H-Cys Cth Glu Leu Cys Cys Asn Val Ala Cys Thr Gly Cys Tyr-NH₂;
(SEQ ID NO: 67)
Ac-Cys Cth Glu Leu Cys Cys Asn Val Ala Cys Thr Gly Cys Tyr-NH₂;
(SEQ ID NO: 68)
Ac-Cys Cth Glu Leu Cys Cys Asn Pro Ala Cys Tyr Gly Cys Tyr-NH₂;
(SEQ ID NO: 69)
Ac-Cys Cth Glu Leu Cys Cys Asn Val Ala Cys Tyr Gly Cys-COOH;
(SEQ ID NO: 70)
H-Cys Cth Glu Leu Cys Cys Asn Val Ala Cys Tyr Gly Cys-COOH;
(SEQ ID NO: 71)
Ac-Cys Cth Glu Leu Cys Cys Asn Pro Ala Cys Tyr Gly Cys-COOH;
(SEQ ID NO: 72)
H-Cys Cys Glu Leu Cys Asn Val Ala Cth Tyr Gly Cys-COOH;
(SEQ ID NO: 73)
H-Cys Cys Glu Leu Cys Cys Asn Val Ala Cth Tyr Gly Cys Tyr-NH₂;
(SEQ ID NO: 74)
H-Cys Cys Glu Leu Cys Cys Asn Val Ala Cth Thr Gly Cys Tyr-NH₂;
(SEQ ID NO: 75)
H-Cys Cys Glu Leu Cys Cth Asn Val Ala Cys Tyr Gly Cys-COOH;
(SEQ ID NO: 76)
H-Cys Cys Glu Leu Cys Cth Asn Val Ala Cys Tyr Gly Cys Tyr-NH₂;
(SEQ ID NO: 77)
H-Cys Cys Glu Leu Cys Cth Asn Val Ala Cys Thr Gly Cys Tyr-NH₂;
(SEQ ID NO: 78)
4-Mepip-Cys Cth Glu Leu Cys Cys Asn Val Ala Cys Tyr Gly Cys-COOH;
(SEQ ID NO: 79)
H-Cys Cth Glu Leu Cys Cys Asn Val Ala Cys Thr Gly Cys-COOH;
(SEQ ID NO: 80)
H-Cys Cth Glu Leu Cys Cys Asn Val Ala Cys Thr Gly Cys-NH₂;
(SEQ ID NO: 81)
H-Cys Cth Glu Leu Cys Cys Asn Val Ala Cys Phe Gly Cys-COOH;
(SEQ ID NO: 82)
H-Ag Ag Glu Leu Ag Ag Asn Pro Ala Ag Thr Gly Ag Tyr-COOH;
(SEQ ID NO: 83)
H-Cys Cth Glu Leu Cys Cys Asn Ala Ala Cys Thr Gly Cys Tyr-NH₂;
(SEQ ID NO: 84)
H-Asp Cys Glu Leu Cys Dpr Asn Val Ala Cys Thr Gly Cys Tyr-NH₂;
(SEQ ID NO: 85)
H-Cys Cth Glu Leu Cys Cys Asn Val Ala Cys Ser Gly Cys Tyr-NH₂;
(SEQ ID NO: 86)
H-Cth Glu Leu Cys Ag Asn Val Ala Cys Thr Gly Cys Tyr-NH₂;
(SEQ ID NO: 87)
H-Ag Cys Glu Leu Cys Ag Asn Val Ala Cys Thr Gly Cys Tyr-NH₂;
(SEQ ID NO: 88)
H-Ag Cys Glu Leu Cys Ag Asn Pro Ala Cys Thr Gly Cys Tyr-NH₂;
(SEQ ID NO: 89)
H-Cth Cys Glu Leu Cys Cys Asn Val Ala Cys Thr Gly Cys-COOH;
(SEQ ID NO: 90)
H-Cth Cys Glu Leu Cys Cys Asn Val Ala Cys Thr Gly Cys Tyr-NH₂;
(SEQ ID NO: 91)
H-Cth Cys Glu Leu Cys Cys Asn Val Ala Cys Tyr Gly Cys Tyr-NH₂;
or
(SEQ ID NO: 92)
H-Cys Cys Glu Leu Cys Cys Asn Val Ala Cth Tyr Gly Cys-COOH.

33. A peptide or a pharmaceutically acceptable salt thereof, wherein the peptide comprises the amino acid sequence:
Xaa₁ Xaa₂ Xaa₃ Xaa₄ Xaa₅ Xaa₆ Xaa₇ Xaa₈ Xaa₉ Xaa₁₀ Cys₁₁ Xaa₁₂ Asn₁₃ Xaa₁₄ Ala₁₅ Cys₁₆ Xaa₁₇ Gly₁₈ Cys₁₉ Xaa₂₀ Xaa₂₁ (SEQ ID NO: 93), or a pharmaceutically acceptable salt thereof; wherein
Xaa₁ is BE or is absent;
Xaa₂ is BK or is absent;
Xaa₃ is Asn or is absent;
Xaa₄ is Ser or is absent;
Xaa₅ is Ser, Asn, Ile, or is absent;
Xaa₆ is Tyr, Asp, 4-fluorophenylalanine ((4-F)Phe), or is absent;
Xaa₇ is Cys, cystathionine (Cth), allylglycine (Ag), Hag, or Asp;
Xaa₈ is Cys, cystathionine (Cth), penicillamine (Pen), or allylglycine (Ag);
Xaa₉ is Glu, Asp, Ser, Thr, or Gln;
Xaa₁₀ is Leu, cyclohexylalanine (Cha), Phe, or 4-fluorophenylalanine ((4-F)Phe);
Xaa₁₂ is Cys, allylglycine (Ag), Hag, or Val;
Xaa₁₄ is Pro, Val, sarcosine (Sar), Leu, or Hydroxyproline (OH-Pro);
Xaa₁₇ is Tyr, Thr, cyclohexylalanine (Cha), 4-fluorophenylalanine ((4-F)Phe), Phe, or Ala;
Xaa₂₀ is Tyr, Leu, 4-fluorophenylalanine ((4-F)Phe), cyclohexylalanine (Cha), D-Tyr, N-Methyl Tyr (Nme-Tyr) or is absent;
Xaa₂₁ is absent or Asn;
wherein at least one Xaa is BE, BK, (4-F)Phe, Cth, Ag, Hag, Pen, Cha, Sar, or OH-Pro; and
wherein the peptide contains a covalent bond between Xaa₇ and Xaa₁₂, Xaa₈ and Cys₁₆, and Cys₁₁ and Cys₁₉.

34. The peptide or pharmaceutically acceptable salt thereof according to claim 33, wherein the N-terminus is capped with acetic acid, pentenoic acid, 4-Mepip, C12 alkyl carboxylic acid, C14 alkyl carboxylic acid, C16 alkyl carboxylic acid, or C18 alkyl carboxylic acid.

35. The peptide or pharmaceutically acceptable salt thereof according to claim 33, wherein the C-terminus is amidated.

36. A pharmaceutical composition comprising a peptide or a pharmaceutically acceptable salt thereof, and at least one pharmaceutically acceptable excipient, diluent or carrier, wherein the peptide or pharmaceutically acceptable salt thereof comprises the amino acid sequence:
Xaa₁ Xaa₂ Xaa₃ Xaa₄ Xaa₅ Xaa₆ Xaa₇ Xaa₈ Xaa₉ Xaa₁₀ Xaa₁₁ Xaa₁₂ Xaa₁₃ Xaa₁₄ Ala₁₅ Xaa₁₆ Xaa₁₇ Gly₁₈ Xaa₁₉ Xaa₂₀ Xaa₂₁ (SEQ ID NO: 1), or a pharmaceutically acceptable salt thereof; wherein
Xaa₁ is BE or is absent;
Xaa₂ is BK, Asn or is absent;
Xaa₃ is Asn, Ser or is absent;
Xaa₄ is Ser or is absent;
Xaa₅ is Ser, Asn, Ile, BE or is absent;
Xaa₆ is Tyr, Asp, 4-fluorophenylalanine ((4-F)Phe), BK or is absent;
Xaa₇ is Cys, cystathionine (Cth), allylglycine (Ag), 4-pentenoic acid (Pent), Hag, or Asp;
Xaa₈ is Cys, cystathionine (Cth), penicillamine (Pen), or allylglycine (Ag);
Xaa₉ is Glu, Asp, Ser, Thr, or Gln;
Xaa₁₀ is Leu, cyclohexylalanine (Cha), Phe, or 4-fluorophenylalanine ((4-F)Phe);
Xaa₁₁ is Cys, Ag, or penicillamine (Pen);
Xaa₁₂ is Cys, allylglycine (Ag), Hag, Cth, Dpr, or Val;
Xaa₁₃ is Asn or Leu;
Xaa₁₄ is Pro, Val, sarcosine (Sar), Leu, or Hydroxyproline (OH-Pro);
Xaa₁₆ is Cys, Ag, Pen or Cth;
Xaa₁₇ is Tyr, Thr, cyclohexylalanine (Cha), 4-fluorophenylalanine ((4-F)Phe), Phe, Ser, or Ala;
Xaa₁₉ is Cys, Ag or Pen;
Xaa₂₀ is Tyr, Leu, 4-fluorophenylalanine ((4-F)Phe), cyclohexylalanine (Cha), D-Tyr, N-Methyl Tyr (Nme-Tyr) or is absent;
Xaa₂₁ is absent or Asn;
wherein at least one Xaa is BE, BK, (4-F)Phe, Cth, Ag, Hag, Pen, Cha, Sar, Dpr, or OH-Pro; and
wherein the peptide contains a covalent bond between Xaa₇ and Xaa₁₂, Xaa₈ and Xaa₁₆, and Xaa₁₁ and Xaa₁₉.

37. The pharmaceutical composition according to claim 36, wherein the peptide or pharmaceutically acceptable salt thereof consists of the amino acid sequence:
(SEQ ID NO: 2)
H-Ag Cys Glu Leu Cys Ag Asn Pro Ala Cys Thr Gly Cys Tyr-NH₂;
(SEQ ID NO: 3)
C12-BE BK Asn Ser Ser Tyr Cys Cys Glu Leu Cys Cys Asn Pro Ala Cys
Thr Gly Cys Tyr-NH₂;
(SEQ ID NO: 4)
C16-BE BK Asn Ser Ser Tyr Cys Cys Glu Leu Cys Cys Asn Pro Ala Cys
Thr Gly Cys Tyr-NH₂;
(SEQ ID NO: 5)
C12-BE BK Asn Ser Ser Tyr Cys Cys Glu Leu Cys Cys Asn Pro Ala Cys
Thr Gly Cys Tyr-COOH;
(SEQ ID NO: 6)
C14-BE BK Asn Ser Ser Tyr Cys Cys Glu Leu Cys Cys Asn Pro Ala Cys
Thr Gly Cys Tyr-COOH;
(SEQ ID NO: 7)
C16-BE BK Asn Ser Ser Tyr Cys Cys Glu Leu Cys Cys Asn Pro Ala Cys
Thr Gly Cys Tyr-COOH;
(SEQ ID NO: 8)
H-Cys Ag Glu Leu Cys Cys Asn Pro Ala Ag Thr Gly Cys Tyr-NH₂;
(SEQ ID NO: 9)
H-Asn Asp Asp Ag Glu Leu Cys Val Asn Val Ala Ag Thr Gly Cys Leu-
NH₂;
(SEQ ID NO: 10)
H-Asn Asp Asp Cys Glu Leu Ag Val Asn Val Ala Cys Thr Gly Ag Leu-
NH₂;
(SEQ ID NO: 11)
C18-BE BK Asn Ser Ser Tyr Cys Cys Glu Leu Cys Cys Asn Pro Ala Cys
Thr Gly Cys Tyr-COOH;
(SEQ ID NO: 12)
H-Ag Cys Glu Leu Cys Ag Asn Pro Ala Cys Thr Gly Cys-NH₂;
(SEQ ID NO: 13)
H-Ag Cys Glu Leu Cys Ag Asn Pro Ala Cys Ala Gly Cys-NH₂;
(SEQ ID NO: 14)
H-Ag Cys Glu Phe Cys Ag Asn Pro Ala Cys Thr Gly Cys Tyr-NH₂;
(SEQ ID NO: 15)
H-Ag Cys Glu (4-F)Phe Cys Ag Asn Pro Ala Cys Thr Gly Cys-NH₂;
(SEQ ID NO: 16)
H-Ag Cys Glu (4-F)Phe Cys Ag Asn Pro Ala Cys Thr Gly Cys Tyr-NH₂;
(SEQ ID NO: 17)
H-Ag Cys Glu (4-F)Phe Cys Ag Asn Pro Ala Cys Thr Gly Cys (4-F)Phe-
NH₂;
(SEQ ID NO: 18)
H-Cys Cys Glu Leu Ag Cys Asn Pro Ala Cys Thr Gly Ag Tyr-NH₂;
(SEQ ID NO: 19)
H-Ag Cys Glu Phe Cys Ag Asn Pro Ala Cys Thr Gly Cys-NH₂;
(SEQ ID NO: 20)
H-Ag Cys Glu Phe Cys Ag Asn Pro Ala Cys Thr Gly Cys (4-F)Phe-NH₂;
(SEQ ID NO: 21)
H-Cys Cys Glu (4-F)Phe Cys Cys Asn Pro Ala Cys Thr Gly Cys Tyr-NH₂;
(SEQ ID NO: 22)
H-Cys Cys Glu (4-F)Phe Cys Cys Asn Pro Ala Cys Thr Gly Cys (4-F)Phe-
NH₂;
(SEQ ID NO: 23)
H-Ag Cys Glu Leu Cys Ag Asn Pro Ala Cys Thr Gly Cys (4-F)Phe-NH₂;
(SEQ ID NO: 24)
H-(4-F)Phe Ag Cys Glu Leu Cys Ag Asn Pro Ala Cys Thr Gly Cys (4-
F)Phe-NH₂;
(SEQ ID NO: 25)
H-Ag Cys Glu Leu Cys Ag Asn Pro Ala Cys Ala Gly Cys Tyr-NH₂;
(SEQ ID NO: 26)
H-Ag Cys Glu Leu Cys Ag Asn Pro Ala Cys Tyr Gly Cys Tyr-NH₂;
(SEQ ID NO: 27)
H-Ag Cys Glu Leu Cys Ag Asn Pro Ala Cys Phe Gly Cys Tyr-NH₂;
(SEQ ID NO: 28)
H-Ag Cys Glu Leu Cys Ag Asn Pro Ala Cys (4-F)Phe Gly Cys Tyr-NH₂;
(SEQ ID NO: 29)
H-Ag Cys Glu Leu Cys Ag Asn Pro Ala Cys Thr Gly Cys Leu-NH₂;
(SEQ ID NO: 30)
H-Ag Cys Glu Leu Cys Ag Asn Pro Ala Cys Thr Gly Cys Cha-NH₂;
(SEQ ID NO: 31)
H-Ag Cys Glu Cha Cys Ag Asn Pro Ala Cys Thr Gly Cys Tyr-NH₂;
(SEQ ID NO: 32)
H-Ag Cys Glu Leu Cys Ag Asn Pro Ala Cys Thr Gly Cys D-Tyr-NH₂;
(SEQ ID NO: 33)
H-Ag Pen Glu Leu Cys Ag Asn Pro Ala Pen Thr Gly Cys Tyr-NH₂;
(SEQ ID NO: 34)
H-Ag Cys Glu Leu Cys Ag Asn Pro Ala Cys Thr Gly Cys Leu Asn-NH₂;
(SEQ ID NO: 35)
H-Ag Cys Glu Leu Cys Ag Asn Pro Ala Cys Thr Gly Cys Tyr Asn-NH₂;
(SEQ ID NO: 36)
H-Asp Ag Cys Glu Leu Cys Ag Asn Pro Ala Cys Thr Gly Cys Tyr-NH₂;
(SEQ ID NO: 37)
H-Ile Asp Ag Cys Glu Leu Cys Ag Asn Pro Ala Cys Thr Gly Cys Tyr-
NH₂;
(SEQ ID NO: 38)
H-Ag Cys Glu Leu Cys Ag Asn OH-Pro Ala Cys Thr Gly Cys Tyr-NH₂;
(SEQ ID NO: 39)
H-Ag Cys Glu Leu Cys Ag Asn OH-Pro Ala Cys Thr Gly Cys-NH₂;
(SEQ ID NO: 40)
H-Ag Cys Glu Leu Pen Ag Asn Pro Ala Cys Thr Gly Pen Tyr-NH₂;
(SEQ ID NO: 41)
H-Ag Cys Glu Leu Pen Ag Asn Pro Ala Cys Thr Gly Pen Tyr-NH₂;
(SEQ ID NO: 42)
H-Ag Cys Glu Leu Cys Ag Asn Pro Ala Cys Tyr Gly Cys Nme-Tyr-NH₂;
(SEQ ID NO: 43)
H-Ag Cys Glu Leu Cys Ag Asn Pro Ala Cys Tyr Gly Cys-COOH;
(SEQ ID NO: 44)
H-Ag Cys Glu Leu Cys Ag Asn Pro Ala Cys Cha Gly Cys Tyr-NH₂;
(SEQ ID NO: 45)
H-Ag Cys Glu Leu Cys Ag Asn Leu Ala Cys Thr Gly Cys Tyr-NH₂;
(SEQ ID NO: 46)
H-Ag Cys Glu Leu Cys Ag Asn Sar Ala Cys Thr Gly Cys Tyr-NH₂;
(SEQ ID NO: 47)
H-Ag Cys Glu Leu Cys Ag Asn Val Ala Cys Thr Gly Cys Tyr-NH₂;
(SEQ ID NO: 48)
H-Ag Cys Glu Leu Cys Ag Asn Pro Ala Cys Tyr Gly Cys Nme-Tyr-NH₂;
(SEQ ID NO: 49)
H-Ag Cys Glu Leu Cys Ag Leu Pro Ala Cys Tyr Gly Cys-COOH;
(SEQ ID NO: 50)
H-Ag Cys Asp Leu Cys Ag Asn Pro Ala Cys Tyr Gly Cys-COOH;
(SEQ ID NO: 51)
H-Ag Cys Ser Leu Cys Ag Asn Pro Ala Cys Tyr Gly Cys-COOH;
(SEQ ID NO: 52)
H-Ag Cys Thr Leu Cys Ag Asn Pro Ala Cys Tyr Gly Cys-COOH;
(SEQ ID NO: 53)
H-Hag Cys Glu Leu Cys Hag Asn Pro Ala Cys Thr Gly Cys Tyr-NH₂;
(SEQ ID NO: 54)
H-Ag Cys Gln Leu Cys Ag Asn Pro Ala Cys Tyr Gly Cys-COOH;
(SEQ ID NO: 55)
H-Ag Cys Glu Leu Cys Ag Leu Pro Ala Cys Thr Gly Cys Tyr-NH₂;
(SEQ ID NO: 56)
Pent-Cys Glu Leu Cys Ag Asn Pro Ala Cys Tyr Gly Cys Tyr-NH₂;
(SEQ ID NO: 57)
H-Cys Cys Glu Leu Cys Ag Asn Pro Ala Cys Tyr Gly Ag-COOH;
(SEQ ID NO: 58)
Pent-Cys Glu Leu Cys Ag Asn Pro Ala Cys Tyr Gly Cys-COOH;
(SEQ ID NO: 59)
H-Hag Cys Glu Leu Cys Hag Asn Pro Ala Cys Tyr Gly Cys-COOH;
(SEQ ID NO: 60)
H-Ag Cys Glu Leu Cys Ag Asn Val Ala Cys Tyr Gly Cys Tyr-NH₂;
(SEQ ID NO: 61)
H-Ag Cys Glu Leu Cys Ag Asn Val Ala Cys Tyr Gly Cys-COOH;
(SEQ ID NO: 62)
H-Cys Cth Glu Leu Cys Cys Asn Pro Ala Cys Tyr Gly Cys Tyr-NH₂;
(SEQ ID NO: 63)
H-Cth Cys Glu Leu Cys Cys Asn Pro Ala Cys Tyr Gly Cys Tyr-NH₂;
(SEQ ID NO: 64)
H-Cth Cys Glu Leu Cys Cys Asn Pro Ala Cys Tyr Gly Cys-COOH;
(SEQ ID NO: 65)
H-Cys Cth Glu Leu Cys Cys Asn Val Ala Cys Tyr Gly Cys Tyr-NH₂;
(SEQ ID NO: 66)
H-Cys Cth Glu Leu Cys Cys Asn Val Ala Cys Thr Gly Cys Tyr-NH₂;
(SEQ ID NO: 67)
Ac-Cys Cth Glu Leu Cys Cys Asn Val Ala Cys Thr Gly Cys Tyr-NH₂;
(SEQ ID NO: 68)
Ac-Cys Cth Glu Leu Cys Cys Asn Pro Ala Cys Tyr Gly Cys Tyr-NH₂;
(SEQ ID NO: 69)
Ac-Cys Cth Glu Leu Cys Cys Asn Val Ala Cys Tyr Gly Cys-COOH;
(SEQ ID NO: 70)
H-Cys Cth Glu Leu Cys Cys Asn Val Ala Cys Tyr Gly Cys-COOH;
(SEQ ID NO: 71)
Ac-Cys Cth Glu Leu Cys Cys Asn Pro Ala Cys Tyr Gly Cys-COOH;
(SEQ ID NO: 72)
H-Cys Cys Glu Leu Cys Asn Val Ala Cth Tyr Gly Cys-COOH;
(SEQ ID NO: 73)
H-Cys Cys Glu Leu Cys Cys Asn Val Ala Cth Tyr Gly Cys Tyr-NH₂;
(SEQ ID NO: 74)
H-Cys Cys Glu Leu Cys Cys Asn Val Ala Cth Thr Gly Cys Tyr-NH₂;
(SEQ ID NO: 75)
H-Cys Cys Glu Leu Cys Cth Asn Val Ala Cys Tyr Gly Cys-COOH;
(SEQ ID NO: 76)
H-Cys Cys Glu Leu Cys Cth Asn Val Ala Cys Tyr Gly Cys Tyr-NH₂;
(SEQ ID NO: 77)
H-Cys Cys Glu Leu Cys Cth Asn Val Ala Cys Thr Gly Cys Tyr-NH₂;
(SEQ ID NO: 78)
4-Mepip-Cys Cth Glu Leu Cys Cys Asn Val Ala Cys Tyr Gly Cys-COOH;
(SEQ ID NO: 79)
H-Cys Cth Glu Leu Cys Cys Asn Val Ala Cys Thr Gly Cys-COOH;
(SEQ ID NO: 80)
H-Cys Cth Glu Leu Cys Cys Asn Val Ala Cys Thr Gly Cys-NH₂;
(SEQ ID NO: 81)
H-Cys Cth Glu Leu Cys Cys Asn Val Ala Cys Phe Gly Cys-COOH;
(SEQ ID NO: 82)
H-Ag Ag Glu Leu Ag Ag Asn Pro Ala Ag Thr Gly Ag Tyr-COOH;
(SEQ ID NO: 83)
H-Cys Cth Glu Leu Cys Cys Asn Ala Ala Cys Thr Gly Cys Tyr-NH₂;
(SEQ ID NO: 84)
H-Asp Cys Glu Leu Cys Dpr Asn Val Ala Cys Thr Gly Cys Tyr-NH₂;
(SEQ ID NO: 85)
H-Cys Cth Glu Leu Cys Cys Asn Val Ala Cys Ser Gly Cys Tyr-NH₂;
(SEQ ID NO: 86)
H-Cth Glu Leu Cys Ag Asn Val Ala Cys Thr Gly Cys Tyr-NH₂;
(SEQ ID NO: 87)
H-Ag Cys Glu Leu Cys Ag Asn Val Ala Cys Thr Gly Cys Tyr-NH₂;
(SEQ ID NO: 88)
H-Ag Cys Glu Leu Cys Ag Asn Pro Ala Cys Thr Gly Cys Tyr-NH₂;
(SEQ ID NO: 89)
H-Cth Cys Glu Leu Cys Cys Asn Val Ala Cys Thr Gly Cys-COOH;
(SEQ ID NO: 90)
H-Cth Cys Glu Leu Cys Cys Asn Val Ala Cys Thr Gly Cys Tyr-NH₂;
(SEQ ID NO: 91)
H-Cth Cys Glu Leu Cys Cys Asn Val Ala Cys Tyr Gly Cys Tyr-NH₂;
or
(SEQ ID NO: 92)
H-Cys Cys Glu Leu Cys Cys Asn Val Ala Cth Tyr Gly Cys-COOH.

38. The pharmaceutical composition according to claim 36, wherein the peptide or pharmaceutically acceptable salt thereof consists of the amino acid sequence:
(SEQ ID NO: 2)
H-Ag Cys Glu Leu Cys Ag Asn Pro Ala Cys Thr Gly Cys Tyr-NH₂;
(SEQ ID NO: 26)
H-Ag Cys Glu Leu Cys Ag Asn Pro Ala Cys Tyr Gly Cys Tyr-NH₂;
(SEQ ID NO: 43)
H-Ag Cys Glu Leu Cys Ag Asn Pro Ala Cys Tyr Gly Cys-COOH;
(SEQ ID NO: 47)
H-Ag Cys Glu Leu Cys Ag Asn Val Ala Cys Thr Gly Cys Tyr-NH₂;
(SEQ ID NO: 62)
H-Ag Cys Glu Leu Cys Ag Asn Val Ala Cys Tyr Gly Cys Tyr-NH₂;
(SEQ ID NO: 64)
H-Cth Cys Glu Leu Cys Cys Asn Pro Ala Cys Tyr Gly Cys-COOH;
(SEQ ID NO: 68)
Ac-Cys Cth Glu Leu Cys Cys Asn Pro Ala Cys Tyr Gly Cys Tyr-NH₂;
(SEQ ID NO: 69)
Ac-Cys Cth Glu Leu Cys Cys Asn Val Ala Cys Tyr Gly Cys-COOH;
(SEQ ID NO: 70)
H-Cys Cth Glu Leu Cys Cys Asn Val Ala Cys Tyr Gly Cys-COOH;
or
(SEQ ID NO: 71)
Ac-Cys Cth Glu Leu Cys Cys Asn Pro Ala Cys Tyr Gly Cys-COOH.

39. The pharmaceutical composition according to any one of claims 36-38, wherein the N-terminus of the peptide is capped with acetic acid, pentenoic acid, 4-Mepip, C12 alkyl carboxylic acid, C14 alkyl carboxylic acid, C16 alkyl carboxylic acid or C18 alkyl carboxylic acid; the C-terminus of the peptide is amidated; or any combination thereof.

40. The pharmaceutical composition according to any one of claims 36-39, wherein the pharmaceutical composition is a solid dosage formulation.

41. The pharmaceutical composition according to claim 40, wherein the pharmaceutical composition is formulated as an oral solid dosage formulation.

42. A method of cleansing the colon of a subject in preparation for a colonoscopy procedure comprising administering to the subject an effective dose of a colon cleansing composition;
wherein the colon cleansing composition comprises a pharmaceutically acceptable excipient, diluent or carrier, and a peptide or a pharmaceutically acceptable salt thereof, wherein the peptide or a pharmaceutically acceptable salt thereof comprises the amino acid sequence:
Xaa₁ Xaa₂ Xaa₃ Xaa₄ Xaa₅ Xaa₆ Xaa₇ Xaa₈ Xaa₉ Xaa₁₀ Xaa₁₁ Xaa₁₂ Xaa₁₃ Xaa₁₄ Ala₁₅ Xaa₁₆ Xaa₁₇ Gly₁₈ Xaa₁₉ Xaa₂₀ Xaa₂₁ (SEQ ID NO: 1), or a pharmaceutically acceptable salt thereof; wherein
Xaa₁ is BE or is absent;
Xaa₂ is BK, Asn or is absent;
Xaa₃ is Asn, Ser or is absent;
Xaa₄ is Ser or is absent;
Xaa₅ is Ser, Asn, Ile, BE or is absent;
Xaa₆ is Tyr, Asp, 4-fluorophenylalanine ((4-F)Phe), BK or is absent;
Xaa₇ is Cys, cystathionine (Cth), allylglycine (Ag), 4-pentenoic acid (Pent), Hag, or Asp;
Xaa₈ is Cys, cystathionine (Cth), penicillamine (Pen), or allylglycine (Ag);
Xaa₉ is Glu, Asp, Ser, Thr, or Gln;
Xaa₁₀ is Leu, cyclohexylalanine (Cha), Phe, or 4-fluorophenylalanine ((4-F)Phe);
Xaa₁₁ is Cys, Ag, or penicillamine (Pen);
Xaa₁₂ is Cys, allylglycine (Ag), Hag, Cth, Dpr, or Val;
Xaa₁₃ is Asn or Leu;
Xaa₁₄ is Pro, Val, sarcosine (Sar), Leu, or Hydroxyproline (OH-Pro);
Xaa₁₆ is Cys, Ag, Pen or Cth;
Xaa₁₇ is Tyr, Thr, cyclohexylalanine (Cha), 4-fluorophenylalanine ((4-F)Phe), Phe, Ser, or Ala;
Xaa₁₉ is Cys, Ag or Pen;
Xaa₂₀ is Tyr, Leu, 4-fluorophenylalanine ((4-F)Phe), cyclohexylalanine (Cha), D-Tyr, N-Methyl Tyr (Nme-Tyr) or is absent;
Xaa₂₁ is absent or Asn;
wherein at least one Xaa is BE, BK, (4-F)Phe, Cth, Ag, Hag, Pen, Cha, Sar, Dpr, or OH-Pro; and
wherein the peptide contains a covalent bond between Xaa₇ and Xaa₁₂, Xaa₈ and Xaa₁₆, and Xaa₁₁ and Xaa₁₉.

43. The method according to claim 42, wherein the method further comprises:
a. orally or rectally administering to the subject an effective first dose of the colon cleansing composition; and
b. administering to the subject an effective second dose of the colon cleansing composition to substantially cleanse the colon of the subject.

44. The method according to claim 43, wherein the effective second dose is administered the morning after the effective first dose is administered.

45. The method according to claim 42, wherein the colon cleansing composition comprises a peptide or a pharmaceutically acceptable salt thereof comprises the amino acid sequence:
(SEQ ID NO: 2)
H-Ag Cys Glu Leu Cys Ag Asn Pro Ala Cys Thr Gly Cys Tyr-NH₂;
(SEQ ID NO: 3)
C12-BE BK Asn Ser Ser Tyr Cys Cys Glu Leu Cys Cys Asn Pro Ala Cys
Thr Gly Cys Tyr-NH₂;
(SEQ ID NO: 4)
C16-BE BK Asn Ser Ser Tyr Cys Cys Glu Leu Cys Cys Asn Pro Ala Cys
Thr Gly Cys Tyr-NH₂;
(SEQ ID NO: 5)
C12-BE BK Asn Ser Ser Tyr Cys Cys Glu Leu Cys Cys Asn Pro Ala Cys
Thr Gly Cys Tyr-COOH;
(SEQ ID NO: 6)
C14-BE BK Asn Ser Ser Tyr Cys Cys Glu Leu Cys Cys Asn Pro Ala Cys
Thr Gly Cys Tyr-COOH;
(SEQ ID NO: 7)
C16-BE BK Asn Ser Ser Tyr Cys Cys Glu Leu Cys Cys Asn Pro Ala Cys
Thr Gly Cys Tyr-COOH;
(SEQ ID NO: 8)
H-Cys Ag Glu Leu Cys Cys Asn Pro Ala Ag Thr Gly Cys Tyr-NH₂;
(SEQ ID NO: 9)
H-Asn Asp Asp Ag Glu Leu Cys Val Asn Val Ala Ag Thr Gly Cys Leu-
NH₂;
(SEQ ID NO: 10)
H-Asn Asp Asp Cys Glu Leu Ag Val Asn Val Ala Cys Thr Gly Ag Leu-
NH₂;
(SEQ ID NO: 11)
C18-BE BK Asn Ser Ser Tyr Cys Cys Glu Leu Cys Cys Asn Pro Ala Cys
Thr Gly Cys Tyr-COOH;
(SEQ ID NO: 12)
H-Ag Cys Glu Leu Cys Ag Asn Pro Ala Cys Thr Gly Cys-NH₂;
(SEQ ID NO: 13)
H-Ag Cys Glu Leu Cys Ag Asn Pro Ala Cys Ala Gly Cys-NH₂;
(SEQ ID NO: 14)
H-Ag Cys Glu Phe Cys Ag Asn Pro Ala Cys Thr Gly Cys Tyr-NH₂;
(SEQ ID NO: 15)
H-Ag Cys Glu (4-F)Phe Cys Ag Asn Pro Ala Cys Thr Gly Cys-NH₂;
(SEQ ID NO: 16)
H-Ag Cys Glu (4-F)Phe Cys Ag Asn Pro Ala Cys Thr Gly Cys Tyr-NH₂;
(SEQ ID NO: 17)
H-Ag Cys Glu (4-F)Phe Cys Ag Asn Pro Ala Cys Thr Gly Cys (4-F)Phe-
NH₂;
(SEQ ID NO: 18)
H-Cys Cys Glu Leu Ag Cys Asn Pro Ala Cys Thr Gly Ag Tyr-NH₂;
(SEQ ID NO: 19)
H-Ag Cys Glu Phe Cys Ag Asn Pro Ala Cys Thr Gly Cys-NH₂;
(SEQ ID NO: 20)
H-Ag Cys Glu Phe Cys Ag Asn Pro Ala Cys Thr Gly Cys (4-F)Phe-NH₂;
(SEQ ID NO: 21)
H-Cys Cys Glu (4-F)Phe Cys Cys Asn Pro Ala Cys Thr Gly Cys Tyr-NH₂;
(SEQ ID NO: 22)
H-Cys Cys Glu (4-F)Phe Cys Cys Asn Pro Ala Cys Thr Gly Cys (4-F)Phe-
NH₂;
(SEQ ID NO: 23)
H-Ag Cys Glu Leu Cys Ag Asn Pro Ala Cys Thr Gly Cys (4-F)Phe-NH₂;
(SEQ ID NO: 24)
H-(4-F)Phe Ag Cys Glu Leu Cys Ag Asn Pro Ala Cys Thr Gly Cys (4-
F)Phe-NH₂;
(SEQ ID NO: 25)
H-Ag Cys Glu Leu Cys Ag Asn Pro Ala Cys Ala Gly Cys Tyr-NH₂;
(SEQ ID NO: 26)
H-Ag Cys Glu Leu Cys Ag Asn Pro Ala Cys Tyr Gly Cys Tyr-NH₂;
(SEQ ID NO: 27)
H-Ag Cys Glu Leu Cys Ag Asn Pro Ala Cys Phe Gly Cys Tyr-NH₂;
(SEQ ID NO: 28)
H-Ag Cys Glu Leu Cys Ag Asn Pro Ala Cys (4-F)Phe Gly Cys Tyr-NH₂;
(SEQ ID NO: 29)
H-Ag Cys Glu Leu Cys Ag Asn Pro Ala Cys Thr Gly Cys Leu-NH₂;
(SEQ ID NO: 30)
H-Ag Cys Glu Leu Cys Ag Asn Pro Ala Cys Thr Gly Cys Cha-NH₂;
(SEQ ID NO: 31)
H-Ag Cys Glu Cha Cys Ag Asn Pro Ala Cys Thr Gly Cys Tyr-NH₂;
(SEQ ID NO: 32)
H-Ag Cys Glu Leu Cys Ag Asn Pro Ala Cys Thr Gly Cys D-Tyr-NH₂;
(SEQ ID NO: 33)
H-Ag Pen Glu Leu Cys Ag Asn Pro Ala Pen Thr Gly Cys Tyr-NH₂;
(SEQ ID NO: 34)
H-Ag Cys Glu Leu Cys Ag Asn Pro Ala Cys Thr Gly Cys Leu Asn-NH₂;
(SEQ ID NO: 35)
H-Ag Cys Glu Leu Cys Ag Asn Pro Ala Cys Thr Gly Cys Tyr Asn-NH₂;
(SEQ ID NO: 36)
H-Asp Ag Cys Glu Leu Cys Ag Asn Pro Ala Cys Thr Gly Cys Tyr-NH₂;
(SEQ ID NO: 37)
H-Ile Asp Ag Cys Glu Leu Cys Ag Asn Pro Ala Cys Thr Gly Cys Tyr-
NH₂;
(SEQ ID NO: 38)
H-Ag Cys Glu Leu Cys Ag Asn OH-Pro Ala Cys Thr Gly Cys Tyr-NH₂;
(SEQ ID NO: 39)
H-Ag Cys Glu Leu Cys Ag Asn OH-Pro Ala Cys Thr Gly Cys-NH₂;
(SEQ ID NO: 40)
H-Ag Cys Glu Leu Pen Ag Asn Pro Ala Cys Thr Gly Pen Tyr-NH₂;
(SEQ ID NO: 41)
H-Ag Cys Glu Leu Pen Ag Asn Pro Ala Cys Thr Gly Pen Tyr-NH₂;
(SEQ ID NO: 42)
H-Ag Cys Glu Leu Cys Ag Asn Pro Ala Cys Tyr Gly Cys Nme-Tyr-NH₂;
(SEQ ID NO: 43)
H-Ag Cys Glu Leu Cys Ag Asn Pro Ala Cys Tyr Gly Cys-COOH;
(SEQ ID NO: 44)
H-Ag Cys Glu Leu Cys Ag Asn Pro Ala Cys Cha Gly Cys Tyr-NH₂;
(SEQ ID NO: 45)
H-Ag Cys Glu Leu Cys Ag Asn Leu Ala Cys Thr Gly Cys Tyr-NH₂;
(SEQ ID NO: 46)
H-Ag Cys Glu Leu Cys Ag Asn Sar Ala Cys Thr Gly Cys Tyr-NH₂;
(SEQ ID NO: 47)
H-Ag Cys Glu Leu Cys Ag Asn Val Ala Cys Thr Gly Cys Tyr-NH₂;
(SEQ ID NO: 48)
H-Ag Cys Glu Leu Cys Ag Asn Pro Ala Cys Tyr Gly Cys Nme-Tyr-NH₂;
(SEQ ID NO: 49)
H-Ag Cys Glu Leu Cys Ag Leu Pro Ala Cys Tyr Gly Cys-COOH;
(SEQ ID NO: 50)
H-Ag Cys Asp Leu Cys Ag Asn Pro Ala Cys Tyr Gly Cys-COOH;
(SEQ ID NO: 51)
H-Ag Cys Ser Leu Cys Ag Asn Pro Ala Cys Tyr Gly Cys-COOH;
(SEQ ID NO: 52)
H-Ag Cys Thr Leu Cys Ag Asn Pro Ala Cys Tyr Gly Cys-COOH;
(SEQ ID NO: 53)
H-Hag Cys Glu Leu Cys Hag Asn Pro Ala Cys Thr Gly Cys Tyr-NH₂;
(SEQ ID NO: 54)
H-Ag Cys Gln Leu Cys Ag Asn Pro Ala Cys Tyr Gly Cys-COOH;
(SEQ ID NO: 55)
H-Ag Cys Glu Leu Cys Ag Leu Pro Ala Cys Thr Gly Cys Tyr-NH₂;
(SEQ ID NO: 56)
Pent-Cys Glu Leu Cys Ag Asn Pro Ala Cys Tyr Gly Cys Tyr-NH₂;
(SEQ ID NO: 57)
H-Cys Cys Glu Leu Cys Ag Asn Pro Ala Cys Tyr Gly Ag-COOH;
(SEQ ID NO: 58)
Pent-Cys Glu Leu Cys Ag Asn Pro Ala Cys Tyr Gly Cys-COOH;
(SEQ ID NO: 59)
H-Hag Cys Glu Leu Cys Hag Asn Pro Ala Cys Tyr Gly Cys-COOH;
(SEQ ID NO: 60)
H-Ag Cys Glu Leu Cys Ag Asn Val Ala Cys Tyr Gly Cys Tyr-NH₂;
(SEQ ID NO: 61)
H-Ag Cys Glu Leu Cys Ag Asn Val Ala Cys Tyr Gly Cys-COOH;
(SEQ ID NO: 62)
H-Cys Cth Glu Leu Cys Cys Asn Pro Ala Cys Tyr Gly Cys Tyr-NH₂;
(SEQ ID NO: 63)
H-Cth Cys Glu Leu Cys Cys Asn Pro Ala Cys Tyr Gly Cys Tyr-NH₂;
(SEQ ID NO: 64)
H-Cth Cys Glu Leu Cys Cys Asn Pro Ala Cys Tyr Gly Cys-COOH;
(SEQ ID NO: 65)
H-Cys Cth Glu Leu Cys Cys Asn Val Ala Cys Tyr Gly Cys Tyr-NH₂;
(SEQ ID NO: 66)
H-Cys Cth Glu Leu Cys Cys Asn Val Ala Cys Thr Gly Cys Tyr-NH₂;
(SEQ ID NO: 67)
Ac-Cys Cth Glu Leu Cys Cys Asn Val Ala Cys Thr Gly Cys Tyr-NH₂;
(SEQ ID NO: 68)
Ac-Cys Cth Glu Leu Cys Cys Asn Pro Ala Cys Tyr Gly Cys Tyr-NH₂;
(SEQ ID NO: 69)
Ac-Cys Cth Glu Leu Cys Cys Asn Val Ala Cys Tyr Gly Cys-COOH;
(SEQ ID NO: 70)
H-Cys Cth Glu Leu Cys Cys Asn Val Ala Cys Tyr Gly Cys-COOH;
(SEQ ID NO: 71)
Ac-Cys Cth Glu Leu Cys Cys Asn Pro Ala Cys Tyr Gly Cys-COOH;
(SEQ ID NO: 72)
H-Cys Cys Glu Leu Cys Asn Val Ala Cth Tyr Gly Cys-COOH;
(SEQ ID NO: 73)
H-Cys Cys Glu Leu Cys Cys Asn Val Ala Cth Tyr Gly Cys Tyr-NH₂;
(SEQ ID NO: 74)
H-Cys Cys Glu Leu Cys Cys Asn Val Ala Cth Thr Gly Cys Tyr-NH₂;
(SEQ ID NO: 75)
H-Cys Cys Glu Leu Cys Cth Asn Val Ala Cys Tyr Gly Cys-COOH;
(SEQ ID NO: 76)
H-Cys Cys Glu Leu Cys Cth Asn Val Ala Cys Tyr Gly Cys Tyr-NH₂;
(SEQ ID NO: 77)
H-Cys Cys Glu Leu Cys Cth Asn Val Ala Cys Thr Gly Cys Tyr-NH₂;
(SEQ ID NO: 78)
4-Mepip-Cys Cth Glu Leu Cys Cys Asn Val Ala Cys Tyr Gly Cys-COOH;
(SEQ ID NO: 79)
H-Cys Cth Glu Leu Cys Cys Asn Val Ala Cys Thr Gly Cys-COOH;
(SEQ ID NO: 80)
H-Cys Cth Glu Leu Cys Cys Asn Val Ala Cys Thr Gly Cys-NH₂;
(SEQ ID NO: 81)
H-Cys Cth Glu Leu Cys Cys Asn Val Ala Cys Phe Gly Cys-COOH;
(SEQ ID NO: 82)
H-Ag Ag Glu Leu Ag Ag Asn Pro Ala Ag Thr Gly Ag Tyr-COOH;
(SEQ ID NO: 83)
H-Cys Cth Glu Leu Cys Cys Asn Ala Ala Cys Thr Gly Cys Tyr-NH₂;
(SEQ ID NO: 84)
H-Asp Cys Glu Leu Cys Dpr Asn Val Ala Cys Thr Gly Cys Tyr-NH₂;
(SEQ ID NO: 85)
H-Cys Cth Glu Leu Cys Cys Asn Val Ala Cys Ser Gly Cys Tyr-NH₂;
(SEQ ID NO: 86)
H-Cth Glu Leu Cys Ag Asn Val Ala Cys Thr Gly Cys Tyr-NH₂;
(SEQ ID NO: 87)
H-Ag Cys Glu Leu Cys Ag Asn Val Ala Cys Thr Gly Cys Tyr-NH₂;
(SEQ ID NO: 88)
H-Ag Cys Glu Leu Cys Ag Asn Pro Ala Cys Thr Gly Cys Tyr-NH₂;
(SEQ ID NO: 89)
H-Cth Cys Glu Leu Cys Cys Asn Val Ala Cys Thr Gly Cys-COOH;
(SEQ ID NO: 90)
H-Cth Cys Glu Leu Cys Cys Asn Val Ala Cys Thr Gly Cys Tyr-NH₂;
(SEQ ID NO: 91)
H-Cth Cys Glu Leu Cys Cys Asn Val Ala Cys Tyr Gly Cys Tyr-NH₂;
or
(SEQ ID NO: 92)
H-Cys Cys Glu Leu Cys Cys Asn Val Ala Cth Tyr Gly Cys-COOH.

46. The method according to claim 42, wherein the colon cleansing composition comprises a peptide or a pharmaceutically acceptable salt thereof comprises the amino acid sequence:
(SEQ ID NO: 2)
H-Ag Cys Glu Leu Cys Ag Asn Pro Ala Cys Thr Gly Cys Tyr-NH₂;
(SEQ ID NO: 26)
H-Ag Cys Glu Leu Cys Ag Asn Pro Ala Cys Tyr Gly Cys Tyr-NH₂;
(SEQ ID NO: 43)
H-Ag Cys Glu Leu Cys Ag Asn Pro Ala Cys Tyr Gly Cys-COOH;
(SEQ ID NO: 47)
H-Ag Cys Glu Leu Cys Ag Asn Val Ala Cys Thr Gly Cys Tyr-NH₂;
(SEQ ID NO: 62)
H-Ag Cys Glu Leu Cys Ag Asn Val Ala Cys Tyr Gly Cys Tyr-NH₂;
(SEQ ID NO: 64)
H-Cth Cys Glu Leu Cys Cys Asn Pro Ala Cys Tyr Gly Cys-COOH;
(SEQ ID NO: 68)
Ac-Cys Cth Glu Leu Cys Cys Asn Pro Ala Cys Tyr Gly Cys Tyr-NH₂;
(SEQ ID NO: 69)
Ac-Cys Cth Glu Leu Cys Cys Asn Val Ala Cys Tyr Gly Cys-COOH;
(SEQ ID NO: 70)
H-Cys Cth Glu Leu Cys Cys Asn Val Ala Cys Tyr Gly Cys-COOH;
or
(SEQ ID NO: 71)
Ac-Cys Cth Glu Leu Cys Cys Asn Pro Ala Cys Tyr Gly Cys-COOH.

47. A method of producing the peptide of any one of claims 1-35, comprising providing a cell harboring a nucleic acid molecule encoding the polypeptide, culturing the cell under conditions in which the peptide is expressed, and isolating the expressed peptide.

48. A method of producing the peptide of any of claims 1-35, comprising chemically synthesizing the peptide and purifying the synthesized peptide.

49. A method of treating a gastrointestinal or visceral disorder comprising administering to a subject a peptide of any of claims 1-35.

50. A method of treating a disorder selected from colon cancer, Hereditary Nonpolyposis Colorectal Cancer (HNPCC), Lynch syndrome, gastroparesis (GP), polyps, pain, general abdominal pain, post-operative ileus, opioid-induced constipation, functional dyspepsia, diverticular disease including including but not limited to SUDD (symptomatic uncomplicated diverticular disease) and SCAD (segmental colitis associated with diverticulosis), diverticulosis, diarrhea-predominant irritable bowel syndrome, pain associated with irritable bowel syndrom (IBS), ulcerative colitis, ulcerative proctitis, Crohn's Disease, inflammatory bowel disease (IBD), chronic or acute radiation protopathy, rectal pain, chronic proctalgia, proctalgia fugax, anal pain, chronic anal fissure, post-operative anal pain, overactive bladder syndrome, stress incontinence, interstitial cystitis, bladder pain syndrome, colorectal cancer, pain associated with cancer, general pelvic pain, endometriosis, orchialgia, chronic prostatitis, prostatodynia, vulvodynia, urethral syndrome, penile pain, perianal pain and other gastrointestinal and visceral disorders, comprising administering to a subject a peptide of any of claims 1-35.

51. The method of claim 51, further comprising administering to the subject a pharmaceutical composition comprising an active ingredient selected from opioids, tramadol, beta-3 adrenergic receptor agonists, anti-cholinergic agents, and tricyclic antidepressants.

52. A pharmaceutical composition for use in cleansing the colon in a subject in preparation for a colonoscopy procedure, wherein the pharmaceutical composition comprises a pharmaceutically acceptable excipient, diluent or carrier, and a peptide or a pharmaceutically acceptable salt thereof, wherein the peptide or a pharmaceutically acceptable salt thereof comprises the amino acid sequence:
Xaa₁ Xaa₂ Xaa₃ Xaa₄ Xaa₅ Xaa₆ Xaa₇ Xaa₈ Xaa₉ Xaa₁₀ Xaa₁₁ Xaa₁₂ Xaa₁₃ Xaa₁₄ Ala₁₅ Xaa₁₆ Xaa₁₇ Gly₁₈ Xaa₁₉ Xaa₂₀ Xaa₂₁ (SEQ ID NO: 1), or a pharmaceutically acceptable salt thereof; wherein
Xaa₁ is BE or is absent;
Xaa₂ is BK, Asn or is absent;
Xaa₃ is Asn, Ser or is absent;
Xaa₄ is Ser or is absent;
Xaa₅ is Ser, Asn, Ile, BE or is absent;
Xaa₆ is Tyr, Asp, 4-fluorophenylalanine ((4-F)Phe), BK or is absent;
Xaa₇ is Cys, cystathionine (Cth), allylglycine (Ag), 4-pentenoic acid (Pent), Hag, or Asp;
Xaa₈ is Cys, cystathionine (Cth), penicillamine (Pen), or allylglycine (Ag);
Xaa₉ is Glu, Asp, Ser, Thr, or Gln;
Xaa₁₀ is Leu, cyclohexylalanine (Cha), Phe, or 4-fluorophenylalanine ((4-F)Phe);
Xaa₁₁ is Cys, Ag, or penicillamine (Pen);
Xaa₁₂ is Cys, allylglycine (Ag), Hag, Cth, Dpr, or Val;
Xaa₁₃ is Asn or Leu;
Xaa₁₄ is Pro, Val, sarcosine (Sar), Leu, or Hydroxyproline (OH-Pro);
Xaa₁₆ is Cys, Ag, Pen or Cth;
Xaa₁₇ is Tyr, Thr, cyclohexylalanine (Cha), 4-fluorophenylalanine ((4-F)Phe), Phe, Ser, or Ala;
Xaa₁₉ is Cys, Ag or Pen;
Xaa₂₀ is Tyr, Leu, 4-fluorophenylalanine ((4-F)Phe), cyclohexylalanine (Cha), D-Tyr, N-Methyl Tyr (Nme-Tyr) or is absent;
Xaa₂₁ is absent or Asn;
wherein at least one Xaa is BE, BK, (4-F)Phe, Cth, Ag, Hag, Pen, Cha, Sar, Dpr, or OH-Pro; and
wherein the peptide contains a covalent bond between Xaa₇ and Xaa₁₂, Xaa₈ and Xaa₁₆, and Xaa₁₁ and Xaa₁₉.

53. A pharmaceutical composition for use in treating a disorder selected from colon cancer, Hereditary Nonpolyposis Colorectal Cancer (HNPCC), Lynch syndrome, gastroparesis (GP), polyps, pain, general abdominal pain, post-operative ileus, opioid-induced constipation, functional dyspepsia, diverticular disease including including but not limited to SUDD (symptomatic uncomplicated diverticular disease) and SCAD (segmental colitis associated with diverticulosis), diverticulosis, diarrhea-predominant irritable bowel syndrome, pain associated with irritable bowel syndrom (IBS), ulcerative colitis, ulcerative proctitis, Crohn's Disease, inflammatory bowel disease (IBD), chronic or acute radiation protopathy, rectal pain, chronic proctalgia, proctalgia fugax, anal pain, chronic anal fissure, post-operative anal pain, overactive bladder syndrome, stress incontinence, interstitial cystitis, bladder pain syndrome, colorectal cancer, pain associated with cancer, general pelvic pain, endometriosis, orchialgia, chronic prostatitis, prostatodynia, vulvodynia, urethral syndrome, penile pain, perianal pain and other gastrointestinal and visceral disorders, and
wherein the pharmaceutical composition comprises a peptide of any of claims 1-35.
